(19) United States
(12) Patent Application Publication
(10) Pub. No.: US 2024/0122894 A1
(43) Pub. Date: Apr. 18, 2024

(54) METHODS TO SYNTHESIZE FLAVONOID DIMERS AND OLIGOMERS AND THE USE THEREOF

(71) Applicant: National University of Singapore, Singapore (SG)

(72) Inventors: Xin Yang, Singapore (SG); Dejian Huang, Singapore (SG); Ke Du, Singapore (SG); Yi Hui Joanne Toy, Singapore (SG)

(73) Assignee: National University of Singapore, Singapore (SG)

(21) Appl. No.: 18/256,596

(22) PCT Filed: Dec. 10, 2021
(86) PCT No.: PCT/SG2021/050779

§ 371 Date: Jun. 8, 2023

(30) Foreign Application Priority Data
Dec. 10, 2020 (SG) 10202012372V

(51) Int. Cl. A61K 31/352; A61P 3/10; C07D 311/30; C07D 311/36

(52) U.S. Cl. CPC A61K 31/352; A61P 3/10; C07D 311/30; C07D 311/36

(57) ABSTRACT

Provided is a method for coupling flavonoid-containing compounds to provide flavonoid dimers, trimers and oligomers. The method comprises contacting a flavonoid-containing compound with base in the presence of air. Flavonoid dimers, trimers and oligomers may also be useful in the treatment of diseases, such as fungal infections and diseases associated with starch hydrolase.

### FIELD OF THE INVENTION

The invention relates to a method for making flavonoid dimers, trimers and oligomers, to flavonoid dimers and trimers, and their use in the treatment of diseases.

### BACKGROUND

The listing or discussion of an apparently prior-published document in this specification should not necessarily be taken as an acknowledgement that the document is part of the state of the art or is common general knowledge.

Flavonoids are a diverse class of plant secondary metabolites that have a range of bioactivity. The flavonoid core structure features a 15-carbon phenyl-chromone motif and is a privileged structure for drug discovery. The structural diversity of flavonoids stems from variable substituent positions of the phenyl, variable numbers and positions of phenol groups on the aromatic rings, number and degree of glycosylation, and formation of flavonoid dimers and oligomers.

While flavone monomers are relatively abundant in fruits and vegetables and can be extracted on an industrial scale from agri-food byproducts, flavonoid dimers and oligomers are minor components in plant biomass and are not economical to obtain in large quantities from natural sources. Synthetically, catalyzed C—C bond coupling reactions, such as Ullman reaction and Suzuki-Miyaura coupling, have been employed to make a limited number of biflavones with moderate overall yields. These high-temperature reactions suffered from major drawbacks due to their usage of toxic heavy metals, wasteful halogens and boronate by-products, and the requirement of protective groups for phenolic functional groups. The reactions also involve multiple steps which introduce additional complications and reduce yields. Therefore, these methods are not environmentally friendly and are unsuitable for large scale synthesis, or synthesis of food grade products. Furthermore, the lab synthesis of triflavonoids remains relatively unexplored.

There is therefore a need for a new method for producing flavone dimers, trimers and oligomers. Such methods may enable the production of entirely new compounds that may have useful therapeutic activity.

### SUMMARY OF THE INVENTION

The invention solves the problems in the art by providing a method for coupling flavonoid compounds under advantageously mild conditions with environmentally friendly reagents. The reactions may be performed as one-pot syntheses with high yields and regioselectivity using simple bases under mild aqueous conditions, e.g. at room temperature. These conditions are compatible with a wide range of functional groups that may be present on the flavonoids, and can be used to obtain food-grade products in an environmentally friendly way. The method of the invention may be used to make a wide range of flavonoids that were previously impossible to prepare in a lab setting.

Compounds prepared according to the invention have useful activity as antifungal agents and inhibitors of starch hydrolase.

Thus, the invention provides the following numbered clauses.
1. A method for coupling a flavonoid-containing compound, said method comprising:
   (i) providing one or more flavonoid-containing compounds; and
   (ii) contacting the one or more flavonoid-containing compounds with a base in the presence of air;
   wherein the base is selected from the group consisting of a metal carbonate, a metal hydroxide and a base of the formula R₄NOH, where each R independently represents H or C₁₋₄ alkyl; and
   step (ii) is performed in a sealed reaction vessel comprising air and a reaction mixture comprising the one or more flavonoid-containing compounds, the base and water, where the air occupies from 10 to 95% of the volume of the reaction vessel at 25° C. and a pressure of 101 kPa.
2. The method according to Clause 1, wherein the base is selected from one or more of the group consisting of lithium hydroxide, sodium hydroxide, potassium hydroxide, caesium hydroxide, sodium carbonate, potassium carbonate, and alkaline water, optionally wherein the base is selected from one or more of the group consisting of lithium hydroxide, sodium hydroxide, potassium hydroxide, caesium hydroxide, sodium carbonate, and potassium carbonate.
3. The method according to Clause 2, wherein the base is selected from one or more of the group consisting of sodium hydroxide and potassium hydroxide.
4. The method according to Clause 3, wherein the base is potassium hydroxide.
5. The method according to any one of the preceding clauses, wherein the pH of the reaction mixture in step (ii) is from about 10 to about 14, optionally a pH of from about 11 to about 13.
6. The method according to any one of the preceding clauses, wherein the temperature of the reaction mixture in step (ii) is from 15 to 30° C., optionally from 18 to 25° C.
7. The method according to any one of the preceding clauses, wherein step (ii) is performed for a time of from about 1 to about 20 hours, optionally for a time of from about 5 to about 15 hours, such as about 8 to about 12 hours.
8. The method according to any one of the preceding clauses, wherein step (ii) is performed without stirring, optionally wherein step (ii) is performed without agitating the reaction mixture.
9. The method according to any one of the preceding clauses, wherein the flavonoid-containing compound comprises a backbone selected from the group consisting of a flavone backbone, an isoflavan backbone, a neoflavonoid backbone, a flavan backbone, a flavanone backbone, an isoflavanone backbone and an isoflavone backbone, which backbone is optionally substituted with one or more (e.g. one to six, one to five, one to four, one to three, one or two, or one) substituent selected from the group consisting of hydroxyl, methoxy, glycosyl, alkoxy, NO₂, F, CN, SH, CF₃, Cl, Br, I, ═O, ═CH₂, C₁₋₁₈ alkyl, C₁₋₁₈ fluoroalkyl, —O—C(O)—R (where R represents a C₁₋₂₂ alkyl group), C₂₋₁₈ alkenyl, prenyl, phytyl, exocyclic C₃₋₆ cycloalkyl, exocyclic C₅₋₆ cycloalkenyl, phenyl, phenoxyl, C₁₋₁₈ N-aklylaminyl, and C₁₋₁₈ N,N-dialkylaminyl,

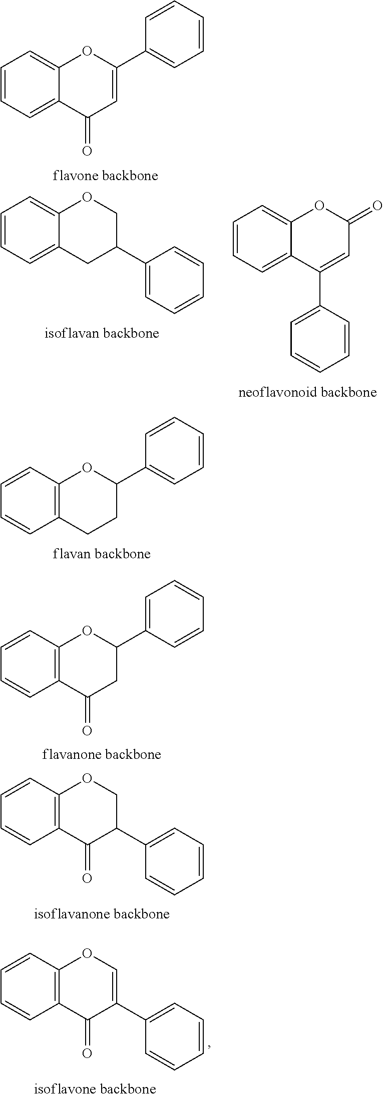

or wherein the flavonoid-containing compound comprises a dimer or trimer thereof.
10. The method according to any one of the preceding clauses, wherein the flavonoid-containing compound comprises a flavone backbone.
11. The method according to any one of the preceding clauses, wherein the flavonoid-containing compound is a flavonoid monomer.
12. The method according to any one of the preceding clauses, wherein the flavonoid-containing compound is a flavonoid dimer.
13. The method according to any one of the preceding clauses, wherein the flavonoid-containing compound is a flavonoid trimer, optionally wherein the coupling is an intramolecular coupling.
14. The method according to any one of the preceding clauses, wherein the air occupies from 30 to 90% (e.g. from 40 to 80%) of the volume of the reaction vessel at 25° C. and a pressure of 101 kPa.
15. A method according to any one of Clauses 1 to 14, wherein the flavonoid is selected from the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, 3′,4′-dihydroxyflavone, a dimer formed from two of the foregoing, and a trimer formed from three of the foregoing.
16. A compound selected from the group consisting of

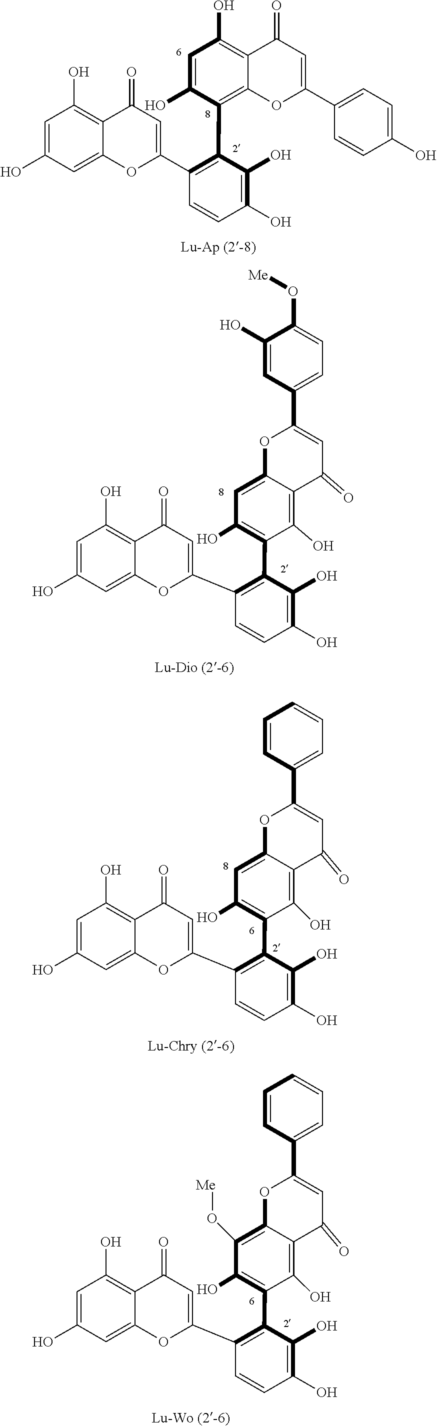

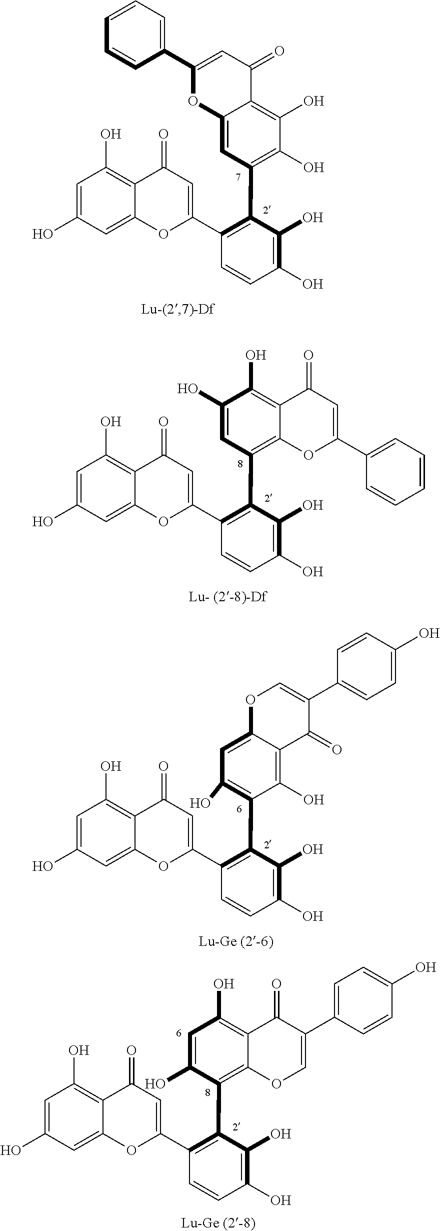

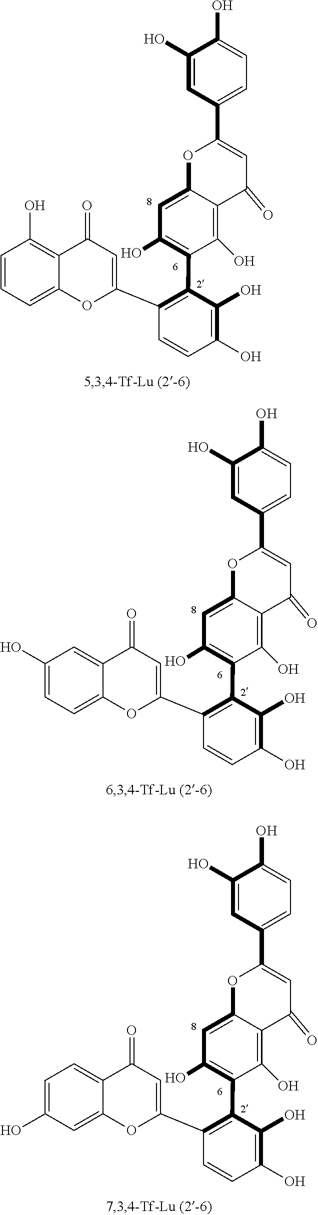

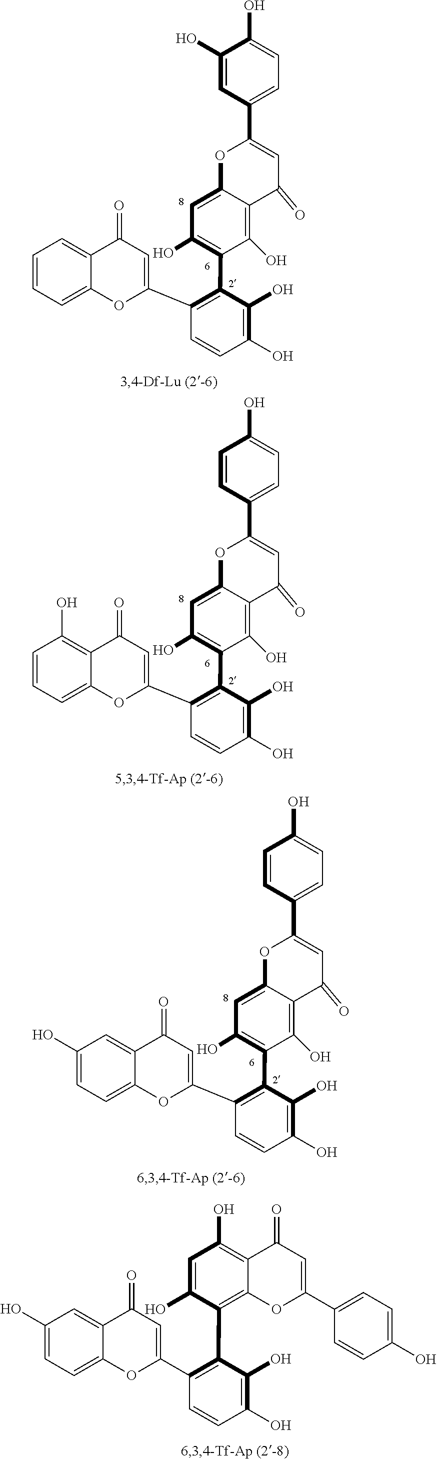

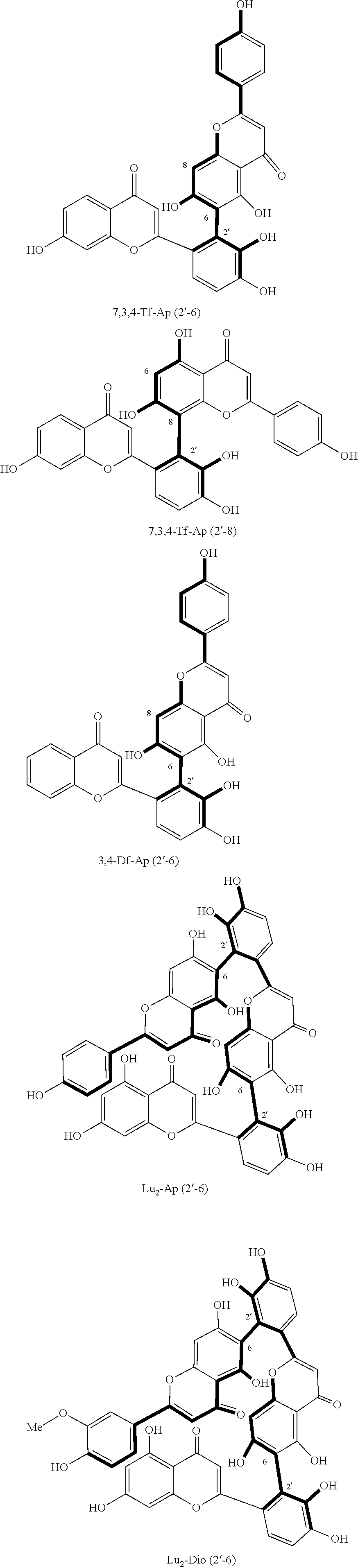

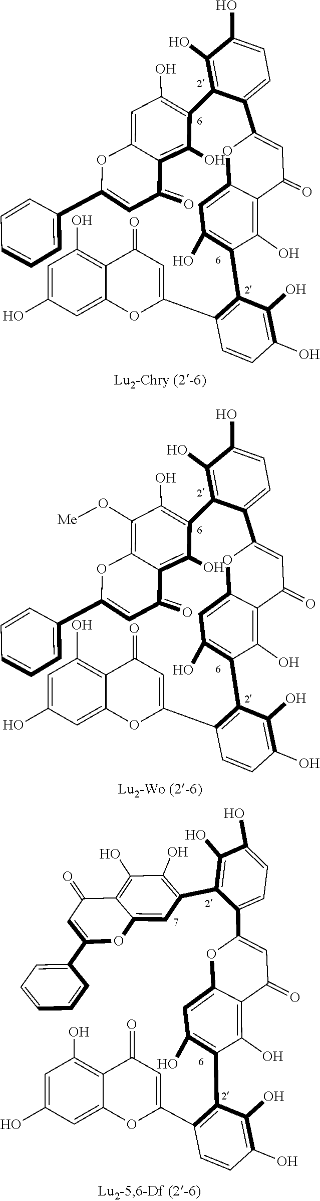

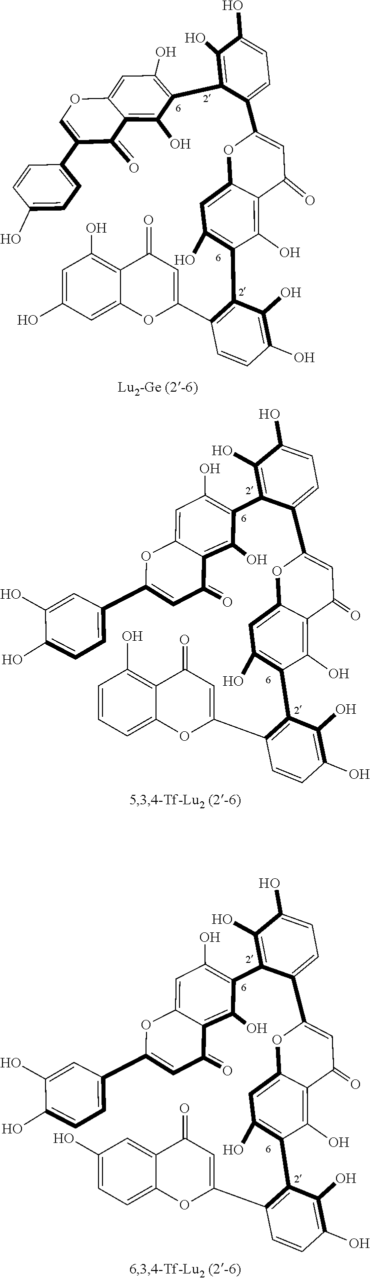

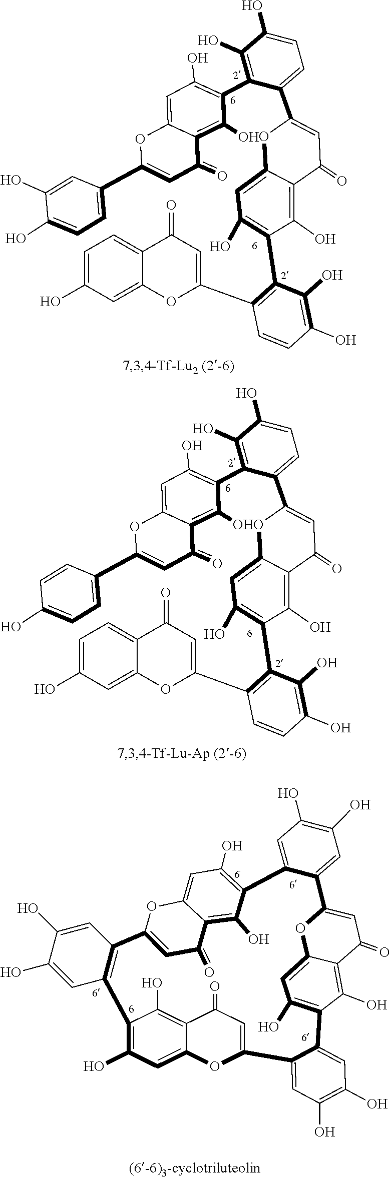

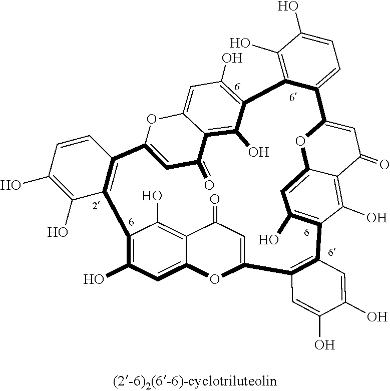

and pharmaceutically acceptable salts thereof.
17. Use of a compound that is:
   (a) a dimer or trimer formed from one, two or three flavonoids selected from of the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, and 3′,4′-dihydroxyflavone, or
   (b) a pharmaceutically acceptable salt thereof,
in the manufacture of a medicament for the treatment of:
   (i) a fungal infection (e.g. a candida infection); or
   (ii) disorder or condition ameliorated by the inhibition of starch hydrolase (e.g. hyperglycaemia, diabetes and obesity).
18. Use according to Clause 17, wherein the compound is a compound or pharmaceutically acceptable salt according to Clause 16.
19. Use according to Clause 17, wherein the compound is a compound selected from the group consisting of:

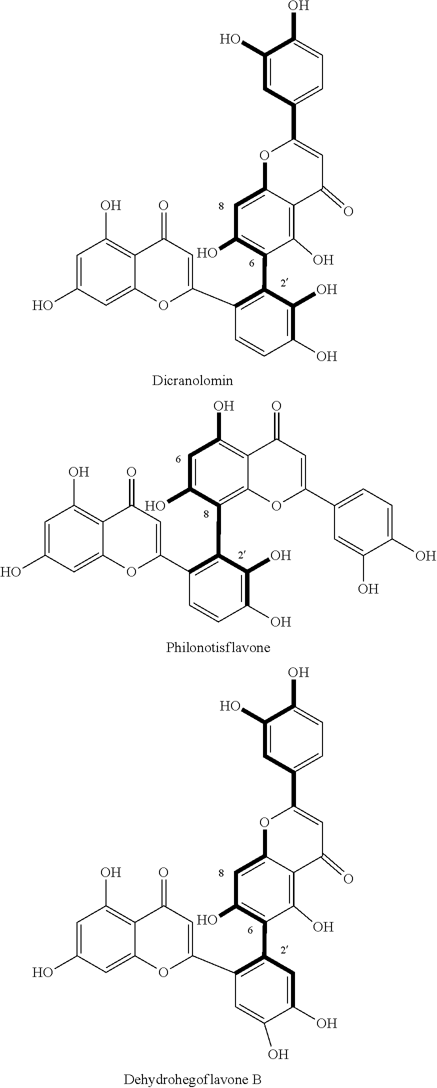

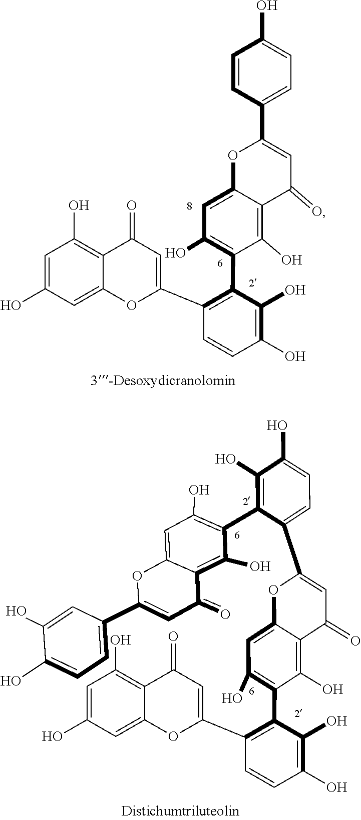

and pharmaceutically acceptable salts thereof.
20. Use according to Clause 19, wherein the compound is selected from dicranolamin, 3′″-Desoxydicranolomin, distichumtriluteolin, and pharmaceutically acceptable salts thereof,
   optionally wherein the compound is selected from dicranolamin, 3′″-Desoxydicranolomin, and pharmaceutically acceptable salts thereof
   more optionally wherein the compound is dicranolamin or a pharmaceutically acceptable salt thereof.

### BRIEF DESCRIPTION OF THE FIGURES

FIG. 1 depicts the synthesis of nature-occurring and unnatural biflavones and triflavones. (A) Suzuki-Miyaura coupling reaction in the synthesis of bioflavonoids; (B) Ullmann coupling reaction in the synthesis of bioflavonoids; and (C) This work: oxygen mediated oxidative coupling reaction in the synthesis of flavonoids oligomers; examples include 31 biflavones and 11 triflavones.

FIG. 2 depicts the oxygen mediated oxidative coupling of flavones. (A) Luteolin undergoes oxidative coupling reaction, under weakly alkaline water to major product dicranolomin (2a) and minor products, philonotisflavone (2a′, structure not shown), dehydrohegoflavone B (2a″) and distichumtriluteolin (3a), which could be obtained separately from the coupling of isolated 2a and luteolin with 42% isolated yield; (B) Intramolecular oxidative coupling 3a proceeds to give different isomers cyclotriluteolin (4a, 4a′, and 4a″) and (C) The solid-state structure of one isomer 4a′ was determined by single-crystal X-ray diffraction.

FIG. 3 depicts (A) ring rearrangement of cyclotriluteolin (CTL); (B) variable temperature NMR of 4a; (C) proposed intramolecular ring rearrangement of cyclotriluteolin; and (D) calculated Gibbs free energies of cyclotriluteolin isomers. Calculations were performed at the M06-2X/6-311+G(d,p), SMD(H₂O)//M06-2X/6-31G(d)SMD(H₂O) level of theory. Energies are in kcal·mol⁻¹.

FIG. 4 depicts the characterization of 7,3′,4′-trihydroxyflavone radical anion. (A) The formation of 7,3′,4′-trihydroxyflavone radical anion; (B) Experimental and fitted EPR spectrum of FH″ recorded at 295 K; (C) Experimental hyperfine coupling constants for F^{-·}; (D) Spin-density distribution in FH^{-·} predicted with density functional theory (DFT, UM062x/6-311+G(d,p)).

FIG. 5 depicts the electron spin resonance (ESR) spectrum of luteolin in aqueous potassium hydroxide (KOH) solutions under (A) pH 11.5; and (B) pH 12.5. (i) The electron paramagnetic resonance (EPR) spectrum of luteolin dissolved in H₂O¹⁶ and exposed in oxygen O₂¹⁶; (ii) The EPR spectrum of luteolin dissolved in H₂O¹⁶ and exposed in oxygen O₂¹⁷; and (iii) The EPR spectrum of luteolin dissolved in H₂O¹⁷ and exposed in oxygen O₂¹⁶.

FIG. 6 depicts the ESR spectrum of luteolin in aqueous KOH solutions under pH 12.5. (A) Experimental and simulated EPR spectrum of luteolin dissolved in H₂O¹⁷; (B) Spin-density distribution in LH₂O^{-·} predicted with DFT (UM062x/6-311+G(d,p)); and (C) Proposed mechanism of the formation of LH₂O^{-·} radical anion.

FIG. 7 depicts the characterization of dicranolomin radical anion. (A) The formation of 3′, 4′ dihydroxyflavone radical anion; (B) Experimental and fitted EPR spectrum of F^{-·} recorded at 295 K; (C) Proposed structure for dicranolomin radical anion DH₈^{·-}; and (D) Spin-density distribution in DH₈^{-·} predicted with DFT (UM062x/6-311+G(d,p)).

FIG. 8 depicts the yields of dimers from homo cross-coupling reaction of B-catechol flavones: 5,3′,4′-trihydroxyflavone, 6,3′,4′-trihydroxyflavone, 7,3′,4′-trihydroxyflavone, 7,8,3′,4′-tetrahydroxyflavone and luteolin.

FIG. 9 depicts the nucleophilicity of B-catechol flavones. The global nucleophilicity index (N_{Nu}) was calculated by the DFT method at the B3LYP/6-311++G(d,p) level of theory. The energy of the highest-occupied molecular orbital (HOMO) was referenced against the HOMO energy of tetracyanoethylene (TCE) according N_{Nu}=E_{HOMO(Nu)}−E_{HOMO(TCE)} (L. R. Domingo et al., J. Org. Chem. 2008, 73, 4615-4624).

FIG. 10 depicts the substrate scope of oxygen mediated cross-coupling of luteolin and flavones. (A) Luteolin as a radical precursor and acceptor; and (B) B-catechol flavones as a radical precursor. All yields were isolated and selectivity determined by HPLC analysis. brsm=yields based on the recovery of starting materials. R═H if not specified.

FIG. 11 depicts the substrate scope of oxidative coupling for the synthesis of triflavones in alkaline water. (A) Dicranolomin as a radical precursor and acceptor; and (B) B-catechol biflavone as a radical precursor. All yields are isolated yields. brsm=yields based on the recovery of starting materials. R═H if not specified.

FIG. 12 depicts (A) the sample preparation of luteolin for pKₐ calculation; and (B) experimental set for sample preparation.

FIG. 13 depicts the (A) illustration; and (B) picture of the oxygen consumption monitoring set. The pressure changes of reaction under stirring and no stirring was recorded by pressure gauge (NVISION, pressure recorder with vacuum range of 30 MPa, CRYSTAL engineering corporation).

FIG. 14 depicts the effects of pH on the yields of luteolin cross-coupling reaction with (A) luteolin; (B) apigenin (Ap); (C) diosmetin (Dio); (D) chrysin (Chry); (E) wogonin (Wo); (F) 5,6-dihydroxyflavone; (G) genistein (Ge); (H) 5,3′,4′-trihydroxyflavone; (1) 6,3′,4′-trihydroxyflavone; and (J) 7,3′,4′-trihydroxyflavone.

FIG. 15 depicts the effects of pH on the yields of dicranolomin cross-coupling reaction with (A) luteolin; (B) Ap; (C) Dio; (D) Chry; (E) Wo; (F) 5,6-dihydroxyflavone; (G) Ge; (H) 5,3′,4′-trihydroxyflavone; (1) 6,3′,4′-trihydroxyflavone; and (J) 7,3′,4′-trihydroxyflavone.

FIG. 16 depicts pKₐ measurement of luteolin using nuclear magnetic resonance (NMR). pKₐ₁, PKₐ₂, PKₐ₃ and pKₐ₄ determination of luteolin based on dependence of ¹³C NMR chemical shifts of (A) C4′; (B) C7; (C) C3′; and (D) C5 of luteolin on pH. The lines represent computer fits to the Henderson-Hasselbalch equation with the pKₐ values; and (E) The distribution curve of luteolin species in aqueous solution was generated based on pKₐ values FIG. 17 depicts the pKₐ profile of luteolin and the radicals generated. (A) The distribution curve of luteolin species in aqueous solution LuH₄: luteolin, LuH₃⁻: monoanion, LuH₂²⁻: dianion, LuH³⁻: trianion, and Lu⁴⁻: tetraanion; (B) Experimental and simulated EPR spectrum of LuH^{·2-}; (C) Spin-density distribution in LuH^{·2-} predicted with DFT (UM062X/6-311+G(d,p)); (D) Experimental EPR spectrum of (i) LuOH^{·2-} in air-saturated H₂O; (ii) in H₂O with 30% ¹⁷O₂-enriched molecular oxygen; and (iii) in 30% ¹⁷O enriched water; and (E) Proposed mechanism for the formation of LuOH^{·2-}.

FIG. 18 depicts the pH dependent ESR spectrum of luteolin (15.0 mM) in aqueous KOH solutions. The determination was conducted immediately when luteolin was mixed with KOH solution at specific concentrations, and the pH was detected after EPR test.

FIG. 19 depicts the reaction mechanism and computational study of regio-selectivity. (A) Proposed mechanisms for oxidative coupling reactions, counter-cations are omitted for clarity; (B) Computed Gibbs energy difference between 2a and 2a′; and (C) The impact of counter-cations on the yields coupling reaction of luteolin.

FIG. 20 depicts the characterization of 3′,4′-dihydroxyflavone radical anion. (A) The formation of 3′,4′dihydroxyflavone radical anion; (B) Experimental and fitted EPR spectrum of F^{-·} recorded at 295 K; (C) Experimental hyperfine coupling constants for F^{-·}; and (D) Spin-density distribution in F^{-·} predicted with DFT (UM062x/6-311+G(d,p)).

FIG. 21 depicts the characterization of 5,3′,4′-trihydroxyflavone radical anion. (A) The formation of 5,3′,4′-trihydroxyflavone radical anion; (B) Experimental and fitted EPR spectrum of FH^{-·} recorded at 295 K; (C) Experimental hyperfine coupling constants for F^{-·}; and (D) Spin-density distribution in FH^{-·} predicted with DFT (UM062x/6-311+G(d,p)).

FIG. 22 depicts the characterization of 6,3′,4′-trihydroxyflavone radical anion. (A) The formation of 6,3′,4′-trihydroxyflavone radical anion; (B) Experimental and fitted EPR spectrum of FH^{-·} recorded at 295 K; (C) Experimental hyperfine coupling constants for F^{-·}; and (D) Spin-density distribution in FH^{-·} predicted with DFT (UM062x/6-311+G(d,p)).

FIG. 23 depicts the concentration changes over reaction time for (A) luteolin (1a); (B) 2a; and (C) distichumtriluteolin (3a) in luteolin-luteolin cross-coupling reaction with stirring and without stirring.

FIG. 24 depicts the oxygen consumption curve of luteolin-luteolin cross-coupling reaction under stirring and no stirring. Luteolin (57.2 mg, 0.1 mmol) was added to a 250 mL round bottle flask. Then, KOH solution (50.0 mL, 0.03 M) was introduced to start the reaction. The reaction was conducted in real-time monitoring in an air-tight system.

FIG. 25 depicts the bioactivity of luteolin and flavone dimers and trimers. (A) Image of surviving Aspergillus niger colony in the present of luteolin (1a), 2a, distichumtriluteolin (3a) and amphotericin B (AmB), and degrees of inhibition (%); (C) The docking of 2a in complex with α-amylase [Protein Data Bank (PDB) 3GBN]. 2a is shown in a ball-and-stick representation, with whole amylase protein rendered in surface representation. A magnified view of 2a binding site in the enzyme is shown. Overlay of the structure of 2a in complex with interacting loop residues; (D) Catalytic mechanism of α-amylase. Reaction occurs with acid/base and nucleophilic assistance provided by amino acid side chains from Asp197, Glu233 and Asp300; and (E) and (F) show the polar interactions in α-amylase, with the interactions of FI and FII part of 2a [labeled in (D)] measured in A.

FIG. 26 depicts the starch hydrolysis inhibition activities of flavonoid oligomers showed in acarbose equivalent. (A) The α-amylase inhibition activity of flavonoids oligomers; and (B) The α-glucosidase inhibition activity of flavonoids oligomers.

FIGS. 27a and 27b depicts the representative kinetic curves and dose response curve of α-amylase and α-glucosidase in the presence of luteolin (1a), its derivatives, biflavone (2a) and triflavone (3a). (a, b, e, f, i and j) show the starch hydrolysis activity of flavonoids with α-amylase while (c, d, g, h, k and i) show the starch hydrolysis activity of flavonoids with α-glucosidase.

FIGS. 28a and 28b depict the representative kinetic curves and dose response curve of α-amylase and α-glucosidase in the presence of luteolin (1b), its derivatives, biflavone (2b) and triflavone (3b). (a, b, e, f, i and j) show the starch hydrolysis activity of flavonoids with α-amylase while (c, d, g, h, k and i) show the starch hydrolysis activity of flavonoids with α-glucosidase.

FIGS. 29a and 29b depict the representative kinetic curves and dose response curve of α-amylase and α-glucosidase in the presence of luteolin (1c), its derivatives, biflavone (2c) and triflavone (3c). (a, b, e, f, i and j) show the starch hydrolysis activity of flavonoids with α-amylase while (c, d, g, h, k and i) show the starch hydrolysis activity of flavonoids with α-glucosidase.

FIGS. 30a and 30b depict the representative kinetic curves and dose response curve of α-amylase and α-glucosidase in the presence of luteolin (1d), its derivatives, biflavone (2d) and triflavone (3d). (a, b, e, f, i and j) show the starch hydrolysis activity of flavonoids with α-amylase while (c, d, g, h, k and i) show the starch hydrolysis activity of flavonoids with α-glucosidase.

FIGS. 31a and 31b depict the representative kinetic curves and dose response curve of α-amylase and α-glucosidase in the presence of luteolin (1e), its derivatives, biflavone (2e) and triflavone (3e). (a, b, e, f, i and j) show the starch hydrolysis activity of flavonoids with α-amylase while (c, d, g, h, k and i) show the starch hydrolysis activity of flavonoids with α-glucosidase.

FIGS. 32a and 32b depict the representative kinetic curves and dose response curve of α-amylase and α-glucosidase in the presence of luteolin (1f), its derivatives, biflavone (2f) and triflavone (3f). (a, b, e, f, i and j) show the starch hydrolysis activity of flavonoids with α-amylase while (c, d, g, h, k and i) show the starch hydrolysis activity of flavonoids with α-glucosidase.

FIGS. 33a and 33b depict the representative kinetic curves and dose response curve of α-amylase and α-glucosidase in the presence of luteolin (1g), its derivatives, biflavone (2g) and triflavone (3g). (a, b, e, f, i and j) show the starch hydrolysis activity of flavonoids with α-amylase while (c, d, g, h, k and i) show the starch hydrolysis activity of flavonoids with α-glucosidase.

FIGS. 34a and 34b depict the representative kinetic curves and dose response curve of α-amylase and α-glucosidase in the presence of luteolin (1h), its derivatives, biflavone (2h) and triflavone (3h). (a, b, e, f, i and j) show the starch hydrolysis activity of flavonoids with α-amylase while (c, d, g, h, k and i) show the starch hydrolysis activity of flavonoids with α-glucosidase.

FIGS. 35a and 35b depict the representative kinetic curves and dose response curve of α-amylase and α-glucosidase in the presence of luteolin (ii), its derivatives, biflavone (2i) and triflavone (3i). (a, b, e, f, i and j) show the starch hydrolysis activity of flavonoids with α-amylase while (c, d, g, h, k and i) show the starch hydrolysis activity of flavonoids with α-glucosidase.

FIGS. 36a and 36b depicts the representative kinetic curves and dose response curve of α-amylase and α-glucosidase in the presence of luteolin (1j) its derivatives, biflavone (2j) and triflavone (3j). (a, b, e, f, i and j) show the starch hydrolysis activity of flavonoids with α-amylase while (c, d, g, h, k and i) show the starch hydrolysis activity of flavonoids with α-glucosidase.

FIG. 37 depicts the starch hydrolase inhibition activity of biflavones synthesized compared with luteolin monomer. AE=acarbose equivalence (based on molar concentration). α-Amylase solution (2 U/mL in buffer, 20 μL) and α-glucosidase solution (1×10⁻² UmL in buffer) were applied in this assay and the concentration of starch for this assay is 20 mg/mL. Lu₂=biluteolin (2-6 linkage), Lu₂′=biluteolin (2′-8 linkage), Lu-Ap, luterolin-apigenin dimer (2′-6 linkage), Lu-Dio, luteolin diosmetin dimer (2-6 linkage), Lu-Chry, luteolin-chrysin dimer (2′-6 linkage).

FIG. 38 depicts the visualization of the docking result between amylase and ligands: acarbose, 2a, 3′″-desoxydicranolomin (2b), 1a and 3a.

FIG. 39 depicts the visualization of the docking result between amylase and ligands: 2b. (A) The crystal structure of 2b in complex with α-amylase [Protein Data Bank (PDB) 1ppi]. (B) 2b is shown in a ball-and-stick representation; (C) A magnified view of acarbose binding sites in the enzyme is shown; and (D-E) The polar interactions in the 2b-1ppi complex, with the interactions of 2b measured in A.

FIG. 40 depicts the Visualization of the docking result between amylase and ligands: acarbose. (A) The crystal structure of 2a in complex with α-amylase [Protein Data Bank (PDB) 1ppi]; (B) Acarbose is shown in a ball-and-stick representation; (C) A magnified view of acarbose binding sites in the enzyme is shown; and (D-E) The polar interactions in the acarbose-1ppi complex, with the interactions of acarbose measured in A.

FIG. 41 depicts (A) Blood glucose concentration of mice with treatment of YX2 (luteolin dimer, 2a); and (B) The blood glucose increasement of mice with treatment of YX2 (luteolin dimer 2a).

### DETAILED DESCRIPTION

The word “comprising” refers herein may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “an oxygen carrier” includes mixtures of two or more such oxygen carriers, reference to “the catalyst” includes mixtures of two or more such catalysts, and the like.

The invention provides a method for coupling a flavonoid-containing compound, said method comprising:
(i) providing one or more flavonoid-containing compounds; and
(ii) contacting the one or more flavonoid-containing compounds with a base in the presence of air;
   wherein the base is selected from the group consisting of a metal hydroxide and a base of the formula R₄NOH, where each R independently represents H or C₁₋₄ alkyl; and step (ii) is performed in a sealed reaction vessel comprising air and a reaction mixture comprising the one or more flavonoid-containing compounds, the base and water, where the air occupies from 10 to 95% of the volume of the reaction vessel at 25° C. and a pressure of 101 kPa.

As used herein, a “flavonoid-containing compound” is a compound that contains a flavonoid carbon skeleton, which compound may be substituted with additional carbon-containing functional groups. A flavonoid carbon skeleton comprises a benzene ring fused to a 6-membered heterocyclic ring containing an oxygen atom, which heterocyclic ring is bonded to a phenyl ring by a C—C bond:

The carbon atoms of the 6-membered heterocyclic ring containing an oxygen atom that do not form part of the fused benzene ring may be connected by single or double bonds as shown in the structure above (provided that any one carbon atom in the heterocyclic ring forms part of only one double bond). The oxygen atom may be bonded to the non-aromatic carbon atom by a single or double bond. When this is a double bond, the oxygen atom will have a positive charge, and the compound will comprise a balancing negative charge or a counterion.

Flavonoids may contain a wide range of functional groups bonded to the above skeleton, for example one or more (e.g. one to twelve, one to eleven, one to ten, one to nine, one to eight, one to seven, one to six, one to five, one to four, one to three, one or two, or one) substituents selected from the group consisting of hydroxyl, methoxy, glycosyl, alkoxy, NO₂, F, CN, SH, CF₃, Cl, Br, I, ═O, ═CH₂, C₁₋₁₈ alkyl, C₁₋₁₈ fluoroalkyl, C₂₋₁₈ alkenyl, —O—C(O)—R (where R represents a C₁₋₂₂ alkyl group), prenyl, phytyl, exocyclic C₃₋₆ cycloalkyl, exocyclic C₅₋₆ cycloalkenyl, phenyl, phenoxyl, C₁₋₁₈ N-aklylaminyl, and C₁₋₁₈ N,N-dialkylaminyl.

Specific examples of flavonoid backbones that may be present in flavonoid-containing compounds include the following:

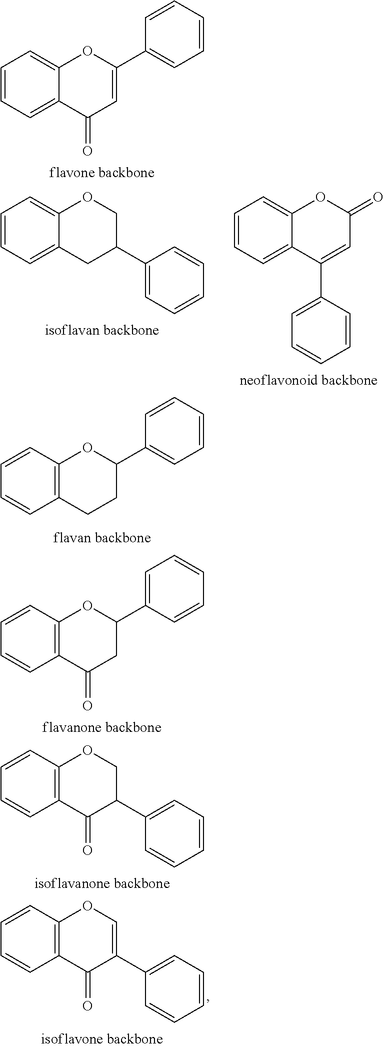

and dimers and trimers thereof. As will be appreciated by a person skilled in the art, in this context a dimer or trimer thereof includes dimers/trimers formed from two/three of the same backbone, or formed from two/three different backbones.

In specific embodiments of the invention, the flavonoid may be selected from the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, 3′,4′-dihydroxyflavone, a dimer formed from two of the foregoing, and a trimer formed from three of the foregoing. In other words, the flavonoid-containing compound may represent an optionally substituted flavonoid selected from the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, 3′,4′-dihydroxyflavone, a dimer formed from two of the foregoing, and a trimer formed from three of the foregoing.

In specific embodiments of the invention, the flavonoid-containing compound may comprise a flavone backbone. In some embodiments of the invention the flavonoid-containing compound may be a monomer. In some embodiments of the invention the flavonoid-containing compound may be a dimer. In some embodiments of the invention the flavonoid-containing compound may be a trimer.

When the flavonoid-containing compound is a trimer, the coupling reaction of the invention may be an intramolecular reaction to provide a cyclic trimer. Alternatively (or in addition), the coupling reaction of the invention may couple a trimer with another flavonoid-containing compound, such as a monomer, to produce a tetramer.

The method involves contacting the flavonoid-containing compound with a base selected from the group consisting of a metal carbonate, a metal hydroxide and a base of the formula R₄NOH, where each R independently represents H or C₁₋₄ alkyl. Examples of bases that may be useful in embodiments of the invention include one or more of the group consisting of lithium hydroxide, sodium hydroxide, potassium hydroxide, caesium hydroxide, sodium carbonate, potassium carbonate, and alkaline water. As set out in the below Examples, the nature of the counterion of the base can have an effect on the yield provided by the reaction. Therefore, in some embodiments it may be preferable for the base to be selected from the group consisting of alkali metal hydroxides. In further embodiments, the base may be selected from the group consisting of lithium hydroxide, sodium hydroxide, potassium hydroxide and caesium hydroxide. In yet further embodiments, the base may be selected from the group consisting of sodium hydroxide and potassium hydroxide. In a specific embodiment, the base may be potassium hydroxide.

The base results in an alkaline reaction mixture. The reaction mixture may have a pH of from about 10 to about 14, such as from about 11 to about 13.

For the avoidance of doubt, it is explicitly contemplated that the end point of any range herein may be combined with any end point of another range for the same variable. Thus, for the above pH ranges, the invention explicitly contemplates the following pH ranges: from about 10 to about 11; from about 10 to about 13; from about 10 to about 14; from about 11 to about 13; from about 11 to about 14; and from about 13 to about 14.

Step (ii) of the method is performed in a sealed reaction vessel. This enables the amount of oxygen to which the reaction mixture is exposed to be controlled. If insufficient oxygen is present then the coupling reaction will not run to completion, and provide unsatisfactory yields. However, if too much oxygen is present then over-oxidation may occur. For this reason, the invention involves the use of a sealed reaction vessel, where from 10 to 95% of the volume of the reaction vessel is occupied by air (when assessed at 25° C. and a pressure of 101 kPa). In some embodiments of the invention, air may occupy from 30 to 90% (e.g. from 40 to 80%) of the volume of the reaction vessel at 25° C. and a pressure of 101 kPa. As will be appreciated by a person skilled in the art this temperature and pressure refer to the conditions for assessing the volume occupied by air in the reaction vessel, and do not limit the temperature or pressure at which the reaction may take place.

For similar reasons, the method of the invention may in some embodiments be performed without stirring (or without agitating the reaction mixture). Stirring the reaction mixture increases contact between the reaction mixture and air in the reaction vessel, increasing the rate at which additional oxygen is dissolved into the reaction mixture—and possibly leading to over-oxidation. The inventors surprisingly found that improved yields may be obtained when the method is performed in a sealed reaction vessel without stirring.

The reaction mixture comprises water. Water may advantageously be used as a solvent because it is environmentally friendly, cheap and safe. All of the reagents used in the method are stable in water, and the use of water allows the production of food-grade products. Nevertheless, a skilled person will understand that the method would be possible with other solvents.

Step (ii) of the method may be performed under a range of temperatures, for example from 0° C. to 50° C. (provided the reaction mixture does not freeze at 0° C.). In specific embodiments of the invention step (ii) of the method may be performed at a temperature of from 15 to 30° C., such as from 18 to 25° C. Step (ii) may also be performed for a varying length of time, for example from about 1 to about 20 hours, from about 5 to about 15 hours, such as about 8 to about 12 hours.

The invention provides certain flavonoid compounds selected from the group consisting of

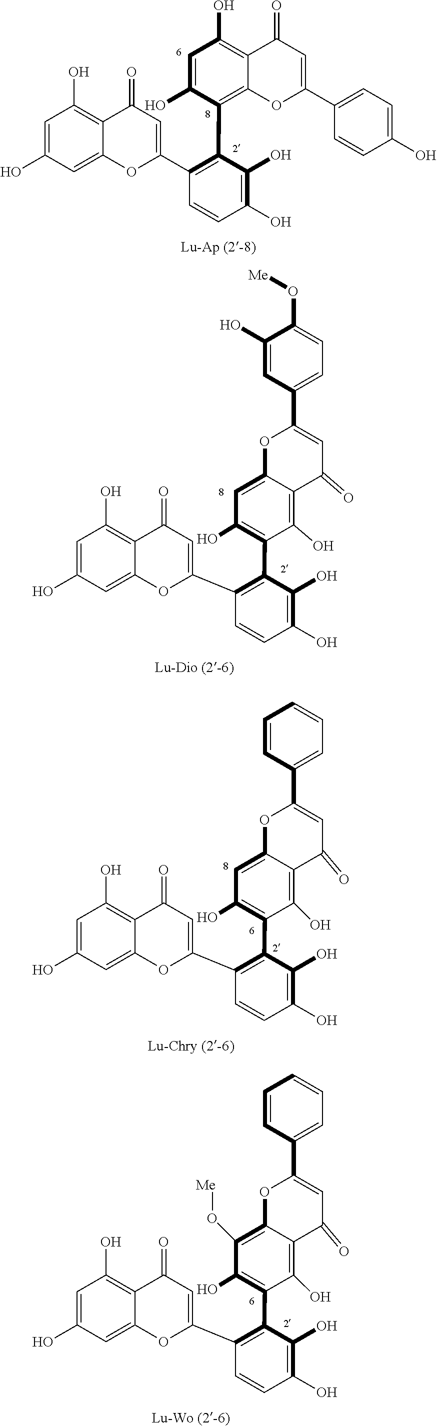

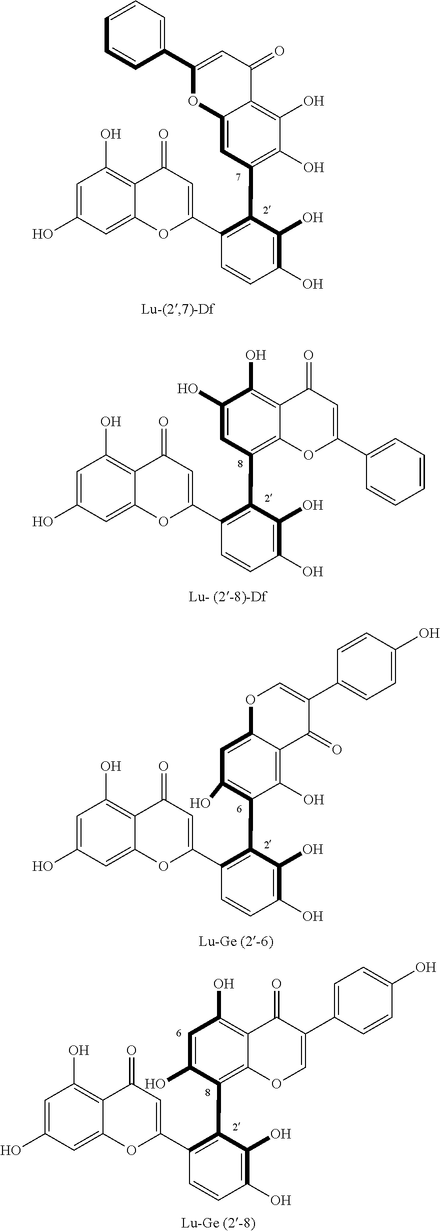

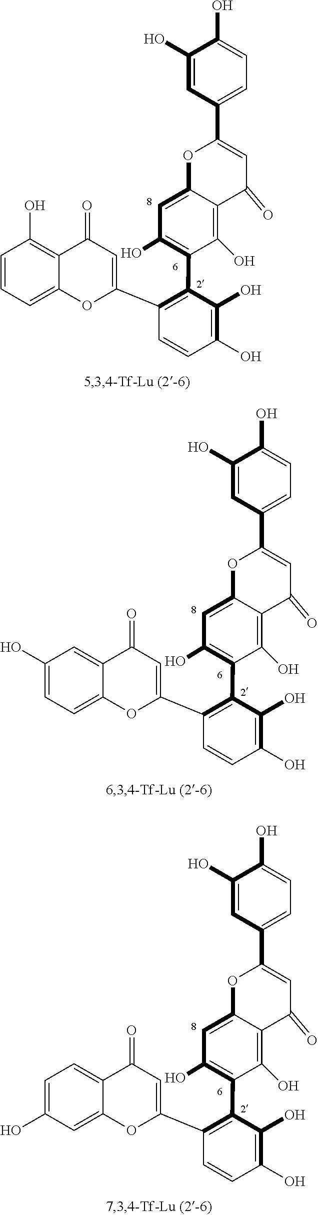

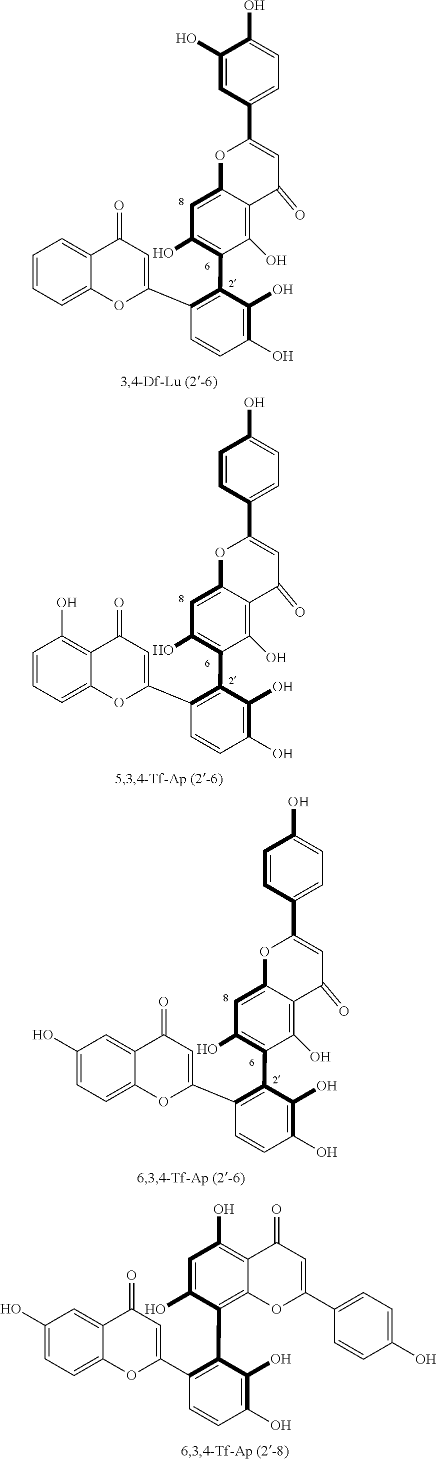

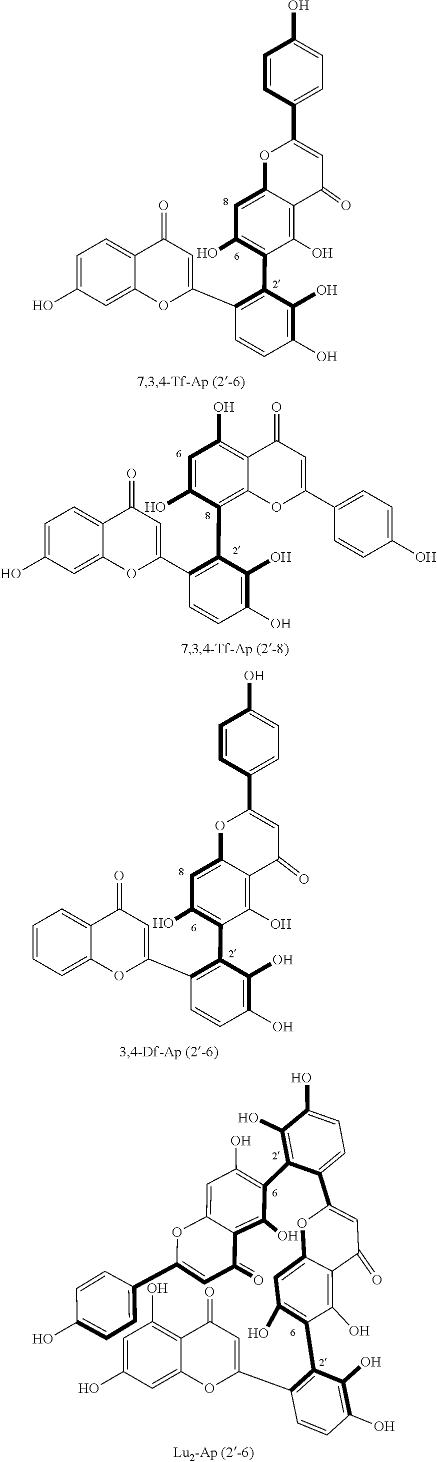

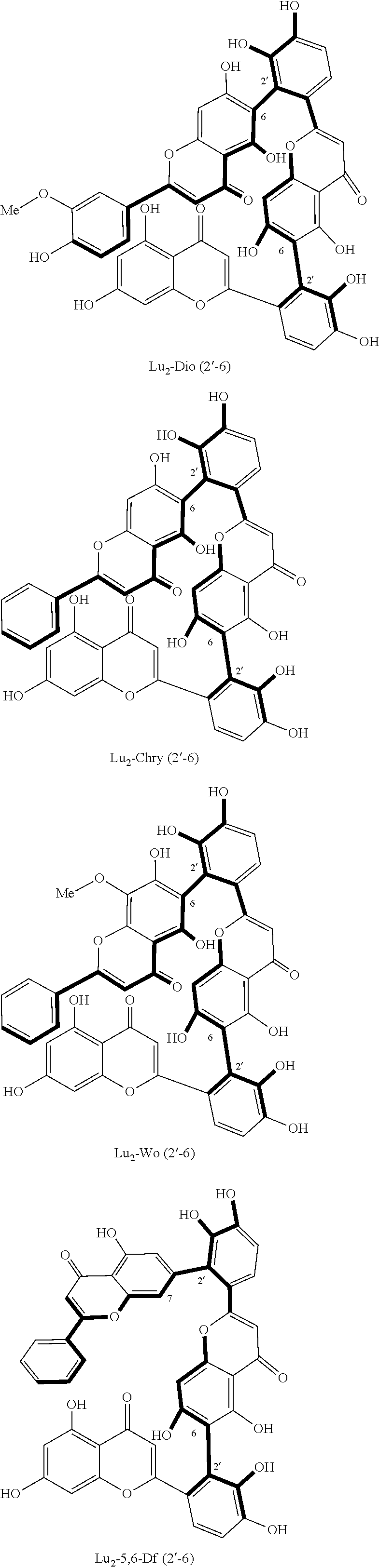

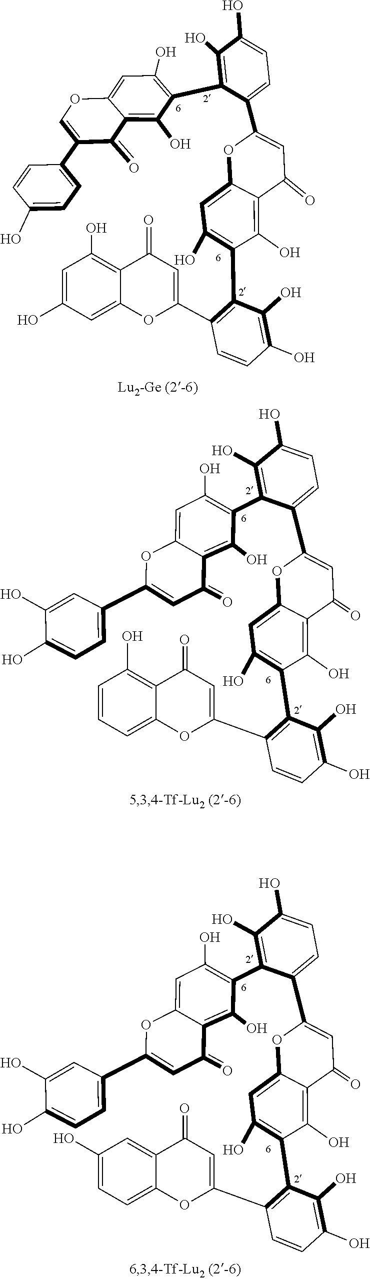

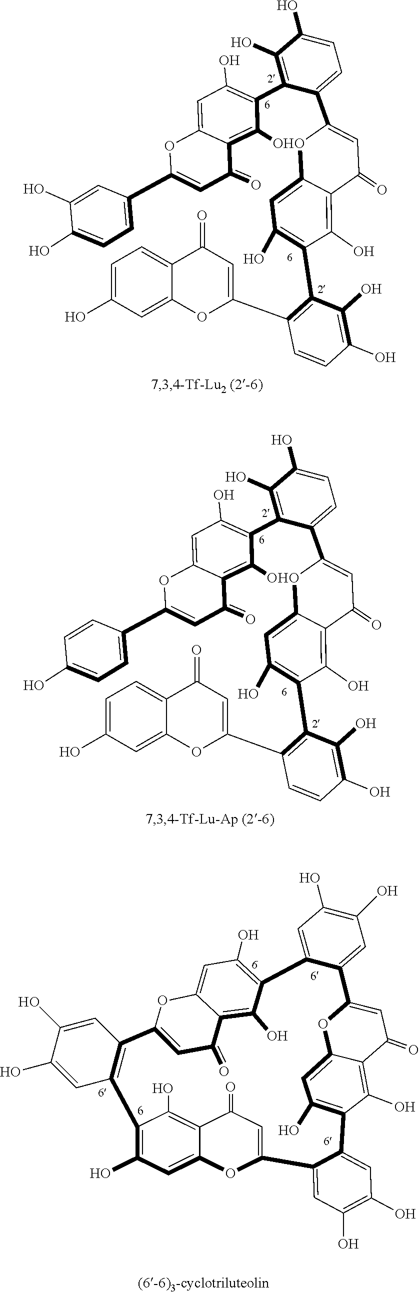

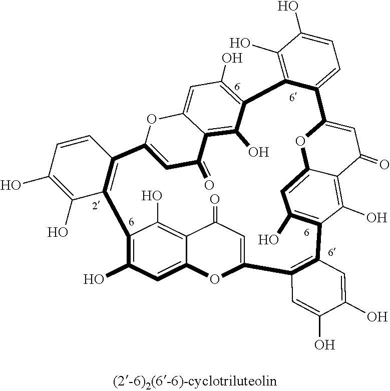

and pharmaceutically acceptable salts thereof.

Such compounds may be prepared from their constituent monomers/dimers using the method of the invention. In the case of the cyclic trimers, they may be made from the acyclic trimers, which may themselves be made from their constituent monomers/dimers using the method of the invention.

The dimers and trimers made according to the invention may be useful in methods of medical treatment. Thus, the invention also provides:
(1) the use of a compound that is:
   (a) a dimer or trimer formed from one, two or three flavonoids selected from of the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, and 3′,4′-dihydroxyflavone, or
   (b) a pharmaceutically acceptable salt thereof,
   in the manufacture of a medicament for the treatment of:
   (i) a fungal infection (e.g. a candida infection); or
   (ii) disorder or condition ameliorated by the inhibition of starch hydrolase (e.g. hyperglycaemia, diabetes and obesity).
(2) a compound that is:
   (a) a dimer or trimer formed from one, two or three flavonoids selected from of the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, and 3′,4′-dihydroxyflavone, or
   (b) a pharmaceutically acceptable salt thereof,
   for use in the treatment of:
   (i) a fungal infection (e.g. a candida infection); or
   (ii) disorder or condition ameliorated by the inhibition of starch hydrolase (e.g. hyperglycaemia, diabetes and obesity).
(3) a method of treatment of a disorder or condition selected from:
   (i) a fungal infection (e.g. a candida infection); or
   (ii) disorder or condition ameliorated by the inhibition of starch hydrolase (e.g. hyperglycaemia, diabetes and obesity),
   which method comprises the administration of an effective amount of a compound that is:
   (a) a dimer or trimer formed from one, two or three flavonoids selected from of the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, and 3′,4′-dihydroxyflavone, or
   (b) a pharmaceutically acceptable salt thereof,
      to a patient in need of such treatment.

In the above medical uses (1), (2) and (3) according to the invention, the compound may be a compound according to the invention. For example, the compound may be a compound selected from the group consisting of

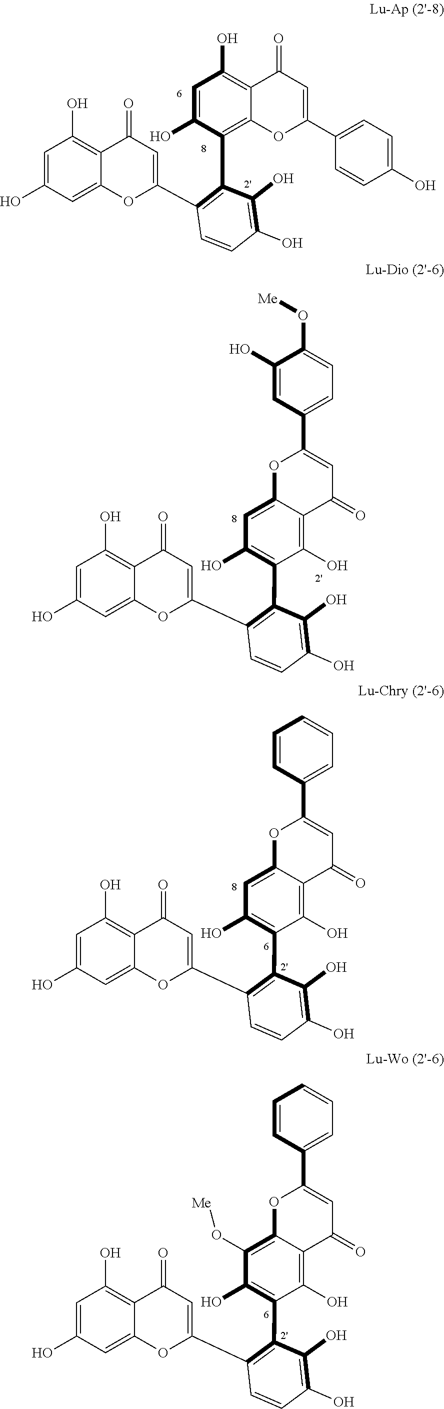

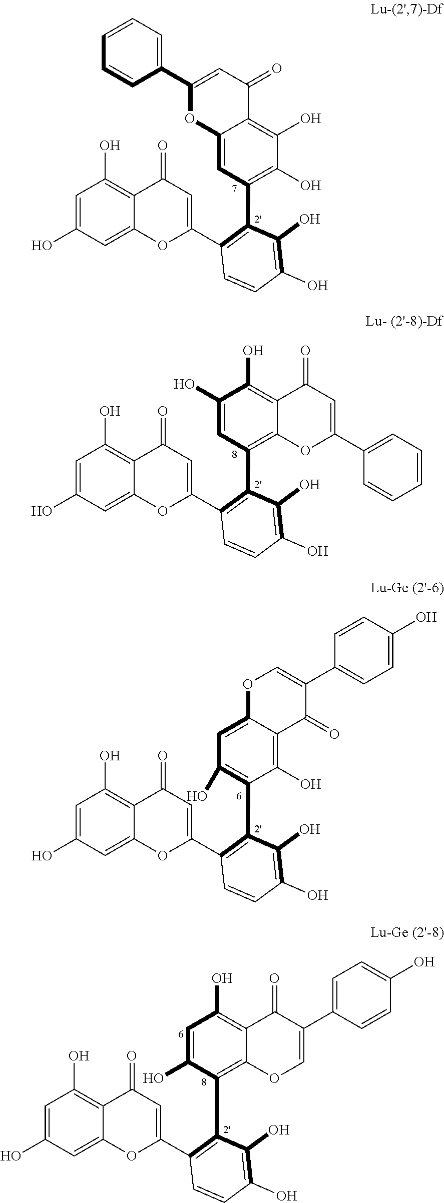

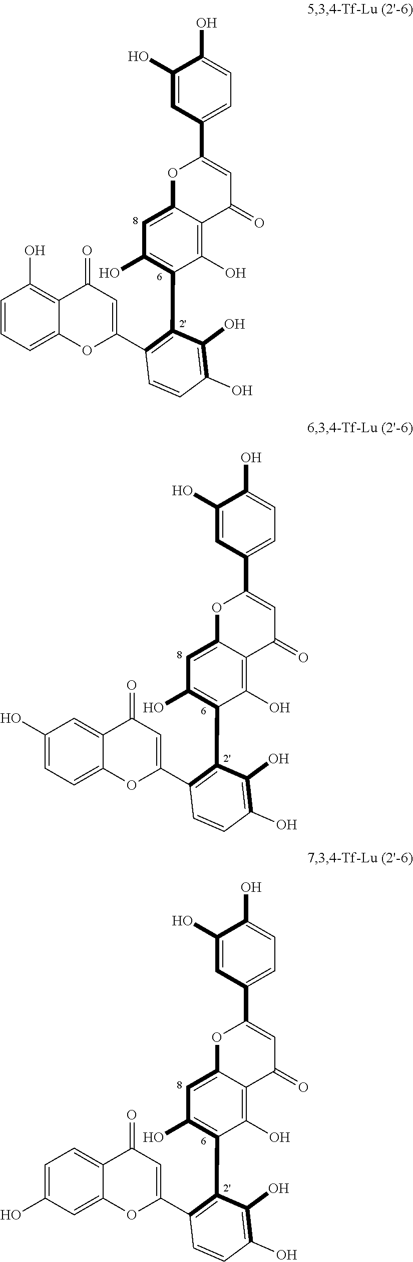

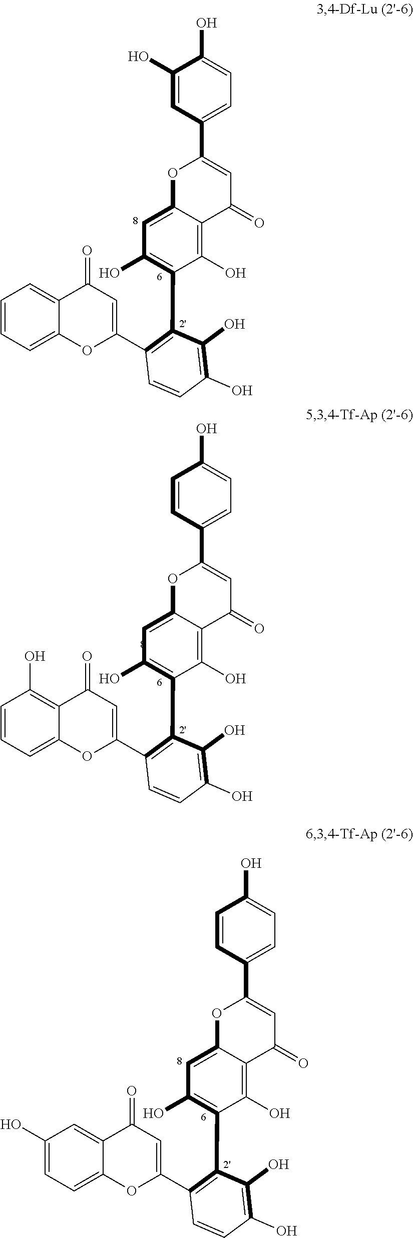

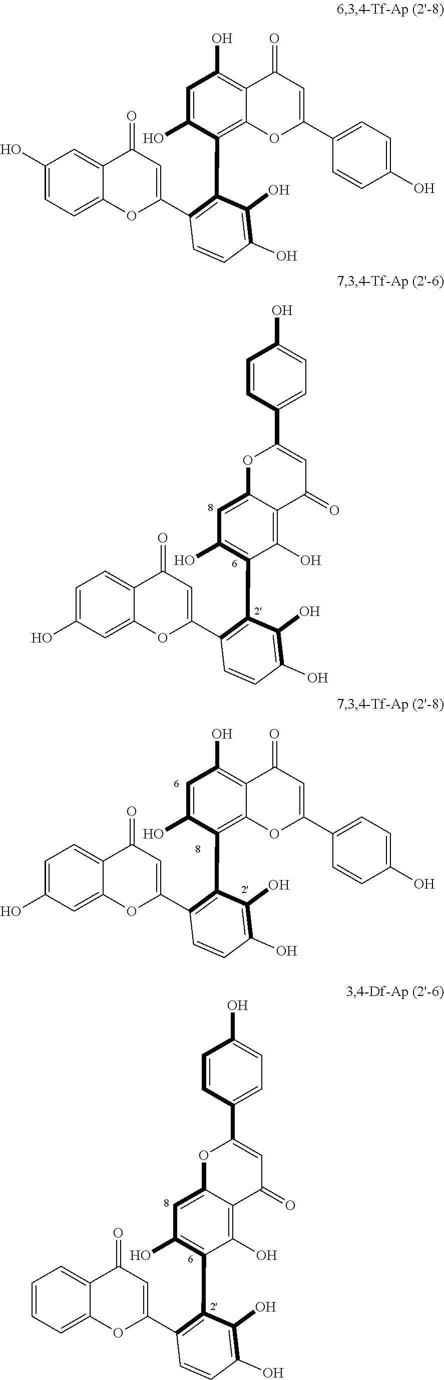

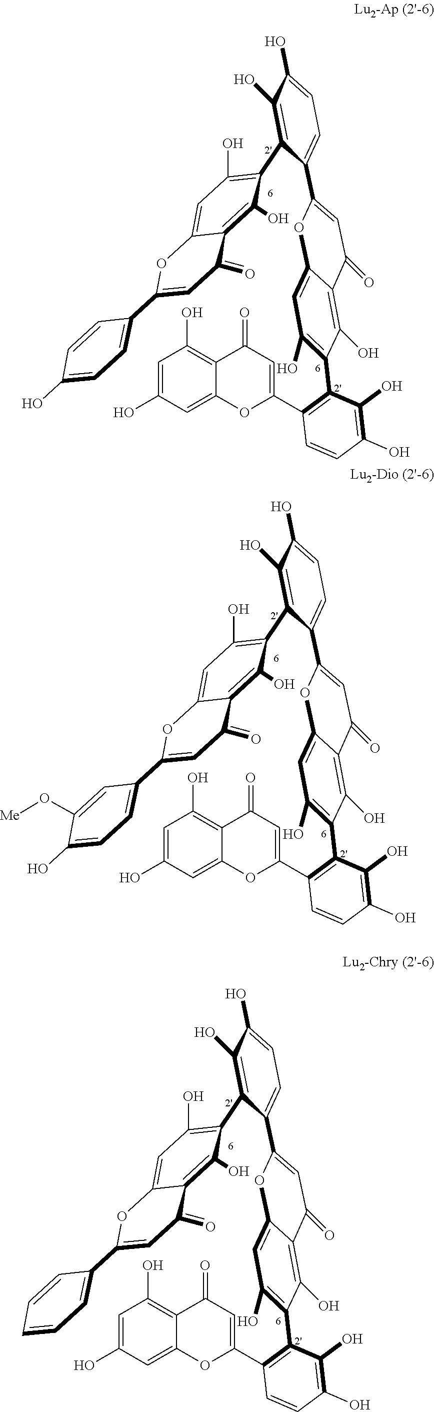

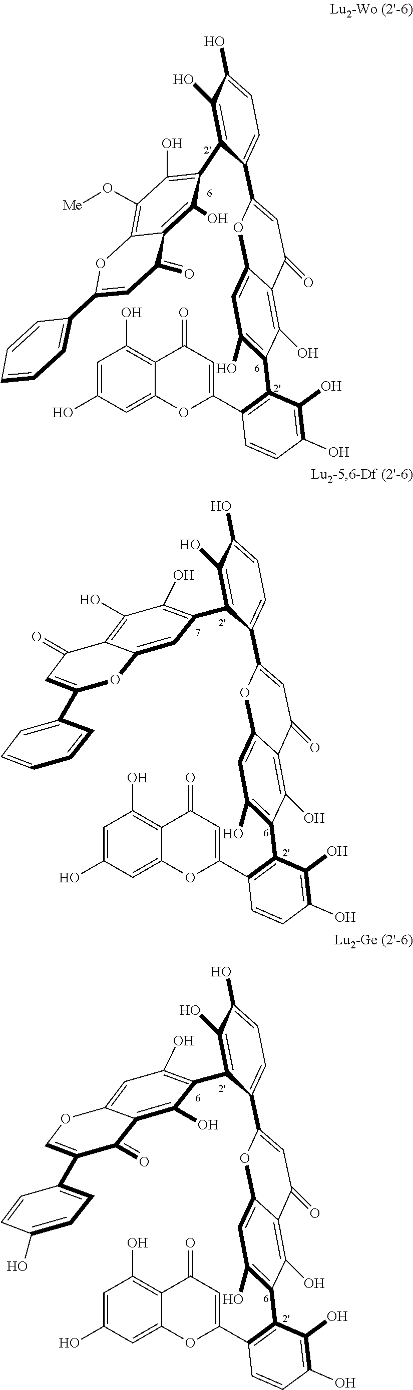

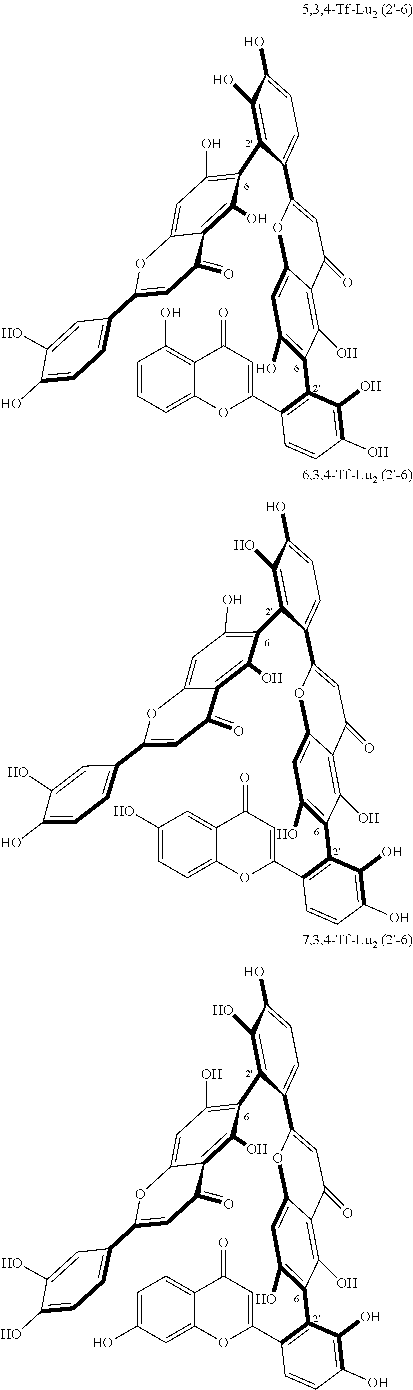

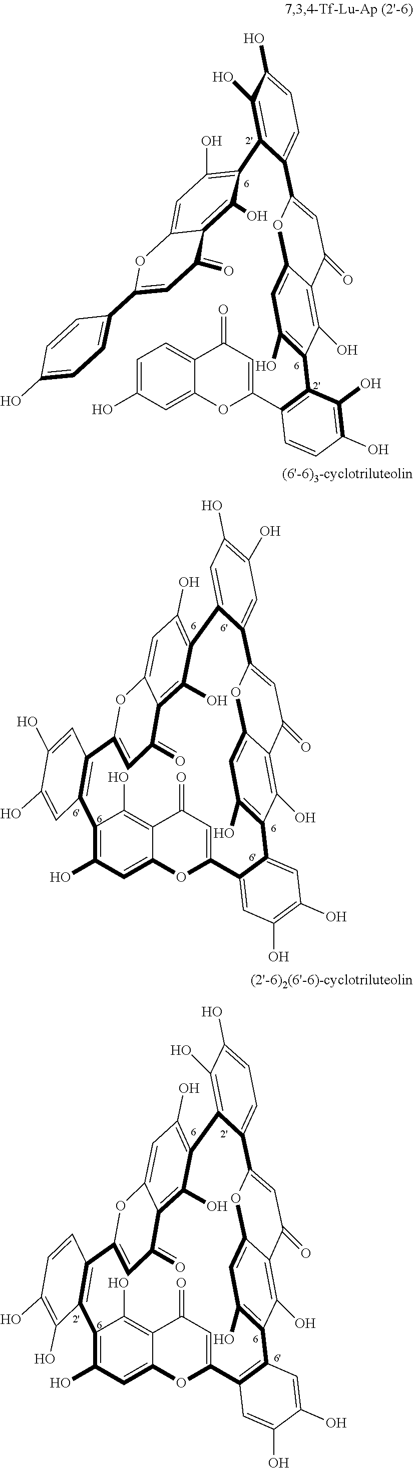

and pharmaceutically acceptable salts thereof.

The compound may also be a compound selected from the group consisting of:

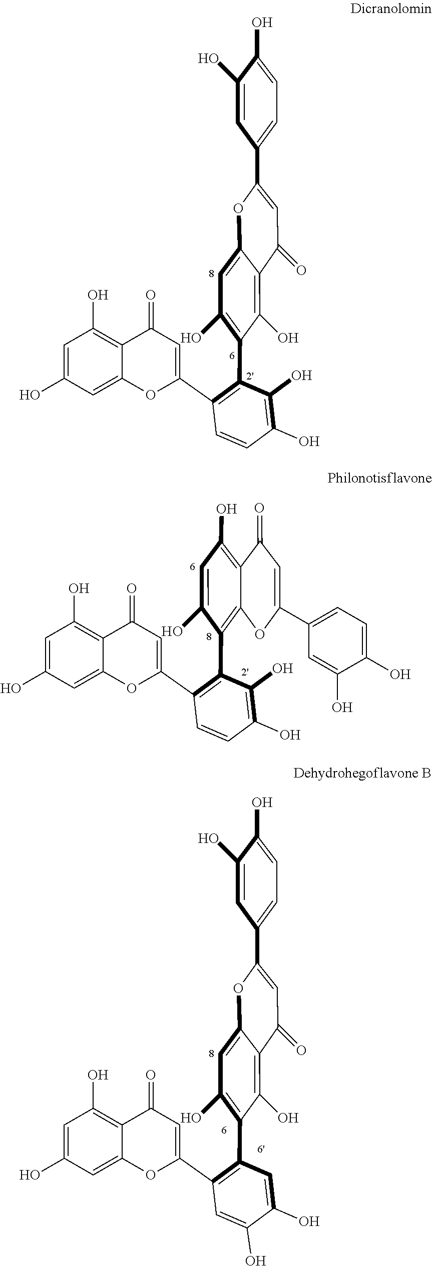

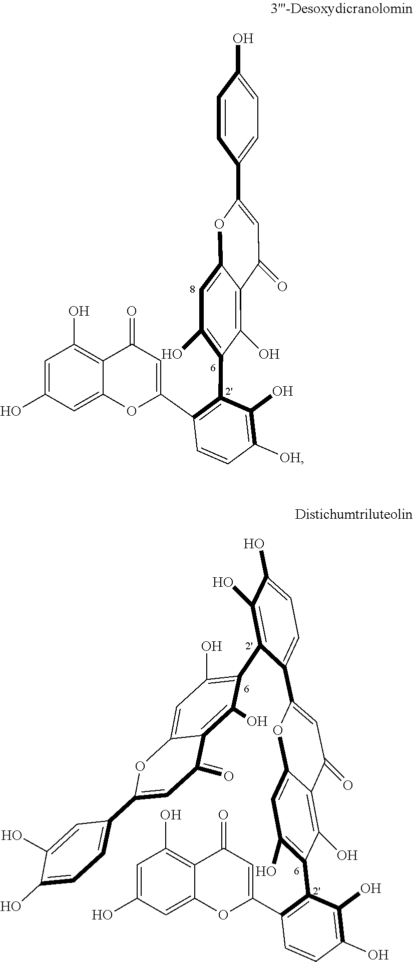

and pharmaceutically acceptable salts thereof.

In specific embodiments of the above medical uses (1), (2) and (3) according to the invention, the compound may be selected from dicranolamin, 3′″-Desoxydicranolomin, distichumtriluteolin, and pharmaceutically acceptable salts thereof. For example, the compound may be selected from dicranolamin, 3′″-Desoxydicranolomin, and pharmaceutically acceptable salts thereof. The compound may be dicranolamin or a pharmaceutically acceptable salt thereof.

The term “disorder or condition ameliorated by the inhibition of starch hydrolase” will be understood by those skilled in the art to include hyperglycaemia, diabetes and obesity.

Particular disorders or conditions that may be mentioned in relation to the aspects of the invention described hereinbefore include fungal infections (e.g. candida infection), hyperglycaemia, diabetes and obesity.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

For the avoidance of doubt, references herein (in any aspect or embodiment of the invention) to flavonoid-containing compounds includes references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound disclosed herein with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound disclosed herein in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by flavonoid-containing compounds are any solvates of the compounds and their salts. Preferred solvates are solvates formed by the incorporation into the solid state structure (e.g. crystal structure) of the compounds of the invention of compounds of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

“Pharmaceutically functional derivatives” of flavonoid-containing compounds as defined herein includes ester derivatives and/or derivatives that have, or provide for, the same biological function and/or activity as any relevant compound of the invention. Thus, for the purposes of this invention, the term also includes prodrugs of compounds of the invention. A particular example of prodrugs of compounds of the invention that comprise at least one hydroxy group is an ester derivative, such as an acetate ester derivative.

Nevertheless, the term “prodrug” of a relevant compound disclosed herein includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of compounds disclosed herein may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds disclosed herein wherein a hydroxyl, amino, sulfhydryl, carboxyl or carbonyl group in a compound disclosed herein is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. 1-92, Elsevier, New York-Oxford (1985).

Compounds disclosed herein, as well as pharmaceutically acceptable salts, solvates and pharmaceutically functional derivatives of such compounds are, for the sake of brevity, hereinafter referred to together as the “compounds disclosed herein”, or “compounds of the invention”.

Compounds disclosed herein may contain exocyclic double bonds and may thus exist as E (entgegen) and Z (zusammen) geometric isomers about each individual double bond. All such isomers and mixtures thereof are included within the scope of the invention.

Compounds disclosed herein may exist as regioisomers and may also exhibit tautomerism. All tautomeric forms and mixtures thereof are included within the scope of the invention.

Compounds disclosed herein may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

The term “glycosyl”, when used herein, refers to a group obtained by removing the hemiacetal hydroxyl group from the cyclic form of a monosaccharide, disaccharide, trisaccharide or oligosaccharide comprising 6 or fewer monosaccharide units. In particular cases, a glycosyl group may refer to a group obtained by removing the hemiacetal hydroxyl group from the cyclic form of a monosaccharide or disaccharide.

Unless otherwise stated, the term “alkyl” refers to an unbranched or branched, acyclic or cyclic, saturated or hydrocarbyl radical, which may be substituted or unsubstituted (with, for example, one or more halo atoms). Examples of “alkyl” groups include methyl, ethyl, propyl, (e.g. n-propyl or isopropyl), butyl (e.g. branched or unbranched butyl), pentyl, such as methyl. Where the term “alkyl” is a cyclic group (which may be where the group “cycloalkyl” is specified), it is preferably C₃₋₆ cycloalkyl and, more preferably, C₅₋₆ cycloalkyl.

The term “fluoroalkyl” refers to an alkyl group as defined above, where at least one hydrogen atom is replaced by a fluorine atom. In particular examples, a fluoroalkyl group may comprise from one to ten fluorine atoms, such as one to six, one to five, one to four, one to three, one to two, or one.

Further embodiments of the invention that may be mentioned include those in which the compounds disclosed herein are isotopically labelled. However, other, particular embodiments of the invention that may be mentioned include those in which the compounds disclosed herein are not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of formula I that are isotopically enriched at one or more positions in the compound.

The isotopic labelling or enrichment of the compound of formula I may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁸O, ³⁵S, ¹⁸F, ³⁷Cl, ⁷⁷Br, ⁸²Br and ¹²⁵I).

When the compound of formula I is labelled or enriched with a radioactive or nonradioactive isotope, compounds of formula I that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive.

The invention is illustrated by the below Examples, which are not to be construed as limitative.

### EXAMPLES

Materials

Sodium hydroxide, potassium hydroxide (KOH), lithium hydroxide, cesium hydroxide, potato Dextrose Agar (PDA) powder, dimethyl sulfoxide (DMSO) and ethanol were obtained from Merck & Co., Inc. Flavones (FL) including luteolin (Lu), apigenin (Ap), diosmetin (Dio), chrysin (Chry), wogonin (Wo), genistein (Ge), 5,6-dihydroxyflavone (“56”), 5,3′,4′-trihydroxyflavone (“534”), 6,3′,4′-trihydroxyflavone (“634”), 7,3′,4′-trihydroxyflavone (“734”) and 3′,4′-dihydroxyflavone (“34”) were obtained from Indofine Chemical Co., Inc., Hillsborough, NJ, USA. Hypochlorite acid (15%) was obtained from Merck & Co., Inc. Fluorescein disodium was obtained from Aldrich (Milwaukee, WI). Flavonoids compounds (7,8-dihydroxyflavone, baicalein, luteolin, scutellarein, fisetin, kaempferol, morin, myricetin, quercetin, 3,3′,4′-trihydroxyflavone, 3,5,7,8,3′,4′-hexahydroxyflavone, alpinetin, eriodictyol, liquiritigenin, hesperetin, naringenin, pinocembrin, ampelopsin, taxifolin, catechin, epicatechin, epigallocatechin) were obtained from Nanjing Plant Origin Biological Technology Co., Ltd. 96-well polystyrene microplates and the covers were purchased from VWR International Inc (Bridgeport, NJ). All aqueous solutions were prepared with 18.2 MΩ·cm ultrapure water obtained by a Millipore water purification system. Disodium tetraborate was obtained from Sigma.

Analytical Techniques

Nuclear Magnetic Resonance

¹H and ¹³C NMR spectra were measured using a Bruker AVANCE I 400 or 500 NMR spectrometer. Chemical shifts were reported in ppm from the solvent resonance as the internal standard (DMSO-d₆, δ=2.50). Spectra were reported as follows: chemical shift (δ ppm), multiplicity (s=singlet, d=doublet, t=triplet, q=quartet, hept=heptet, m=multiplet), coupling constants (Hz), integration and assignment. Chemical shifts were reported in ppm from the solvent resonance as the internal standard (DMSO, δ=39.52).

Mass Spectroscopy

High resolution mass spectrometry (HRMS) was performed on a Thermo Scientific LCQ Fleet ion trap mass spectrometer in ES negative mode.

Electron Paramagnetic Resonance (EPR) Spectroscopy

The EPR spectra were recorded on X-band EPR spectrometer (JES-TE100, JEOL, Tokyo, Japan), which was equipped with WIN-RAD EPR Data Analyzer System (Radical Research, Inc., Hino, Tokyo).

The flavonoids solutions (5.0 mM) were loaded in a capillary tube plugged with sealing putty (TERUMO CORPORATION, Tokyo Japan). Then, the capillary tube was put into an EPR tube (WILMAD QUARTZ (CFQ), DIAM. 5 mm), before placing in the TE mode cavity. The ESR experiments were performed at room temperature with the following parameters: microwave frequency: 9.19 GHz, microwave power: 1 mW, centre magnetic field: 328.348 mT, field sweep width: ±5 mT, sweep rate: 0.67 mT/min, time constant: 0.03, field modulation frequency: 500 kHz, and field modulation width: 0.025 mT. EPR data acquisition was controlled by the WIN-RAD EPR Data Analyzer System. The spectra were simulated by JEOL IsoSimu/Fa Version 2.2.0 isotropic simulation program.

Fluorescence

A Synergy HT microplate fluorescence reader (Bio-Tek Instruments, Inc., Winooski, VT) was used with fluorescence filters for an excitation wavelength of 485±20 nm and an emission wavelength of 530±25 nm. The plate reader was controlled by software KC4 3.0 (revision 29). Sample dilution was accomplished by a Precision X automatic pipetting system managed by precision power software (version 1.0) (Bio-Tek Instruments, Inc.).

Thin-Layer Chromatography (TLC) and Column Chromatography

Merck F254 silica gel-60 plates were used for thin-layer chromatography. Silica gel-60 (230-400 mesh) was selected as the solid phase for column chromatography.

HPLC and Liquid Chromatography-Tandem Mass Spectrometry (LC-MS/MS)

The HPLC system (Waters Arc HPLC System) was equipped with a C18 column (Luna 5 μm C18(2) 100A, LC Column 250×4.6 mm). The Waters 2998 Photodiode Array (PDA) Detector was connected to the HPLC system with detection wavelengths from 190 and 800 nm.

Bruker AmaZon-X was applied for LC-MS and LC-MS/MS. The LC-MS system was equipped with a C18 column (Phenomenex, Luna 5u C18, 250×4.6 mm) guard column (4×3.0 mm). All mass spectra were acquired in both positive and negative ion mode using electrospray ionization. The parent ion was selected with a width of ±2.5 Da and fragmented with 50% setting.

Semi-Prep HPLC

The semi-prep HPLC instrument (Waters semiprep HPLC system) was equipped with a C18 column (Phenomenex, (Luna 5 μM C18(2) 100A, LC Column 250×10 mm)) with a PDA detector.

X-Ray Crystallography

The frames were integrated with the Bruker SAINT software package using a narrow-frame algorithm. Data were corrected for absorption effects using the Multi-Scan method (SADABS).

Computational Studies

Geometry Optimization and Spin Density Distributions

Geometry optimization and spin density distributions were calculated using DFT at the UM062X/6-311+G(d,p) level of theory with Gaussian 09W software (M. Frisch et al., Gaussian 03, revision C. 02; Gaussian, Inc.: Wallingford, C T, 2004. The cube files for both the functions were generated using Multiwfn software (T. Lu & F. Chen, J. Comput. Chem. 2012, 33, 580-592) and isodensity surface plot for the non-covalent interaction studies were done using VMD visualization software (W. Humphrey, A. Dalke & K. Schulten, J. Mol. Graph. 1996, 14, 33-38). Molecular visualizations were created using CYLview (C. Legault, CYLview, 1.0b. Université de Sherbrooke 2009, 436, 437).

Gibbs Free Energies

The Gibbs free energies were calculated using DFT computations within the Gaussian 16 program (M. Frisch et al., Gaussian 03, revision C. 02; Gaussian, Inc.: Wallingford, C T, 2004.). Optimizations were done based on preliminary conformational searches with Schrödinger2 Maestro 10.6. The low-energy conformers that are with 5 kcal/mol of the global minimum were re-optimized at the level of M06-2X/6-31G(d) (Y. Zhao & D. G. Truhlar, Theor. Chem. Acc. 2008, 120, 215-241), with SMD (A. V. Marenich, C. J. Cramer & D. G. Truhlar, J. Phys. Chem. B 2009, 113, 6378-6396) solvation model for water. The vibrational frequency analyses were performed at the same level of theory to verify that minima have no imaginary frequencies and to evaluate its zero-point vibrational energy (ZPVE) and thermal corrections at 298 K. Single point energies were calculated using a larger basis set, 6-311+G(d,p), with the same solvation model.

General Procedure for the Characterization of Reaction Products with HPLC and LC-MS/MS

Isocratic elution method was applied for products analysis of luteolin homo-coupling. The LC-MS system was also applied in the analysis of the reaction products between flavonoids and HOCI. The samples (10 μL) were filtered through a 0.2 μm membrane (Merck Millipore, USA) before being injected into the HPLC system for analysis. Deionised (DI) water with 0.1% formic acid was selected as mobile phase A while ACN with 0.1% formic acid was applied for mobile phase B. The column was equilibrated with 71.5% mobile phase A for 10 min before isocratic elution of the same percentage mobile phase A from 0 to 35 min at a flow rate of 1.0 mL/min.

Bruker AmaZon-X was applied for LC-MS and LC-MS/MS to do characterization and fragments analysis of unknowns. The LC conditions for LC-MS analysis were similar to those mentioned above, except the detection wavelengths were 280 and 350 nm.

General procedure for the isolation of bioflavonoids using semi-prep HPLC The reaction solution was carefully acidified with 1 M HCl to neutral pH, then extracted with EA and purified by using the Semi-Prep HPLC instrument at a flow rate of 5 mL min⁻¹. The injection volume was 500 μL. Compound 1a was obtained at retention time 20.4 min with CH₃CN-water (45%:55%, 0.3% TFA v/v). Compound 5a was isolated at retention time 28.8 min eluted by CH₃CN-water (20%:80%, 0.3% TFA v/v). Compounds 8a and 8b were purified by CH₃CN-water (45%:55%, 0.3% TFA v/v) at retention time 36.6 min and 67.2 min, respectively. Compounds 10a, 10b and 10c were isolated with CH₃CN-water (35%:65%, 0.3% TFA v/v) at retention time 30.5 min, 42 min and 54 min, respectively. Compounds 12a and 12b were purified by CH₃CN-water (33%:67%, 0.3% TFA v/v), and were obtained at retention time 36.5 min and 53 min, respectively. The isolated yield was calculated from the mass obtained by semi-prep HPLC with C18 column.

### Example 1. Synthesis of Flavonoid Cross-Coupling Dimers

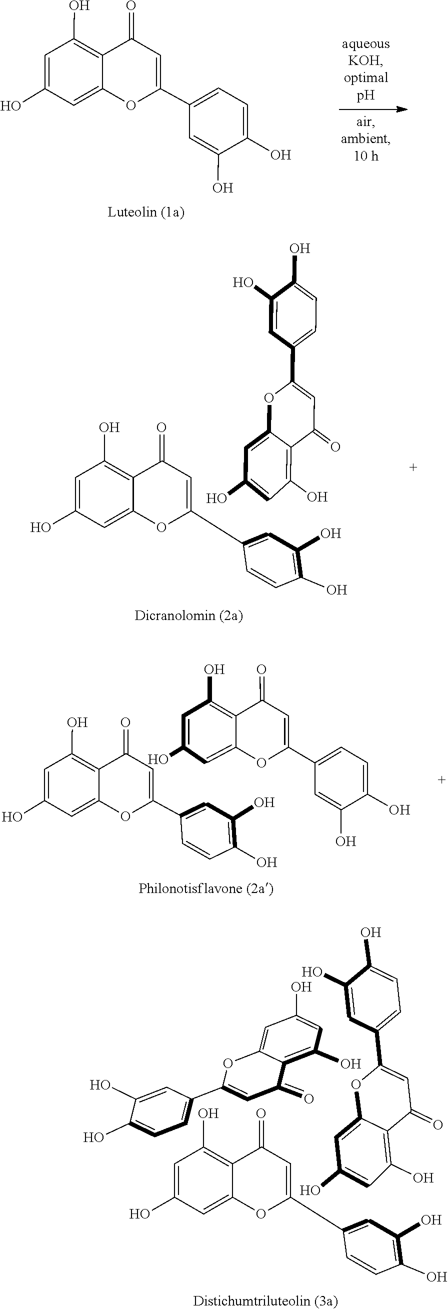

Luteolin (1a, 143 mg, 0.5 mmol) was added to an aqueous KOH solution (0.1 M, 30 mL) in a 50 mL centrifuge tube. The resulting solution had a pH value of 11.5. The tube was capped tightly to seal the reaction vessel and kept at room temperature for 10 hours without stirring. The resulting solution was then acidified with concentrated HCl (1.0 mL, 10 M) to give a solution with a pH value of 1˜ 2. Then, the resulting solution was extracted with EA (3×50.0 mL), and the organic layers were combined. Removal of the volatiles in vacuo resulted in a crude solid, which was purified over semi-prep HPLC with automatic fraction collection system to give pure 2a, 2a′, and 3a.

### 6-(6-(5,7-dihydroxy-4-oxo-4H-chromen-2-yl)-2,3-dihydroxyphenyl)-2-(3,4-dihydroxyphenyl)-5,7-dihydroxy-4H-chromen-4-one (Dicranolomin, 2a)

68 mg, 48% yield, brown solid. HPLC (ACN/Water=71.5:28.5, λ=300 nm), tᵣ(2a)=19.045 min, concentration of 2a=1.0 mM. ¹H NMR (500 MHz, DMSO-d₆) δ 13.15 (s, 1H), 12.79 (s, 1H), 10.73 (s, 1H), 10.68 (s, 1H), 10.13 (s, 1H), 9.91 (s, 1H), 9.42 (s, 1H), 8.45 (s, 1H), 7.48-7.40 (m, 2H), 7.19 (d, J=8.4 Hz, 1H), 6.95 (d, J=8.4 Hz, 1H), 6.90 (d, J=8.4 Hz, 1H), 6.70 (s, 1H), 6.54 (s, 1H), 6.09 (d, J=2.1 Hz, 1H), 6.06 (s, 1H), 5.97 (d, J=2.1 Hz, 1H). ¹³C NMR (126 MHz, DMSO) δ 182.12, 181.76, 166.95, 164.45, 164.09, 161.76, 159.24, 157.85, 156.77, 150.12, 148.86, 146.20, 144.86, 124.19, 121.99, 120.70, 120.28, 119.44, 116.52, 114.70, 113.78, 108.53, 106.69, 103.86, 103.77, 103.33, 99.14, 93.79, 93.73. HRMS (ESI-TOF) calcd for C₃₀H₁₈O₁₂=569.0725, found 569.0717.

Crystals of compound 2a suitable for X-ray analysis were obtained by slow evaporation from MeOH. A specimen of C₃₀H₁₈O₁₂, approximate dimensions 0.072 mm×0.123 mm×0.146 mm, was used for the X-ray crystallographic analysis. The X-ray intensity data were measured (λ=0.71073 Å). The total exposure time was 5.41 hours. The integration of the data using a triclinic unit cell yielded a total of 26930 reflections to a maximum θ angle of 28.31° (0.75 Å resolution), of which 7389 were independent (average redundancy 3.645, completeness=99.5%, Rᵢₙₜ=3.10%, R_{sig}=2.95%) and 5796 (78.44%) were greater than 2σ(F²). The final cell constants of a=8.4524(4) Å, b=10.0982(4) Å, c=18.4738(8) Å, α=95.392(2°), β=101.343(2°), γ=102.776(2°), volume=1492.08(11) Å³, are based upon the refinement of the XYZ-centroids of 9938 reflections above 20 σ(I) with 5.070°<2θ<56.58°. The ratio of minimum to maximum apparent transmission was 0.920. The calculated minimum and maximum transmission coefficients (based on crystal size) were 0.6862 and 0.7457. The structure was solved and refined using the Bruker SHELXTL Software Package, using the space group P−1, with Z=2 for the formula unit, C₃₀H₁₈O₁₂. The final anisotropic full-matrix least-squares refinement on F² with 543 variables converged at R1=6.80%, for the observed data and wR2=22.47% for all data. The goodness-of-fit was 0.848. The largest peak in the final difference electron density synthesis was 0.564 e⁻/Å³ and the largest hole was −0.685 e−/Å³ with an RMS deviation of 0.082 e⁻/Å³. On the basis of the final model, the calculated density was 1.515 g/cm³ and F(000), 712 e⁻. Crystallographic data have been deposited with the Cambridge Crystallograhic Data Centre (CCDC #2044714).

TABLE 1
Sample and crystal data for 2a.

| Identification code | J459 | |
|---|---|---|
| Chemical formula | C₃₀H₁₈O₁₂ | |
| Formula weight | 570.08 | g/mol |
| Temperature | 100(2) | K |
| Wavelength | 0.71073 | Å |
| Crystal size | 0.072 × 0.123 × 0.146 | mm |
| Crystal system | triclinic | |
| Space group | P −1 | |
| Unit cell dimensions | a = 8.4524(4) α = 95.392(2)° | |
| | b = 10.0982(4) Å β = 101.343(2)° | |
| | c = 18.4738(8) Å. γ = 102.776(2)° | |
| Volume | 1492.08(11) | Å³ |
| Z | 2 | |
| Density (calculated) | 1.515 | g/cm³ |
| Absorption coefficient | 0.120 | mm⁻¹ |

| | |
|---|---|
| F(000) | 712 |

TABLE 2
Data collection and structure refinement for 2a.

| | |
|---|---|
| Theta range for data collection | 2.27 to 28.31° |
| Index ranges | −11 <= h <= 11, −13 <= k <= 13, −23 <= l <= 24 |
| Reflections collected | 26930 |
| Independent reflections | 7389 [R(int) = 0.0310] |
| Coverage of independent reflections | 99.50% |
| Absorption correction | Multi-Scan |
| Max. and min. transmission | 0.7457 and 0.6862 |
| Structure solution technique | direct methods |
| Structure solution program | SHELXS-97 (Sheldrick 2008) |
| Refinement method | Full-matrix least-squares on F² |
| Refinement program | SHELXL-2017/1 (Sheldrick, 2017) |
| Function minimized | Σ w(Fₒ² − F_{c}²)² |
| Data/restraints/parameters | 7389/201/543 |
| Goodness-of-fit on F² | 0.848 |
| Final R indices | 5796 data; I > 2σ(I). R1 = 0.0680, wR2 = 0.2030 |
| | all data. R1 = 0.0842, wR2 = 0.2247 |
| Weighting scheme | w = 1/[σ²(Fₒ²) + (0.1602P)² + 2.3495P], where |
| | P = (Fo2 + 2Fc2)/3 |
| Largest diff. peak and hole | 0.564 and −0.685 eÅ⁻³ |
| R.M.S. deviation from mean | 0.082 eÅ⁻³ |

TABLE 3
Atomic coordinates and equivalent isotropic atomic displacement
parameters (Å2) for 2a. U(eq) is defined as one third
of the trace of the orthogonalized Uij tensor.

| | x/a | y/b | z/c | U(eq) |
|---|---|---|---|---|
| O1 | 0.6880(2) | 0.54829(17) | 0.93504(8) | 0.0261(4) |
| O2 | 0.3249(2) | 0.16316(17) | 0.57186(9) | 0.0270(4) |
| O3 | 0.8008(3) | 0.3373(2) | 0.15090(10) | 0.0365(4) |
| O4 | 0.9956(2) | 0.19908(19) | 0.94177(10) | 0.0309(4) |
| O5 | 0.9301(2) | 0.32086(18) | 0.82169(9) | 0.0296(4) |
| O6 | 0.4761(3) | 0.01630(19) | 0.76816(11) | 0.0399(5) |
| O7 | 0.3747(2) | 0.78006(18) | 0.67850(9) | 0.0321(4) |
| O8 | 0.3012(2) | 0.43405(18) | 0.79121(9) | 0.0307(4) |
| O9 | 0.6645(2) | 0.62360(18) | 0.64458(10) | 0.0318(4) |
| O10 | 0.6823(2) | 0.46963(18) | 0.52835(10) | 0.0313(4) |
| O11 | 0.9960(2) | 0.69871(19) | 0.46181(10) | 0.0330(4) |
| O12 | 0.0580(2) | 0.64909(18) | 0.32356(9) | 0.0301(4) |
| C1 | 0.7020(3) | 0.5660(2) | 0.86405(12) | 0.0255(5) |
| C2 | 0.7791(3) | 0.4907(3) | 0.82484(12) | 0.0270(5) |
| C3 | 0.8537(3) | 0.3889(2) | 0.85668(12) | 0.0249(5) |
| C4 | 0.8398(3) | 0.3721(2) | 0.93233(12) | 0.0239(4) |
| C5 | 0.9099(3) | 0.2785(2) | 0.97257(13) | 0.0260(5) |
| C6 | 0.8937(3) | 0.2675(3) | 0.04506(13) | 0.0282(5) |
| C7 | 0.8086(3) | 0.3507(3) | 0.07924(12) | 0.0277(5) |
| C8 | 0.7381(3) | 0.4435(3) | 0.04181(12) | 0.0268(5) |
| C9 | 0.7567(3) | 0.4534(2) | 0.96940(12) | 0.0244(4) |
| C10 | 0.6333(3) | 0.6796(2) | 0.83888(12) | 0.0268(5) |
| C11 | 0.6821(3) | 0.8062(3) | 0.88530(13) | 0.0334(5) |
| C12 | 0.6324(3) | 0.9183(3) | 0.86118(15) | 0.0359(6) |
| C13 | 0.5283(3) | 0.9057(3) | 0.79200(14) | 0.0311(5) |
| C14 | 0.4761(3) | 0.7790(2) | 0.74513(12) | 0.0269(5) |
| C15 | 0.5300(3) | 0.6652(2) | 0.76771(12) | 0.0244(4) |
| C16 | 0.4773(3) | 0.5326(2) | 0.71596(11) | 0.0234(4) |
| C17 | 0.3633(3) | 0.4188(2) | 0.72937(12) | 0.0245(4) |
| C18 | 0.3135(3) | 0.2942(2) | 0.68160(12) | 0.0260(5) |
| C19 | 0.3820(3) | 0.2849(2) | 0.61963(12) | 0.0238(4) |
| C20 | 0.5003(3) | 0.3925(2) | 0.60528(11) | 0.0242(4) |
| C21 | 0.5472(3) | 0.5179(2) | 0.65500(12) | 0.0241(4) |
| C22 | 0.5703(3) | 0.3755(2) | 0.54107(12) | 0.0255(4) |
| C23 | 0.5019(3) | 0.2476(2) | 0.49326(12) | 0.0275(5) |
| C24 | 0.3820(3) | 0.1487(2) | 0.50862(12) | 0.0256(5) |
| C25 | 0.2982(3) | 0.0175(2) | 0.46029(12) | 0.0268(5) |
| C26 | 0.1829(3) | 0.9182(2) | 0.48360(12) | 0.0273(5) |
| C27 | 0.1044(3) | 0.7944(2) | 0.43743(13) | 0.0266(5) |
| C28 | 0.1397(3) | 0.7713(2) | 0.36659(12) | 0.0256(5) |
| C29 | 0.2549(3) | 0.8695(3) | 0.34419(13) | 0.0311(5) |
| C30 | 0.3352(3) | 0.9917(3) | 0.39053(13) | 0.0316(5) |
| O1W | 0.8349(2) | 0.42752(19) | 0.41621(10) | 0.0330(4) |
| O1X | 0.2305(4) | 0.1955(2) | 0.83997(13) | 0.0556(6) |
| C1X | 0.3640(4) | 0.1948(3) | 0.90082(19) | 0.0507(8) |
| C2X | 0.3334(8) | 0.0640(5) | 0.9320(2) | 0.0876(18) |
| O1V | 0.0876(18) | 0.9899(15) | 0.7177(9) | 0.0700(10) |
| C1V | 0.964(3) | 0.8985(19) | 0.7423(16) | 0.0699(10) |
| C2V | 0.989(3) | 0.7675(17) | 0.7598(12) | 0.0700(10) |
| O1Y | 0.1185(8) | 0.9155(7) | 0.7888(4) | 0.0700(10) |
| C1Y | 0.9801(14) | 0.8532(12) | 0.7349(6) | 0.0700(10) |
| C2Y | 0.9990(13) | 0.8583(11) | 0.6584(5) | 0.0697(10) |
| O1U | 0.1232(11) | 0.9739(10) | 0.6781(6) | 0.0700(11) |
| C1U | 0.0305(17) | 0.9076(15) | 0.7117(7) | 0.0699(10) |
| C2U | 0.9353(17) | 0.8677(16) | 0.7651(8) | 0.0699(10) |

### 8-(6-(5,7-dihydroxy-4-oxo-4H-chromen-2-yl)-2,3-dihydroxyphenyl)-2-(3,4-dihydroxyphenyl)-5,7-dihydroxy-4H-chromen-4-one (Philonotisflavone, 2a′)

11 mg, 8% yield, brown solid. HPLC (ACN/Water=71.5:28.5, λ=300 nm), tᵣ(2a′)=30.572 min, concentration of 2a′=6.5 mM. ¹H NMR (500 MHz, DMSO-d₆) δ 13.15 (s, 2H), 10.72 (s, 2H), 10.15 (s, 4H), δ 7.59 (d, J=7.7 Hz, 2H), 7.20 (d, J=8.3 Hz, 1H), 7.01-6.89 (m, 3H), 6.61 (s, 1H), 6.11 (d, J=2.0 Hz, 1H), 6.05 (s, 1H), 5.99 (d, J=2.0 Hz, 1H). ¹³C NMR (75 MHz, DMSO) δ 182.24, 181.68, 166.78, 164.38, 163.88, 163.42, 162.15, 161.67, 159.14, 157.77, 156.72, 151.09, 148.70, 148.41, 144.63, 124.11, 121.90, 120.76, 120.66, 120.11, 116.15, 114.71, 110.57, 108.35, 106.67, 103.83, 103.76, 103.65, 99.07, 93.73. HRMS (ESI-TOF) calcd for C₃₀H₁₈O₁₂=569.0725, found 569.0715.

### 6-(6-(5,7-dihydroxy-4-oxo-4H-chromen-2-yl)-2,3-dihydroxyphenyl)-2-(2-(2-(3,4-dihydroxyphenyl)-5,7-dihydroxy-4-oxo-4H-chromen-6-yl)-3,4-dihydroxyphenyl)-5,7-dihydroxy-4H-chromen-4-one (Distichumtriluteolin, 3a)

90 mg, 42% yield, brown solid. HPLC (ACN/Water=71.5:28.5, λ=300 nm), tᵣ(3a)=22.742 min, concentration of 3a=10 mM. 3a=3a*+3a**. Atropisomer 3a*: ¹H NMR (500 MHz, Methanol-d₄) δ 7.41 (m, 2H), 7.27 (d, J=8.4 Hz, 1H), 7.19 (d, J=8.3 Hz, 1H), 7.00 (d, J=8.3 Hz, 1H), 6.95 (d, J=8.3 Hz, 1H), 6.59 (s, 1H), 6.58 (s, 1H), 6.57 (s, 1H), 6.23 (s, 1H), 6.17 (s, 1H), 6.13 (d, 1H), 6.04 (d, 1H), 6.01 (s, 1H). Atropisomer 3a**: ¹H NMR (500 MHz, Methanol-d₄) δ 7.41 (m, 2H), 7.27 (d, J=8.4 Hz, 1H), 7.19 (dd, J=8.3 Hz, 1H), 7.00 (d, J=8.3 Hz, 1H), 6.95 (d, J=8.3 Hz, 1H), 6.59 (s, 1H), 6.58 (s, 1H), 6.57 (s, 1H), 6.19 (s, 1H), 6.09 (s, 1H), 6.13 (d, 1H), 6.04 (d, 1H), 6.01 (s, 1H). ¹³C NMR (126 MHz, DMSO) δ 182.21, 181.81, 181.72, 166.66, 166.56, 164.15, 164.09, 162.28, 161.75, 159.07, 157.87, 157.06, 156.81, 150.04, 148.72, 146.17, 144.64, 124.14, 122.09, 120.63, 120.29, 119.43, 116.50, 114.75, 113.86, 108.37, 108.29, 106.66, 103.89, 103.75, 103.64, 103.43, 99.27, 93.96, 93.87, 93.73. HRMS (ESI-TOF) calcd for C₄₅H₂₇O₁₈=855.1192, found 855.1182.

Results and Discussion

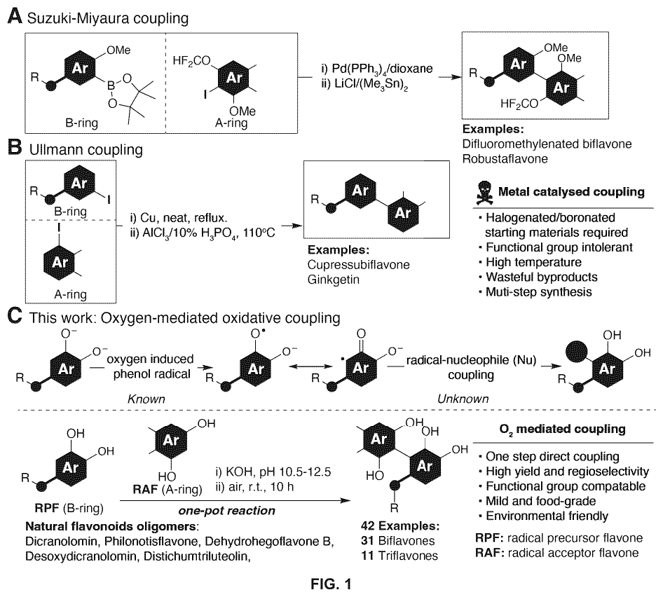

Hydroxyl group rich flavones (e.g. luteolin) are good reducing agents and have been well-known as potent dietary antioxidants in scavenging biologically relevant reactive oxygen species (N. Cotelle et al., Free Radic. Biol. Med. 1996, 20, 35-43). Moreover, under alkaline conditions, many weakly acidic flavones, including luteolin, undergo deprotonation to phenolates, which are sensitive to oxidation by molecular oxygen to their respective ortho-semiquinone radicals, as detected by ESR spectra (K. Kuwabara et al., Appl. Magn. Reson. 2018, 49, 911-924; and Ŝ. Rameŝová, R. S. J. Tarebek & I. Deganoca, Electrochim. Acta 2013, 110, 646-654). Nevertheless, the fates of these radicals were unknown. We envisioned that these electrons deficient semiquinone radicals may react with electron rich flavonoid anions by radical-nucleophile coupling. To verify this, we conducted HPLC analysis of the alkaline solution of luteolin (pH 11.5) and indeed found several products which were further characterized to be luteolin dimers and trimers by LC-MS. Herein, we report a novel catalyst-free oxidative coupling reaction of two sp² C—H bonds of flavones mediated by dissolved molecular oxygen as a hydrogen atom acceptor (FIG. 1).

TABLE 4
Condition evaluation of aerobic oxidative luteolin-luteolin cross-coupling^{a}

| | | | | | Yield of | | Yield |
|---|---|---|---|---|---|---|---|
| | | | | Conversion | 2a + 2a′ | Ratio of | of 3a |
| Entry | Base | pH | Atmosphere | (%) | (%)^{b} | (2a:2a′) | (%)^{b} |
| 1 | KOH | 11.5 | Air | 76 | 59 | 21:1 | 14 |
| 2 | Alkaline water | 11.5 | Air | 70 | 51 | 20:1 | 5 |
| | (Food grade) | | | | | | |
| 3 | KOH | 9.0 | Air | 25 | 22 | — | 0 |
| 4 | KOH | 9.5 | Air | 50 | 40 | 17:1 | 6 |
| 5 | KOH | 10.0 | Air | 53 | 45 | 13:1 | 8 |
| 6 | KOH | 10.5 | Air | 60 | 49 | 20:1 | 10 |
| 7 | KOH | 11.0 | Air | 69 | 52 | 22:1 | 13 |
| 8 | KOH | 12.0 | Air | 70 | 43 | 22:1 | 18 |
| 9 | KOH | 12.5 | Air | 58 | 37 | 17:1 | 12 |
| 10 | KOH | 13.0 | Air | 41 | 23 | 10:1 | 6 |
| 11 | KOH | 13.5 | Air | 26 | 10 | — | 0 |
| 12 | KOH | 14.0 | Air | 11 | 0 | — | 0 |
| 13 | LiOH | 11.5 | Air | 49 | 41 | 14:1 | 5 |
| 14 | NaOH | 11.5 | Air | 61 | 49 | 8:1 | 6 |
| 15 | CsOH | 11.5 | Air | 66 | 51 | 4:1 | 9 |
| 16 | Buffer | 11.5 | Air | 60 | 50 | 10:1 | 7 |
| 17 | KOH | 11.5 | Ar | 0 | 0 | — | 0 |
| 18 | KOH | 11.5 | O₂ | 100 | 0 | — | 0 |
| 19^{c} | KOH | 11.5 | Air | 85 | 52 | 15:1 | 12 |
| 20^{d} | KOH | 11.5 | Air | 71 | 55 | 20:1 | 11 |

^{a}The reaction condition: luteolin 1a (0.045 mmol) were dissolved in 3 mL base solution, incubated in sealed tube 15 mL without stirring.
^{b}Using HPLC to calculate conversion ratio, isocratic elution method (71.5% of mobile phase A: DI water with 0.1% formic acid and 21.5% mobile phase B: ACN with 0.1% formic acid) was applied for products analysis of luteolin-luteolin cross-coupling. Standard curves were built using isolated products.
^{c}At 10 g scale.
^{d}Reaction conducted in dark.

With the success of homo cross coupling of luteolin, we pondered whether a similar homo cross coupling reaction could be extended to other flavones. Thus, Ap, Dio, Chry, Wo, 5,6-dihydroxyflavone, and Ge were dissolved in alkaline water (pH 11.5). However, no desired coupling products were detected under the same condition. Instead, only starting materials were recovered. No free radical signals were detected by EPR spectroscopy in the reaction solution, suggesting that they are insensitive to oxygen. These flavones lack catecholic groups preventing them from forming ortho-semiquinone radical anions.

### Example 2. Gram-Scale Synthesis of Luteolin Coupling Products

By using the conditions in Example 1, the reaction was scaled up with 10 g of luteolin for the synthesis of 2a (42%, Lu-(2′-6)-Lu (This nomenclature was used to name the flavone dimers and oligomers. For example, Lu-(2′-6)-Lu represents luteolin (Lu) dimer linked by through the C(2′) of the first luteolin with the C(6) of the second luteolin), 2a′ (Lu-(2′-8)-Lu), 2a″ (Lu-(6′-6)-Lu) and 3a (Lu-(2′-6)-Lu-(2′-6)-Lu) in one-pot, where luteolin (10 g) was weighed and dissolved in KOH solution (0.05 M, 2.0 L) in four 1-liter plastic bottles with 500 mL per bottle, and the resulting solutions were neutralized with HCl (10 M) to give precipitates. The mixture was extracted with EA thrice (500 mL each). Then, the organic layers were combined and concentrated in vacuo to give the crude solid product, which was dissolved in methanol and purified over semi-prep HPLC with automatic fraction collection system to give pure 2a (4.2 g, 42%), 2a′ (0.12 g, 1.2%), 2a″ (0.10 g, 1.0%), and 3a (1.0 g, 10%).

### 6-(2-(5,7-dihydroxy-4-oxo-4H-chromen-2-yl)-4,5-dihydroxyphenyl)-2-(3,4-dihydroxyphenyl)-5,7-dihydroxy-4H-chromen-4-one (Dehydrohegoflavone B, Lu-(6′-6)-Lu, 2a″)

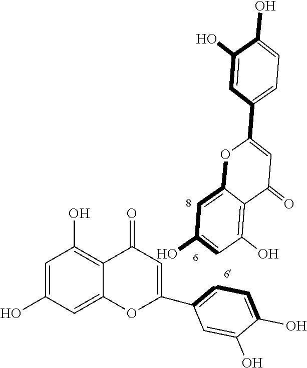

3 mg, 2% yield, brown solid. HPLC (ACN/Water=71.5:28.5, λ=300 nm), tᵣ(2a″)=15.922 min, concentration of 2a″=3.14 mM. ¹H NMR (500 MHz, DMSO-d₆) δ 13.07 (s, 1H), 12.72 (s, 1H), 10.14 (s, 3H), 9.62 (s, 3H), 7.29 (s, 1H), 7.12-6.97 (m, 2H), 6.83 (s, 1H), 6.76 (d, J=8.4 Hz, 1H), 6.61 (s, 1H), 6.31 (s, 1H), 6.08 (d, J=2.1 Hz, 1H), 5.98 (s, 1H), 5.81 (d, J=2.1 Hz, 1H). ¹³C NMR (126 MHz, DMSO) δ 182.29, 181.61, 166.41, 164.57, 164.29, 161.68, 160.65, 157.69, 154.31, 150.28, 148.75, 146.12, 145.54, 123.66, 123.48, 123.34, 121.80, 120.38, 119.04, 116.34, 116.06, 114.03, 107.51, 106.65, 103.91, 103.72, 102.88, 99.21, 98.93, 93.67. HRMS (ESI-TOF) calcd for C₃₀H₁₈O₁₂=569.0725, found 569.0727.

Results and Discussion

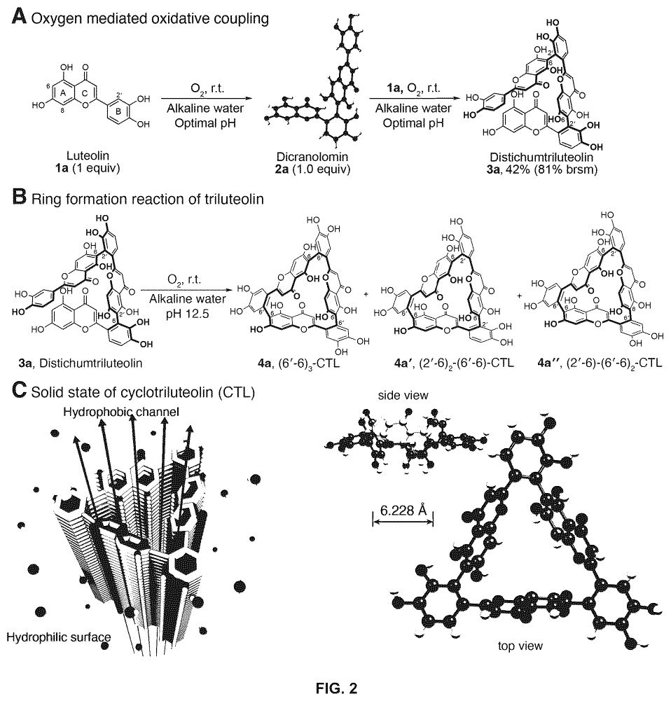

2a, 2a′, 2a″, and 3a were successfully synthesized in one-pot (FIG. 2). These compounds were originally isolated from moss, one of the oldest land plants, particularly Rhizogonium distichum which contains all four triluteolin regioisomers including bartramiatriluteolin, strictatriluteolin, and rhizogoniumtriluteolin (H. Geiger, and T. Seeger, Z. Naturforsch. 2000, 55c, 870-873). Their biosynthesis is likely mediated by enzymes (such as polyphenol oxidase) under the neutral physiological pH of moss. High contents of flavonoids in moss (as high as 10% of its dry weight) were suggested to protect the plant from biotic (e.g. fungi) and abiotic stress (temperature, water, reactive oxygen species, and ultraviolet (UV)-light) (H. Wang et al., Plant Sci. 2020, 298, 110591; and M. Waterman et al., J. Nat. Prod. 2017, 80, 2224-2231). Product 3a was characterized by HRMS, ¹H and ¹³C NMR spectra, which reveal the existence of atropisomers due to the hindered rotation of interflavonyl bonds (C. L. Covington et al., J. Nat. Prod. 2016, 79, 2530-2537). Such isomerism is common in complex natural products including tryptorubin A (S. H. Reisberg et al., Science 2020, 367, 458-463).

### Example 3. Syntheses of Cyclotriluteolins (CTLs)

Triluteolins such as 3a, have one B ring and one A ring on the terminal luteolin units, respectively, that are close to each other for intramolecular oxidative coupling (FIG. 2B).

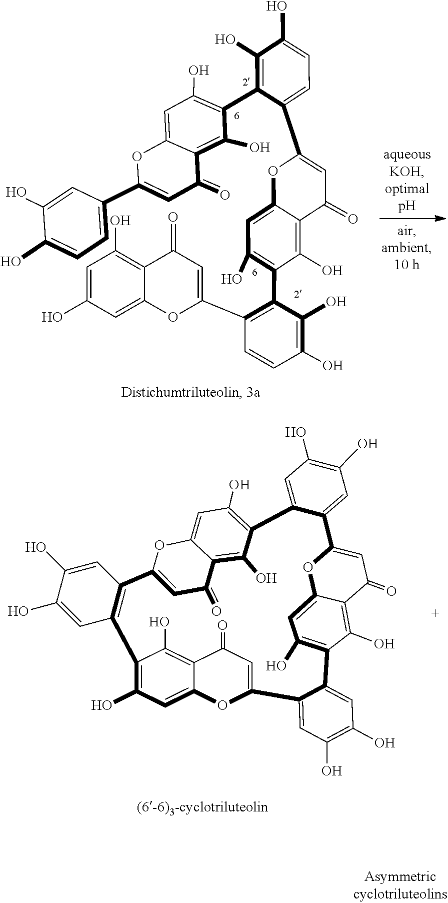

Cyclotriluetolins (e.g. 4) were prepared from trimers (e.g. 3a) (50 mg, 0.06 mmol) in an aqueous KOH solution (10 mL, 0.1 M) in a 50 mL centrifuge tube, by an analogous protocol to Example 1 except the pH of the reaction mixture was adjusted to 12.5 with concentrated KOH before the tube was capped tightly and kept at room temperature for 10 h without stirring (FIG. 2B).

### (6′-6)₃-cyclotriluteolin, ((6′-6)3-CTL, 4a)

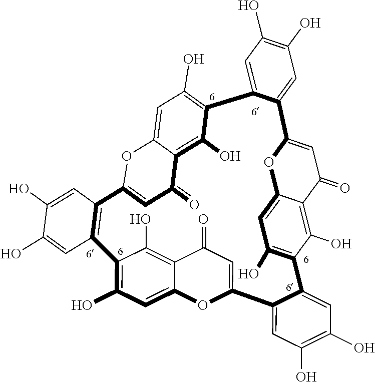

5 mg, 10% yield, white solid. HPLC (Luna 5 μm C18(2) 100 Å, LC Column 250×4.6 mm, ACN/Water=71.5:28.5, flow rate 1.0 mL/min, λ=300 nm), injection volume=10 μL, tᵣ(4a)=9.045 min. ¹H NMR (500 MHz, DMSO-d₆) δ 12.74 (s, 1H), 12.70 (s, 1H), 12.69 (s, 1H), 10.56 (s, 1H), 10.49 (s, 1H), 9.96 (s, 1H), 9.59 (s, 1H), 9.39 (s, 1H), 8.45 (s, 1H), 6.94 (s, 1H), 6.89 (d, J=2.4 Hz, 4H), 6.63 (s, 1H), 6.30-6.24 (m, 3H), 6.06 (d, J=11.1 Hz, 3H). ¹³C NMR (126 MHz, DMSO) δ 182.01, 181.98, 181.94, 168.03, 167.97, 167.67, 163.55, 163.46, 162.95, 158.62, 158.49, 158.46, 156.90, 156.67, 148.23, 148.19, 147.99, 145.20, 145.10, 145.00, 126.42, 126.38, 125.58, 123.87, 120.74, 120.60, 119.90, 119.79, 119.64, 116.96, 114.68, 111.40, 107.94, 107.80, 103.27, 103.10, 93.27. HRMS (ESI-TOF) calcd for C₄₅H₂₃O₁₈=851.0890, found 851.0901.

### (2′-6)2(6′-6)-cyclotriluteolin, ((2′-6)2(6′-6)-CTL, 4b)

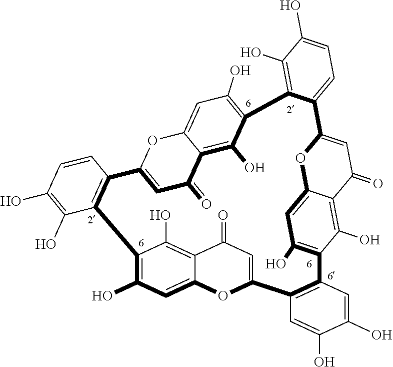

4b was obtained by crystallisation of 4a. ¹H NMR (500 MHz, DMSO-d₆) δ 14.73 (s, 1H), 13.17 (s, 1H), 11.93 (s, 1H), 10.69 (s, 1H), 10.54 (s, 1H), 10.24 (s, 1H), 9.98 (s, 1H), 9.69 (s, 1H), 9.43 (s, 1H), 8.43 (d, J=12.2 Hz, 3H), 8.20 (s, 1H), 7.24 (d, J=8.5 Hz, 1H), 7.09 (s, 1H), 6.95-6.84 (m, 4H), 6.59 (s, 1H), 6.22 (s, 1H), 5.98 (s, 1H), 5.72 (s, 1H), 5.26 (s, 1H). ¹³C NMR (126 MHz, DMSO) δ 195.02, 183.13, 182.47, 169.26, 168.37, 165.97, 161.10, 160.11, 159.56, 158.24, 157.75, 157.01, 154.98, 154.58, 149.43, 148.15, 147.17, 145.73, 144.97, 144.74, 126.59, 123.16, 121.80, 121.47, 120.83, 120.08, 120.02, 118.56, 114.75, 114.63, 114.05, 112.68, 108.33, 108.29, 106.39, 106.36, 105.94, 105.81, 103.73, 102.11, 101.15, 95.19, 95.03, 92.67. HRMS (ESI-TOF) calcd for C₄₅H₂₃O₁₈=851.0890, found 851.0882.

Results and Discussion

By dissolving 3a in alkaline water (pH 12.5) at room temperature overnight, three major cyclotriluteolins, 4a, 4a′, and 4a″ (FIG. 2B), were formed together with some luteolin monomer and 2a were observed by HPLC in the reaction mixture. Apparently, interflavonyl bond isomerization had occurred under the reaction conditions and the expected (2-6)3-triluteolin isomer was not detected. The interflavonyl bond cleavage would explain the formation of 1a and 2a. These cyclotriluteolins are regioisomers of naturally occurring cyclobartramiatriluteolin ((2′-8)₃ interflavonyl bonds) isolated from moss (H. Geiger et al., Phytochemistry 1995, 39, 465-467).

### Example 4. Characterisation of 4a

To confirm the structure of 4a prepared in Example 3, single crystals were grown from its methanolic solution and the molecular structure was determined.

Results and Discussion

Crystals of compound 4a suitable for X-ray analysis were obtained by slow evaporation from MeOH. A specimen of C₄₅H₂₄O₁₈, approximate dimensions 0.248×0.247×0.168 mm, was used for the X-ray crystallographic analysis. The X-ray intensity data were measured (λ=1.54178 Å). The integration of the data using a triclinic unit cell yielded a total of 33495 reflections. The final cell constants of a=10.4383(7) Å, b=16.9456(11) Å, c=18.4841(12) Å, α=116.927(3°), β=97.234(3°), γ=100.043(3°), volume=2791.1(3) Å3, are based upon the refinement of the XYZ-centroids of 4286 reflections above 20 σ(I) with 8.870°<2θ<133.1°. The calculated minimum and maximum transmission coefficients (based on crystal size) were 0.5865 and 0.7528. The structure was solved and refined using the Bruker SHELXTL Software Package, using the space group P −1, with Z=2 for the formula unit, C₄₅H₂₄O₁₈. The final anisotropic full-matrix least-squares refinement on F² at R1=10.5%, for the observed data and wR2=30.28% for all data. The goodness-of-fit was 1.062. The largest peak in the final difference electron density synthesis was 1.044 e−/Å³ and the largest hole was −0.668 e−/Å³. On the basis of the final model, the calculated density was 1.285 g/cm³ and F(000), 1103 e⁻. Crystallographic data have been deposited with the Cambridge Crystallograhic Data Centre (CCDC #2044716).

TABLE 5
Sample and crystal data for 4a.

| Identification code | J552 | |
|---|---|---|
| Chemical formula | C₄₅H₂₄O₁₈ | |
| Formula weight | 852.10 | g/mol |
| Temperature | 100(2) | K |
| Wavelength | 1.54178 | Å |
| Crystal size | 0.248 × 0.247 × 0.168 | mm |
| Crystal system | triclinic | |
| Space group | P −1 | |
| Unit cell dimensions | a = 10.4383(7) Å. α = 116.927(3)° | |
| | b = 16.9456(11) Å. β = 97.234(3)° | |
| | c = 18.4841(12) Å. γ = 100.043(3)° | |
| Volume | 2791.1(3) | Å³ |
| Z | 1 | |
| Density (calculated) | 1.285 | g/cm³ |
| Absorption coefficient | 0.948 | mm⁻¹ |

| | |
|---|---|
| F(000) | 1103 |

TABLE S6
Data collection and structure refinement for 4a.

| | |
|---|---|
| Theta range for data collection | 2.943 to 66.885° |
| Index ranges | −12 <= h <= 12, −20 <= k <= 20, −19 <= l <= 22 |
| Reflections collected | 33495 |
| Coverage of independent | 98.8% |
| reflections | |
| Absorption correction | Multi-Scan |
| Max. and min. transmission | 0.7528 and 0.5865 |
| Structure solution technique | direct methods |
| Structure solution program | SHELXS-97 (Sheldrick 2008) |
| Refinement method | Full-matrix least-squares on F² |
| Refinement program | SHELXL-2017/1 (Sheldrick, 2017) |
| Function minimized | Σ w(Fₒ² − F_{c}²)² |
| Data/restraints/parameters | 9812/13/747 |
| Goodness-of-fit on F² | 1.062 |
| Final R indices | R1 = 0.1004, wR2 = 0.2964 |
| | all data. R1 = 0.1050, wR2 = 0.3028 |
| Weighting scheme | w = 1/[σ²(Fₒ²) + (0.1943P)²], where P = (Fo2 + 2Fc2)/3 |
| Largest diff. peak and hole | 1.044 and −0.668 eÅ⁻³ |

TABLE S7
Atomic coordinates and equivalent isotropic atomic displacement
parameters (Å2) for 4a. U(eq) is defined as one third
of the trace of the orthogonalized Uij tensor.

| | x/a | y/b | z/c | U(eq) |
|---|---|---|---|---|
| O(1) | 11874(3) | 9106(2) | 10093(2) | 52(1) |
| O(2) | 10107(4) | 7717(3) | 10002(2) | 44(1) |
| O(2A) | 12606(11) | 9018(5) | 8741(5) | 57(3) |
| O(3) | 11045(3) | 5600(2) | 8908(2) | 49(1) |
| O(4) | 7141(3) | 6349(2) | 8171(2) | 45(1) |
| O(5) | 6727(3) | 3711(2) | 8485(2) | 42(1) |
| O(6) | 10779(3) | 4212(2) | 9179(2) | 66(1) |
| O(7) | 3241(4) | 434(3) | 8615(3) | 80(1) |
| O(8) | 3141(5) | 730(3) | 7349(3) | 53(2) |
| O(8A) | 5243(10) | 1234(10) | 9811(7) | 81(4) |
| O(9) | 6472(3) | 906(2) | 6787(2) | 44(1) |
| O(10) | 3815(3) | 3000(2) | 7527(2) | 56(1) |
| O(11) | 5039(3) | 2037(2) | 4950(2) | 42(1) |
| O(12) | 7051(3) | 350(2) | 5343(2) | 51(1) |
| O(13) | 4761(3) | 2009(2) | 1558(2) | 49(1) |
| O(14) | 5920(3) | 3431(2) | 3042(2) | 39(1) |
| O(14A) | 3970(13) | 358(7) | 1424(6) | 50(4) |
| O(15) | 8611(2) | 3013(2) | 4211(2) | 37(1) |
| O(16) | 4556(2) | 3683(2) | 4786(2) | 48(1) |
| O(17) | 8581(2) | 5609(2) | 6778(1) | 35(1) |
| O(18) | 10800(2) | 4184(2) | 5246(2) | 43(1) |
| C(1) | 10439(3) | 6710(2) | 7830(2) | 33(1) |
| C(2) | 11393(4) | 7440(2) | 7900(2) | 39(1) |
| C(3) | 11869(4) | 8243(2) | 8660(2) | 43(1) |
| C(4) | 11399(4) | 8311(2) | 9349(2) | 40(1) |
| C(5) | 10467(4) | 7577(2) | 9283(2) | 37(1) |
| C(6) | 9970(3) | 6772(2) | 8529(2) | 34(1) |
| C(7) | 9094(3) | 5962(2) | 8508(2) | 34(1) |
| C(8) | 9706(3) | 5405(2) | 8717(2) | 38(1) |
| C(9) | 8932(4) | 4631(2) | 8723(2) | 39(1) |
| C(10) | 7544(4) | 4451(2) | 8500(2) | 38(1) |
| C(11) | 6905(4) | 4990(2) | 8291(2) | 41(1) |
| C(12) | 7691(4) | 5764(2) | 8319(2) | 38(1) |
| C(13) | 9524(4) | 4050(3) | 8957(3) | 48(1) |
| C(14) | 8606(4) | 3295(3) | 8930(3) | 50(1) |
| C(15) | 7276(4) | 3148(3) | 8693(2) | 43(1) |
| C(16) | 6231(4) | 2409(3) | 8645(3) | 48(1) |
| C(17) | 6326(5) | 2246(4) | 9329(3) | 61(1) |
| C(18) | 5329(6) | 1589(4) | 9322(4) | 70(2) |
| C(19) | 4251(5) | 1088(4) | 8650(4) | 65(1) |
| C(20) | 4180(5) | 1235(3) | 7960(3) | 53(1) |
| C(21) | 5160(4) | 1886(3) | 7948(3) | 44(1) |
| C(22) | 5136(4) | 1946(2) | 7166(2) | 41(1) |
| C(23) | 5806(4) | 1429(2) | 6599(2) | 38(1) |
| C(24) | 5790(3) | 1431(2) | 5835(2) | 37(1) |
| C(25) | 5105(4) | 1998(2) | 5679(2) | 39(1) |
| C(26) | 4456(4) | 2539(3) | 6236(2) | 46(1) |
| C(27) | 4464(4) | 2503(3) | 6976(3) | 46(1) |
| C(28) | 6421(4) | 865(2) | 5218(2) | 39(1) |
| C(29) | 6298(4) | 940(2) | 4472(2) | 42(1) |
| C(30) | 5636(3) | 1506(2) | 4365(2) | 38(1) |
| C(31) | 5434(3) | 1632(2) | 3623(2) | 38(1) |
| C(32) | 4821(4) | 875(2) | 2844(2) | 42(1) |
| C(33) | 4588(4) | 985(3) | 2145(2) | 43(1) |
| C(34) | 4955(4) | 1843(2) | 2210(2) | 39(1) |
| C(35) | 5581(3) | 2601(2) | 2993(2) | 37(1) |
| C(36) | 5829(3) | 2510(2) | 3698(2) | 34(1) |
| C(37) | 6573(3) | 3332(2) | 4512(2) | 34(1) |
| C(38) | 7949(3) | 3555(2) | 4735(2) | 31(1) |
| C(39) | 8677(3) | 4338(2) | 5495(2) | 31(1) |
| C(40) | 7949(3) | 4861(2) | 6022(2) | 32(1) |
| C(41) | 6572(3) | 4659(2) | 5819(2) | 38(1) |
| C(42) | 5894(3) | 3906(2) | 5049(2) | 38(1) |
| C(43) | 10120(3) | 4621(2) | 5724(2) | 35(1) |
| C(44) | 10706(3) | 5429(2) | 6518(2) | 36(1) |
| C(45) | 9942(3) | 5877(2) | 7009(2) | 33(1) |
| O(1S) | 2333(9) | 2199(6) | 3864(6) | 86(2) |
| C(1S) | 1846(14) | 2594(10) | 3409(10) | 90(3) |
| O(2S) | 1856(19) | 2257(13) | 2840(13) | 90(5) |
| C(2S) | 840(20) | 1335(14) | 2365(13) | 63(4) |
| O(3S) | 2902(3) | 739(2) | 208(2) | 50(1) |
| C(3S) | 1816(7) | 1122(5) | 230(4) | 85(2) |
| O(4S) | 5564(4) | 3991(3) | 1871(2) | 71(1) |
| C(4S) | 5855(9) | 3575(6) | 1124(6) | 112(2) |
| O(5S) | 267(7) | 3286(5) | 6437(5) | 92(2) |
| O(5A) | 324(17) | 3314(12) | 6990(12) | 96(3) |
| C(5S) | −1042(8) | 3122(6) | 6579(5) | 98(2) |
| O(6S) | 11230(20) | 9331(14) | 12045(13) | 99(5) |
| C(6S) | 10670(20) | 8982(18) | 11288(15) | 75(6) |
| O(7S) | 8204(9) | −706(6) | 4129(5) | 85(2) |
| C(7S) | 9070(15) | −483(10) | 4827(9) | 93(4) |
| O(8S) | 8262(16) | 7019(12) | 10545(11) | 59(4) |
| O(9S) | 9180(30) | −1900(18) | 5145(18) | 100(7) |
| O(10S) | 8960(20) | −1983(15) | 4578(15) | 101(5) |
| O(11S) | 9640(30) | −1682(17) | 5322(16) | 76(6) |
| O(12S) | 2839(4) | 4235(3) | 7186(2) | 72(1) |
| O(13S) | 3405(3) | 4686(2) | 5909(2) | 46(1) |
| O(14S) | 1774(8) | −1058(5) | 7121(5) | 66(2) |
| O(15S) | 5341(18) | 6117(13) | 9708(12) | 89(5) |
| O(16S) | 1466(8) | −1008(5) | 6856(5) | 69(2) |
| O(17S) | 8067(6) | 6764(5) | 10267(4) | 41(2) |
| O(18S) | 9540(50) | 8290(30) | 12610(30) | 113(13) |
| O(19S) | 9370(20) | 8516(16) | 11864(15) | 55(5) |
| O(20S) | 7466(16) | 7293(10) | 10773(10) | 64(3) |
| O(21S) | 8250(20) | 7516(15) | 11147(14) | 45(5) |
| O(22S) | 7250(30) | 6907(19) | 11167(17) | 64(6) |
| O(23S) | 11954(13) | 9138(9) | 11645(8) | 58(3) |
| O(24S) | 11102(8) | 9622(6) | 11481(5) | 79(2) |
| O(25S) | 6840(30) | 6790(20) | 10430(20) | 74(7) |

The ORTEP plot (FIG. 2C) shows the structure to be 4a′ instead of the expected 4a. The structure of 4a′ adopts a triangular shape with each corner occupied by B rings of the luteolin and the three edges were fenced by the benzopyranyl moieties (with a length of 7.845 Å), forming a hydrophobic cavity with an opening of ˜ 6.228 Å. The C(4)=O and C(5)-OH form intramolecular hydrogen bond and the benzopyranyl plane tilts with a dihedral angle of about 70° with the plane coincident with the paper surface. In the solid state, 4a′ molecules form hydrophobic channels with hydrophilic OH groups (C(7)OH, C(3′)-OH and C(4′)-OH) pointing outward and C(4)=O and C(5)-OH edge pointing inward. With its unique shape and phenolic groups, cyclotriluteolins may complex guest molecules and metal ions. Thus, it is an intriguing building block for the construction of a functional covalent organic framework (COF).

Isomerization of Cyclotriluteolin

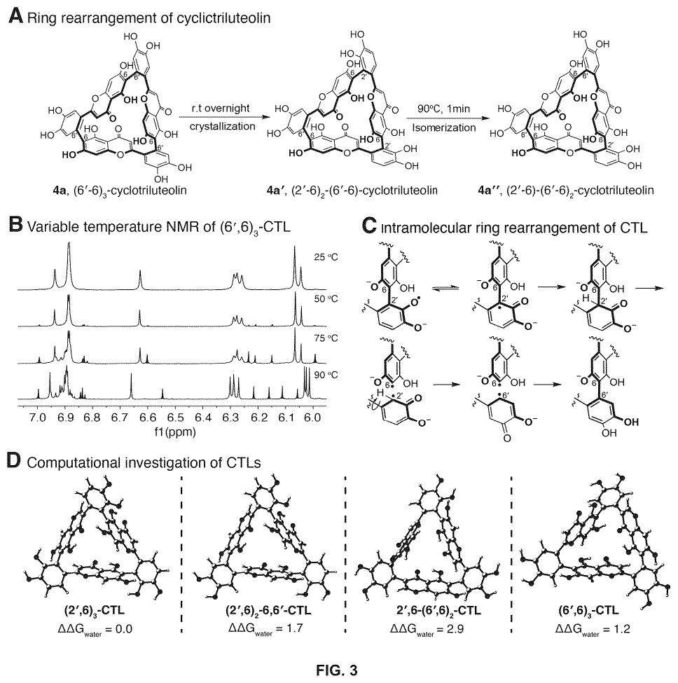

In solution, cyclobartramiatriluteolin exhibited one set of the ¹H and ¹³C NMR spectral peaks for three luteolin units (H. Geiger et al., Phytochemistry 1995, 39, 465-467). Due to the C3 axis in 4a, its ¹H NMR spectrum is in agreement with magnetically equivalent luteolin units C(sp²)-H at 25° C. (FIG. 3A). However, due to the presence of three chiral axis along with the interflavonyl bonds, three rotamers are present with equal intensities of ¹H NMR signals (FIG. 3A). Upon heating the solution to 90° C. in one minute, interflavonyl bond isomerization occurs rapidly to give the new sets of ¹H signals (FIG. 3B). We proposed that isomerization could occur through ortho-semiquinone radical intermediates facilitated in basic conditions or upon heating (FIG. 3B-C). We calculated the Gibbs free energies of four possible isomers of CTL and found that they have relatively similar free energies (FIG. 3D) that were in agreement with the experimental observations.

### Example 5. Homo Cross-Coupling Reaction of B-Catechol Flavones

One of 1a, 1h-k (0.045 mmol) was dissolved in KOH solution (0.1 M, 3 mL) in a 15 mL centrifuge tube. The pH was adjusted to 9.5, 10.0, 10.5, 11.0, 11.5, 12.0, 12.5 or 13.0 with concentrated KOH before the tube was sealed and kept overnight at room temperature without stirring. The resulting solution was then acidified with concentrated HCl (1.0 mL, 10 M) to give a solution with a pH value of 1˜ 2. Then, the resulting solution was extracted with EA (3×50.0 mL), and the organic layers were combined. Removal of the volatiles in vacuo resulted in a crude solid, which was purified over semi-prep HPLC with automatic fraction collection system to give pure 2a, and 2h-k.

Results and Discussion

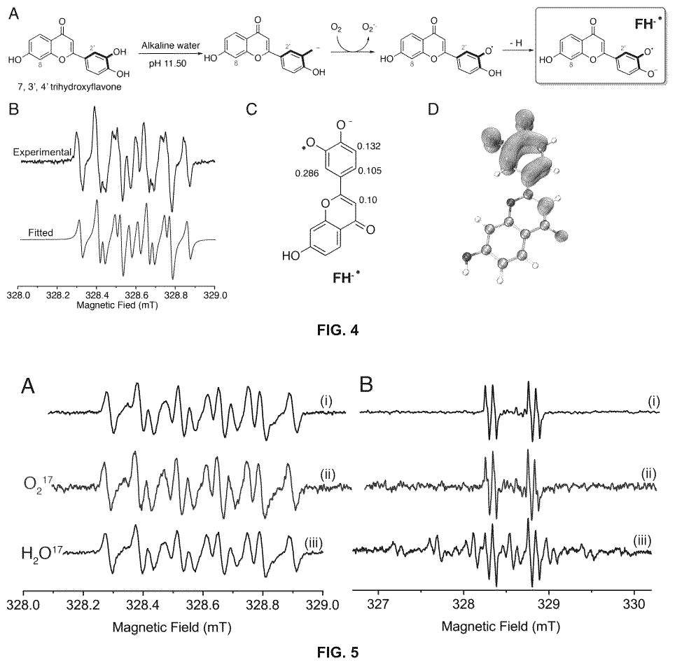

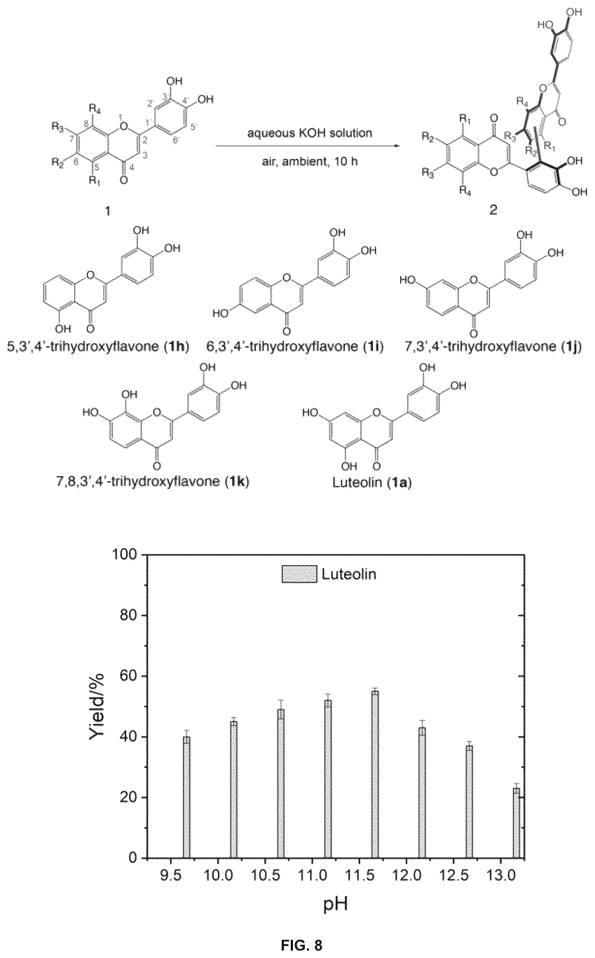

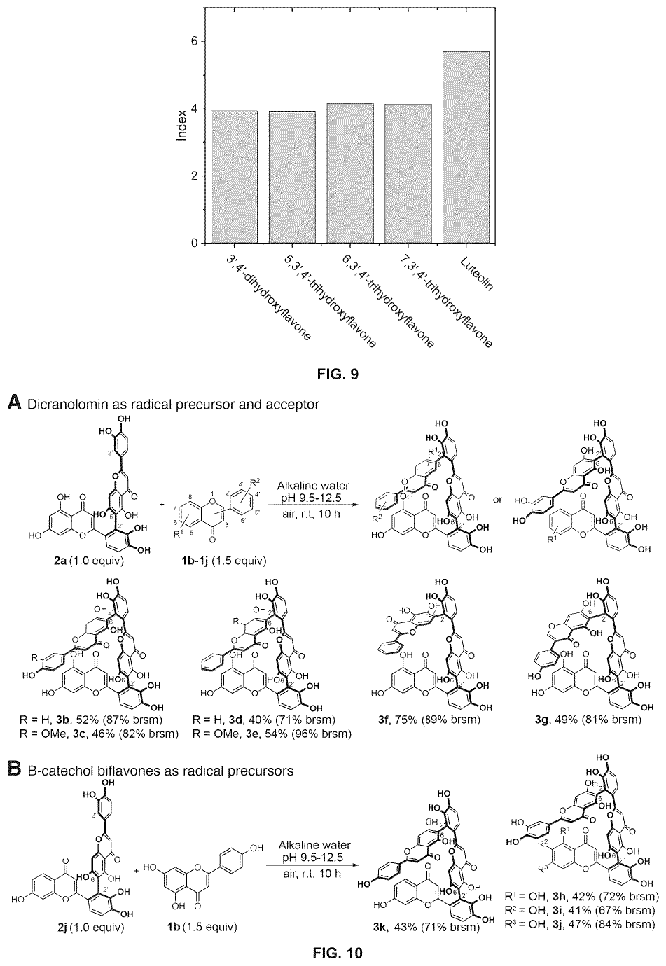

Treating trihydroxyflavones containing catecholic B ring including 3′,4′-dihydroxyflavone, 3′,4′,5-trihydroxyflavone, 3′,4′,6-trihydroxyflavone and 3′,4′,7-trihydroxyflavone in alkaline water resulted in the formation of ortho-semiquinone radicals as detected by EPR spectra (FIG. 4-7). However, there were little coupled reaction products detected (FIG. 8). These observations suggest that these trihydroxyflavones are not sufficiently nucleophilic to accept the semiquinone radicals generated from their oxidation. This is in agreement with the calculation by DFT which found that trihydroxyflavones have much lower nucleophilicity than luteolin (FIG. 9). Hence, luteolin anion is unique among these flavones because of its high nucleophilicity and the ability to form ortho-semiquinone radicals, enabling it to undergo a coupling reaction.

### Example 6. Synthesis of B-Catecholic Flavone-Apigenin Biflavones

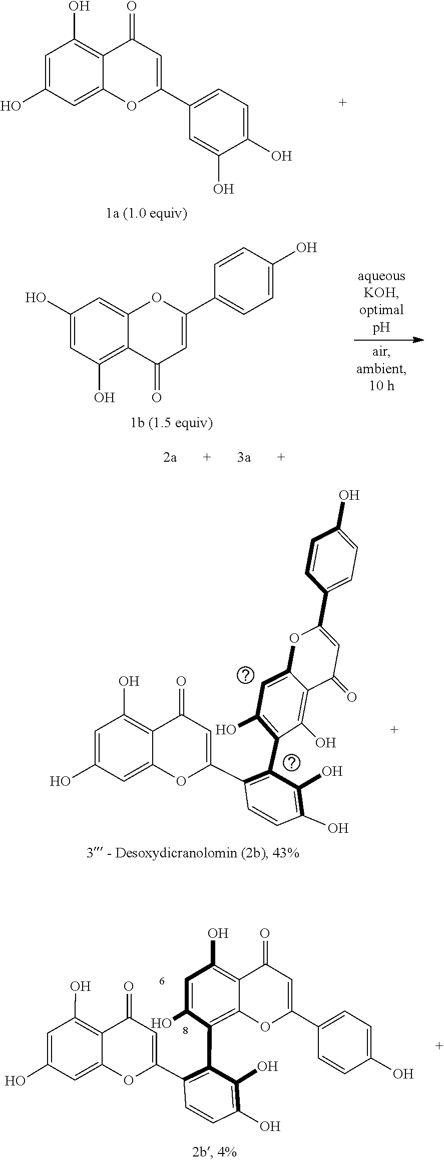

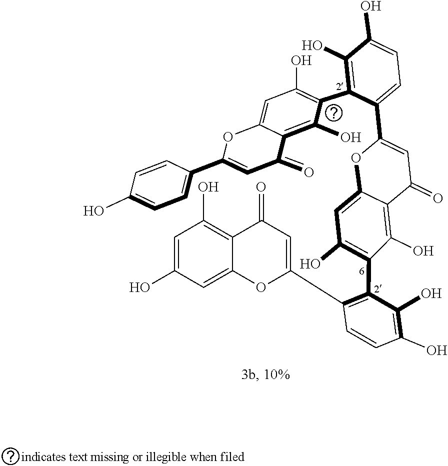

2b was prepared from 1a (0.25 mmol) and Ap (1b, 140 mg, 0.5 mmol) by following the protocol in Example 1 except the product was extracted with 100 mL EA.

43% yield (2b), 4% yield (2b′), 10% yield (3b). Semi-prep HPLC (DI water with 0.1% formic acid/ACN with 0.1% formic acid=71.5:28.5), tᵣ(2b)=31.758 min, tᵣ(2b′)=25.601 min, tᵣ(3b)=35.711 min.

Results and Discussion

When luteolin was mixed with excess Ap (1:1.5 molar ratio), (Lu-(2′-6)-Ap, 2b, was isolated in good yield (47%) together with a trace amount of Lu-(2′-8)-Ap, 2b′, a triflavone (Lu-(2′-6)-Lu-(2′-6)-Ap, 3b), and a trace amount of 2a. The structure of 2b was confirmed by single crystal X-ray diffraction analysis to be desoxydicranolomin (FIG. 10A), a biflavone isolated from Plagiomnium undulatum (C. Rampendahl et al., Phytochemistry 1996, 41, 1621-1624). Taken together, it became apparent that a general rule for oxygen mediated oxidative coupling of two flavones is that one flavone is a radical precursor by forming ortho-semiquinone radical anions, while the other flavone is a good nucleophile under the weakly basic reaction conditions. This rule is valid for luteolin coupling with other flavones, as will be shown in Example 7.

TABLE 8
Condition evaluation of aerobic oxidative luteolin-apigenin cross-coupling^{a}.

| | | | Conversion (%)^{a} | | 2a | 2b + 2b′ | Ratio | 3 | Ratio |
|---|---|---|---|---|---|---|---|---|---|
| Entry | Y | pH | 1a | 1b | Yield (%)^{b} | Yield (%)^{b} | (2b/2b′) | Yield (%)^{b} | (3b/3a) |
| 1 | 1.0 | 13.5 | 97 | 40 | 5 | 1 | 13:1 | 1 | — |
| 2 | 1.0 | 12.5 | 95 | 46 | 28 | 27 | 16:1 | 9 | 3:1 |
| 3 | 1.0 | 11.5 | 91 | 38 | 31 | 24 | 21:1 | 6 | 2:1 |
| 4 | 1.0 | 10.5 | 82 | 30 | 16 | 16 | 14:1 | 3 | 1:1 |
| 5 | 1.0 | 9.5 | 59 | 27 | 15 | 7 | 13:1 | 2 | — |
| 6 | 1.0 | 8.5 | 27 | 17 | 2 | 1 | 18:1 | 1 | — |
| 7 | 1.2 | 12.5 | 89 | 52 | 20 | 37 | 18:1 | 7 | 2:1 |
| 8 | 1.5 | 12.5 | 92 | 65 | 13 | 51 (47^{c}) | 13:1 | 10 | 3:1 |
| 9 | 2.0 | 12.5 | 92 | 66 | 11 | 50 | 10:1 | 12 | 1:1 |
| 10^{d} | 1.5 | 12.5 | 90 | 50 | 11 | 35 | 12:1 | 10 | 1:1 |

^{a}The reaction condition: luteolin 1a (0.045 mmol) and apigenin 1b (Y equiv.) were dissolved in 3 mL KOH solution, incubated in sealed tube 15 mL without stirring.
^{b}Using HPLC to calculate conversion ratio, isocratic elution method (71.5% of mobile phase A: DI water with 0.1% formic acid and 21.5% mobile phase B: ACN with 0.1% formic acid) was applied for products analysis of luteolin-luteolin cross-coupling. Standard curves were built using isolated products.
^{c}Isolated yield.
^{d}pH 12.5 buffer (disodium tetraborate) was applied to take place of KOH solution.

### Example 7. Synthesis of Luteolin-Flavone Biflavones

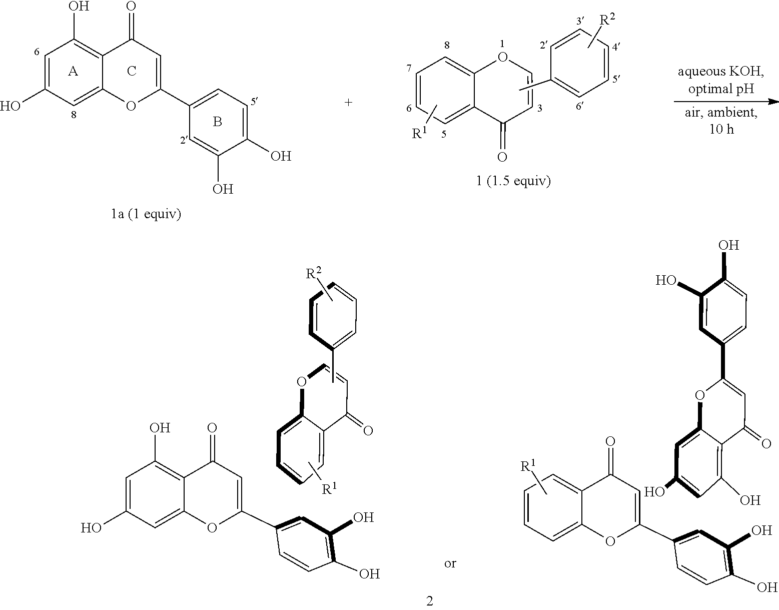

Luteolin-flavone biflavones 2b-2k were prepared from luteolin (72 mg, 0.25 mmol) and a flavone (0.5 mmol) selected from Ap, Dio, Chry, Wo, 5,6-dihydroxyflavone (56), Ge, 5,3′,4′-trihydroxyflavone (534), 6,3′,4′-trihydroxyflavone (634), 7,3′,4′-trihydroxyflavone (734) and 3′,4′-dihydroxyflavone (34), by following the protocol in Example 1.

### 6-(6-(5,7-dihydroxy-4-oxo-4H-chromen-2-yl)-2,3-dihydroxyphenyl)-5,7-dihydroxy-2-(4-hydroxyphenyl)-4H-chromen-4-one (3′-Desoxydicranolomin, 2b)

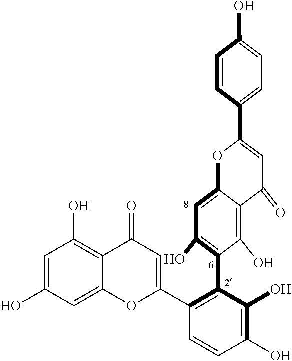

2b was prepared from Ap. 61 mg, 44% yield, brown solid. HPLC (ACN/Water=71.5:28.5, A=300 nm), tᵣ(2b)=30.447 min, concentration of 2b=1.66 mM. ¹H NMR (500 MHz, DMSO-d₆) b 13.15 (s, 1H), 12.80 (s, 1H), 10.34 (s, 5H), 7.95 (d, J=8.4 Hz, 2H), 7.17 (d, J=8.4 Hz, 1H), 7.02-6.86 (m, 3H), 6.77 (s, 1H), 6.54 (s, 1H), 6.09 (d, J=2.1 Hz, 1H), 6.03 (s, 1H), 5.98 (d, J=2.1 Hz, 1H). ¹³C NMR (126 MHz, DMSO) b 182.13, 181.76, 167.11, 164.62, 164.43, 163.85, 161.75, 161.56, 159.22, 157.87, 156.86, 149.00, 145.11, 128.92, 128.92, 124.21, 121.72, 120.61, 120.47, 116.43, 116.43, 114.52, 108.77, 106.60, 103.85, 103.52, 103.26, 99.13, 94.09, 93.81. HRMS (ESI-TOF) calcd for C₃₀H₁₇O₁₁=553.0776, found 553.0782.

Crystals of 2b suitable for X-ray analysis were obtained by slow evaporation from MeOH. A brown Block-like specimen of C₃₀H18011, approximate dimensions 0.061 mm×0.063 mm×0.267 mm, was used for the X-ray crystallographic analysis. The X-ray intensity data were measured. The total exposure time was 19.00 hours. The integration of the data using a triclinic unit cell yielded a total of 18025 reflections to a maximum θ angle of 67.11° (0.84 Å resolution), of which 5625 were independent (average redundancy 3.204, completeness=99.0%, Rᵢₙₜ=6.56%, R_{sig}=6.46%) and 3788 (67.34%) were greater than 2σ(F²). The final cell constants of a=8.4765 Å, b=11.1018 Å, c=18.6315 Å, α=98.306°, R=95.037°, γ=109.024°, volume=1623.0 Å3, are based upon the refinement of the XYZ-centroids of 49 reflections above 20 σ(I) with 8.533°<2θ<40.75°. The ratio of minimum to maximum apparent transmission was 0.883. The structure was solved and refined using the Bruker SHELXTL Software Package, with Z=2 for the formula unit, C₃₀H₁₈O₁₁. The final anisotropic full-matrix least-squares refinement on F² with 491 variables converged at R1=4.71%, for the observed data and wR2=12.43% for all data. The goodness-of-fit was 1.029. The largest peak in the final difference electron density synthesis was 0.253 e⁻/Å³ and the largest hole was −0.240 e⁻/Å³ with an RMS deviation of 0.056 e⁻/Å³. On the basis of the final model, the calculated density was 1.434 g/cm³ and F(000), 720 e⁻. Crystallographic data have been deposited with the Cambridge Cystallograhic Data Centre (CCDC #2044715).

TABLE 9
Sample and crystal data for 2b.

| Identification code | J456 | |
|---|---|---|
| Chemical formula | C₃₀H₁₈O₁₁ | |
| Formula weight | 554.08 | g/mol |
| Temperature | 100(2) | K |
| Wavelength | 1.54184 | Å |
| Crystal size | 0.061 × 0.063 × 0.267 | mm |
| Crystal system | brown Block | |
| Space group | triclinic | |
| Unit cell dimensions | a = 8.4765 Å. α = 98.306° | |
| | b = 11.1018 Å. β = 95.037° | |
| | c = 18.6315 Å. γ = 109.024° | |
| Volume | 1623.0 | Å³ |
| Z | 2 | |
| Density (calculated) | 1.434 | g/cm³ |
| Absorption coefficient | 0.942 | mm⁻¹ |

| | |
|---|---|
| F(000) | 720 |

TABLE 10
Sample and crystal data for 2b.

| | |
|---|---|
| Theta range for data collection | 2.44 to 67.11° |
| Index ranges | −9 <= h <= 10, −13 <= k <= 12, −22 <= l <= 22 |
| Reflections collected | 18025 |
| Independent reflections | 5625 [R(int) = 0.0656] |
| Coverage of independent | 99.00% |
| reflections | |
| Absorption correction | Multi-Scan |
| Structure solution technique | direct methods |
| Structure solution program | XT, VERSION 2014/5 |
| Refinement method | Full-matrix least-squares on F² |
| Refinement program | SHELXL-2016/6 (Sheldrick, 2016) |
| Function minimized | Σ w(Fₒ² − F_{c}²)² |
| Data/restraints/parameters | 5625/0/491 |
| Goodness-of-fit on F2 | 1.029 |
| Final R indices | 3788 data; I > 2σ(I). R1 = 0.0471, wR2 = 0.1063 |
| | all data. R1 = 0.0798, wR2 = 0.1243 |
| Weighting scheme | w = 1/[σ²(Fₒ²) + (0.0578P)² + 0.0599P], where |
| | P = (Fo2 + 2Fc2)/3 |
| Largest diff. peak and hole | 0.253 and −0.240 eÅ⁻³ |
| R.M.S. deviation from mean | 0.056 eÅ⁻³ |

TABLE 11
Atomic coordinates and equivalent isotropic atomic displacement
parameters (Å2) for 2b. U(eq) is defined as one third
of the trace of the orthogonalized Uij tensor.

| | x/a | y/b | z/c | U(eq) |
|---|---|---|---|---|
| O1 | 0.9467(2) | 0.33636(16) | 0.67274(11) | 0.0269(4) |
| O2 | 0.6535(2) | 0.83317(15) | 0.42898(9) | 0.0214(4) |
| O3 | 0.3037(3) | 0.52146(16) | 0.48655(10) | 0.0309(5) |
| O4 | 0.6755(2) | 0.58067(16) | 0.20405(9) | 0.0236(4) |
| O5 | 0.3117(2) | 0.38040(15) | 0.36460(10) | 0.0260(4) |
| O6 | 0.5785(2) | 0.25212(16) | 0.30762(10) | 0.0229(4) |
| O7 | 0.4776(3) | 0.02891(16) | 0.20906(12) | 0.0287(5) |
| O8 | 0.2971(2) | 0.44829(15) | 0.06509(9) | 0.0200(4) |
| O9 | 0.0822(2) | 0.67766(15) | 0.18511(9) | 0.0237(4) |
| O10 | 0.2263(2) | 0.62713(17) | 0.85267(10) | 0.0255(4) |
| O11 | 0.0483(2) | 0.78418(16) | 0.06829(10) | 0.0243(4) |
| C1 | 0.8532(3) | 0.2193(2) | 0.62904(14) | 0.0227(6) |
| C2 | 0.7167(4) | 0.1303(2) | 0.65196(15) | 0.0249(6) |
| C3 | 0.6306(4) | 0.0108(2) | 0.60738(15) | 0.0245(6) |
| C4 | 0.6780(3) | 0.9771(2) | 0.53900(14) | 0.0212(6) |
| C5 | 0.8101(4) | 0.0697(2) | 0.51523(15) | 0.0252(6) |
| C6 | 0.8973(4) | 0.1896(2) | 0.55990(15) | 0.0283(6) |
| C7 | 0.5950(3) | 0.8454(2) | 0.49487(14) | 0.0214(6) |
| C8 | 0.4771(3) | 0.7449(2) | 0.51503(14) | 0.0240(6) |
| C9 | 0.4117(3) | 0.6186(2) | 0.46892(14) | 0.0237(6) |
| C10 | 0.4772(3) | 0.6064(2) | 0.39976(13) | 0.0191(5) |
| C11 | 0.5956(3) | 0.7150(2) | 0.38172(13) | 0.0177(5) |
| C12 | 0.6637(3) | 0.7106(2) | 0.31664(14) | 0.0203(5) |
| C13 | 0.6111(3) | 0.5915(2) | 0.26870(14) | 0.0194(5) |
| C14 | 0.4916(3) | 0.4780(2) | 0.28277(13) | 0.0186(5) |
| C15 | 0.4266(3) | 0.4869(2) | 0.34909(14) | 0.0190(5) |
| C16 | 0.4335(3) | 0.3524(2) | 0.22842(13) | 0.0179(5) |
| C17 | 0.4809(3) | 0.2480(2) | 0.24422(14) | 0.0194(5) |
| C18 | 0.4305(3) | 0.1300(2) | 0.19377(15) | 0.0216(6) |
| C19 | 0.3323(3) | 0.1155(2) | 0.12715(14) | 0.0234(6) |
| C20 | 0.2887(3) | 0.2193(2) | 0.10934(15) | 0.0232(6) |
| C21 | 0.3372(3) | 0.3372(2) | 0.15929(14) | 0.0190(5) |
| C22 | 0.2800(3) | 0.4401(2) | 0.13727(13) | 0.0183(5) |
| C23 | 0.2115(3) | 0.5163(2) | 0.17812(14) | 0.0199(5) |
| C24 | 0.1506(3) | 0.6080(2) | 0.14789(14) | 0.0191(5) |
| C25 | 0.1701(3) | 0.6155(2) | 0.07159(13) | 0.0187(5) |
| C26 | 0.2421(3) | 0.5343(2) | 0.03218(14) | 0.0186(5) |
| C27 | 0.2663(3) | 0.5357(2) | 0.95974(14) | 0.0195(5) |
| C28 | 0.2139(3) | 0.6218(2) | 0.92416(14) | 0.0204(5) |
| C29 | 0.1411(3) | 0.7054(2) | 0.96083(14) | 0.0215(6) |
| C30 | 0.1195(3) | 0.7031(2) | 0.03384(14) | 0.0196(5) |
| O1S | 0.7523(3) | 0.03701(18) | 0.29848(11) | 0.0341(5) |
| C1S | 0.9389(4) | 0.2276(3) | 0.26425(17) | 0.0338(7) |
| C2S | 0.8981(4) | 0.1064(3) | 0.29708(16) | 0.0308(7) |
| C3S | 0.0436(4) | 0.0718(3) | 0.3274(2) | 0.0477(9) |
| O2S | 0.8363(3) | 0.81958(16) | 0.17516(10) | 0.0294(5) |
| C4S | 0.8367(4) | 0.9840(3) | 0.10535(18) | 0.0359(7) |
| C5S | 0.7560(4) | 0.8598(2) | 0.13223(16) | 0.0275(6) |
| C6S | 0.5743(4) | 0.7894(3) | 0.10549(19) | 0.0431(8) |
| O3S | 0.8282(3) | 0.45074(18) | 0.38282(11) | 0.0289(5) |

### 8-(6-(5,7-dihydroxy-4-oxo-4H-chromen-2-yl)-2,3-dihydroxyphenyl)-5,7-dihydroxy-2-(4-hydroxyphenyl)-4H-chromen-4-one (Lu-Ap (2-8), 2b′)

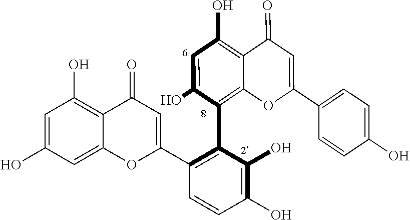

2b′ was prepared from Ap. 4 mg, 3% yield, brown solid. HPLC (ACN/Water=71.5:28.5, λ=300 nm), tᵣ(2b′)=23.852 min, concentration of 2b′=8.3 mM. ¹H NMR (500 MHz, DMSO-d₆) δ 13.01 (s, 1H), 12.75 (s, 1H), 10.76 (s, 3H), 10.33 (s, 2H), 7.53 (d, J=9.1 Hz, 2H), 7.33-7.22 (d, J=8.2 Hz, 1H), 7.03 (d, J=8.2 Hz, 1H), 6.81 (d, J=9.1 Hz, 2H), 6.76 (s, 1H), 6.28 (d, J=1.4 Hz, 1H), 6.08 (d, J=2.1 Hz, 1H), 6.04 (d, J=1.4 Hz, 1H), 5.76 (d, J=2.1 Hz, 1H). ¹³C NMR (126 MHz, DMSO) δ 207.07, 182.45, 181.71, 167.05, 164.43, 164.02, 162.11, 161.70, 161.51, 160.89, 157.71, 154.72, 148.99, 144.94, 128.59, 124.43, 121.73, 121.12, 119.26, 116.27, 115.08, 106.92, 104.00, 103.76, 103.58, 102.96, 99.17, 98.85, 93.63. HRMS (ESI-TOF) calcd for C₃₀H₁₇O₁₁=553.0776, found 553.0782.

### 6-(6-(5,7-dihydroxy-4-oxo-4H-chromen-2-yl)-2,3-dihydroxyphenyl)-5,7-dihydroxy-2-(3-hydroxy-4-methoxyphenyl)-4H-chromen-4-one (Lu-Dio (2′-6), 2c)

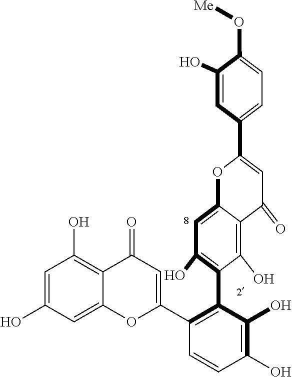

2c was prepared from Dio. 95 mg, 65% yield, brown solid. HPLC (ACN/Water=64:36, λ=300 nm), tᵣ(2c)=11.018 min, concentration of 2c=0.81 mM. ¹H NMR (500 MHz, DMSO-d₆) δ 13.14 (s, 1H), 12.82 (s, 1H), 10.52 (s, 4H), 9.51 (s, 1H), 7.56 (dd, J=8.7, 2.3 Hz, 1H), 7.45 (d, J=2.4 Hz, 1H), 7.17 (d, J=8.3 Hz, 1H), 7.10 (d, J=8.7 Hz, 1H), 6.92 (d, J=8.3 Hz, 1H), 6.73 (s, 1H), 6.51 (s, 1H), 6.09 (d, J=2.1 Hz, 1H), 6.04 (s, 1H), 5.99 (d, J=2.1 Hz, 1H), 3.88 (s, 3H). ¹³C NMR (126 MHz, DMSO) δ 182.00, 181.77, 167.22, 164.43, 163.54, 161.75, 159.21, 157.87, 156.90, 151.52, 149.11, 147.27, 145.32, 124.21, 123.59, 120.57, 119.12, 114.42, 113.35, 112.68, 109.01, 106.52, 103.88, 103.85, 103.39, 99.12, 94.19, 93.81, 56.23. HRMS (ESI-TOF) calcd for C₃₁H₁₉O₁₂=583.0882, found 583.0898.

### 6-(6-(5,7-dihydroxy-4-oxo-4H-chromen-2-yl)-2,3-dihydroxyphenyl)-5,7-dihydroxy-2-phenyl-4H-chromen-4-one (Lu-Chry (2-6), 2d)

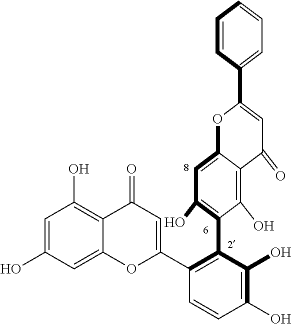

2d was prepared from Chry. 62 mg, 46% yield, brown solid. HPLC (ACN/Water=60:40, λ=300 nm), tᵣ(2d)=12.096 min, concentration of 2d=2.40 mM. ¹H NMR (400 MHz, DMSO-d₆) δ 13.01 (s, 1H), 12.79 (s, 1H), 11.14-10.09 (m, 4H), δ 8.16-8.05 (m, 2H), 7.68-7.53 (m, 3H), 7.20 (d, J=8.4 Hz, 1H), 7.00 (s, 1H), 6.95 (d, J=8.4 Hz, 1H), 6.62 (s, 1H), 6.09 (d, J=2.1 Hz, 1H), 6.07 (s, 1H), 5.98 (d, J=2.1 Hz, 1H). ¹³C NMR (126 MHz, DMSO) δ 182.38, 181.76, 166.90, 164.47, 163.46, 162.78, 161.77, 159.23, 157.85, 156.96, 148.84, 144.79, 132.47, 131.20, 129.63, 129.63, 126.91, 126.91, 124.19, 120.75, 120.13, 114.79, 108.75, 106.74, 105.68, 104.13, 103.86, 99.16, 93.97, 93.80. HRMS (ESI-TOF) calcd for C₃₀H₁₇O₁₀=537.0827, found 537.0824.

### 6-(6-(5,7-dihydroxy-4-oxo-4H-chromen-2-yl)-2,3-dihydroxyphenyl)-5,7-dihydroxy-8-methoxy-2-phenyl-4H-chromen-4-one (Lu-Wo (2-6), 2e)

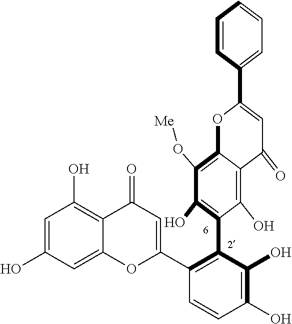

2e was prepared from Wo. 121 mg, 85% yield, brown solid. HPLC (ACN/Water=60:40, λ=300 nm), tᵣ(2e)=25.651 min, concentration of 2e=1.95 mM. ¹H NMR (500 MHz, DMSO-d₆) 12.80 (s, 1H), 12.73 (s, 1H), 10.73 (s, 1H), 10.29 (s, 1H), 10.18 (s, 1H), 8.53 (s, 1H), 8.18-8.05 (m, 2H), 7.68-7.55 (m, 3H), 7.22 (d, J=8.4 Hz, 1H), 7.06 (s, 1H), 6.97 (d, J=8.4 Hz, 1H), 6.09 (d, J=2.1 Hz, 1H), 6.08 (s, 1H), 5.91 (d, J=2.1 Hz, 1H), 3.82 (s, 3H). ¹³C NMR (126 MHz, DMSO) δ 182.56, 181.77, 166.86, 164.44, 163.29, 161.76, 157.78, 155.48, 154.52, 149.05, 148.86, 144.75, 132.57, 131.28, 129.77, 127.91, 126.76, 124.07, 120.78, 119.76, 114.90, 108.89, 106.79, 105.69, 103.85, 103.82, 99.14, 93.75, 61.95. HRMS (ESI-TOF) calcd for C₃₁H₁₉O₁₁=567.0933, found 567.0921.

Crystals of compound 2e suitable for X-ray analysis were obtained by slow evaporation from MeOH and DMSO. A specimen of C₃₁H₂₀O₁₁, approximate dimensions 0.049 mm×0.122 mm×0.132 mm, was used for the X-ray crystallographic analysis. The X-ray intensity data were measured (λ=1.54178 Å). The total exposure time was 17.13 hours. The integration of the data using a triclinic unit cell yielded a total of 8055 reflections to a maximum θ angle of 67.03° (0.84 Å resolution), of which 8055 were independent (average redundancy 1.000, completeness=97.6%, R_{sig}=10.51%) and 4577(56.82%) were greater than 2σ(F²). The final cell constants of a=8.6271(6) Å, b=10.2328(6) Å, c=17.4619(12) Å, α=102.701(4°), β=102.737(4°), γ=90.020(4°), volume=1464.87(17) Å³, are based upon the refinement of the XYZ-centroids of 4286 reflections above 20 σ(I) with 8.870°<2θ<133.1°. The ratio of minimum to maximum apparent transmission was 0.637. The calculated minimum and maximum transmission coefficients (based on crystal size) were 0.4799 and 0.7528. The structure was solved and refined using the Bruker SHELXTL Software Package, using the space group P−1, with Z=2 for the formula unit, C₃₁H₂₀O₁₁. The final anisotropic full-matrix least-squares refinement on F² with 455 variables converged at R1=9.67%, for the observed data and wR2=32.15% for all data. The goodness-of-fit was 1.065. The largest peak in the final difference electron density synthesis was 0.495 e⁻/Å³ and the largest hole was −0.499 e⁻/Å³ with an RMS deviation of 0.104 e⁻/Å³. On the basis of the final model, the calculated density was 1.475 g/cm³ and F(000), 676 e⁻. Crystallographic data have been deposited with the Cambridge Crystallograhic Data Centre (CCDC #2044716).

TABLE 12
Sample and crystal data for 2e.

| Identification code | J357 | |
|---|---|---|
| Chemical formula | C₃₁H₂₀O₁₁ | |
| Formula weight | 650.58 | g/mol |
| Temperature | 100(2) | K |
| Wavelength | 1.54178 | Å |
| Crystal size | 0.049 × 0.122 × 0.132 | mm |
| Crystal system | triclinic | |
| Space group | P −1 | |
| Unit cell dimensions | a = 8.6271(6) Å. α = 102.701(4)° | |
| | b = 10.2328(6) Å. β = 102.737(4)° | |
| | c = 17.4619(12) Å. γ = 90.020(4)° | |
| Volume | 1464.87(17) | Å³ |
| Z | 2 | |
| Density (calculated) | 1.475 | g/cm³ |
| Absorption coefficient | 0.936 | mm⁻¹ |

| | |
|---|---|
| F(000) | 676 |

TABLE 13
Data collection and structure refinement for 2e.

| | |
|---|---|
| Theta range for data collection | 2.66 to 67.03° |
| Index ranges | −10 <= h <= 10, −11 <= k <= 12, −20 <= l <= 20 |
| Reflections collected | 8055 |
| Coverage of independent | 97.60% |
| reflections | |
| Absorption correction | Multi-Scan |
| Max. and min. transmission | 0.7528 and 0.4799 |
| Structure solution technique | direct methods |
| Structure solution program | SHELXS-97 (Sheldrick 2008) |
| Refinement method | Full-matrix least-squares on F² |
| Refinement program | SHELXL-2017/1 (Sheldrick, 2017) |
| Function minimized | Σ w(Fₒ² − F_{c}²)² |
| Data/restraints/parameters | 8055/6/455 |
| Goodness-of-fit on F2 | 1.065 |
| Final R indices | 4577 data; I > 2σ(I). R1 = 0.0967, wR2 = 0.2670 |
| | all data. R1 = 0.1466, wR2 = 0.3215 |
| Weighting scheme | w = 1/[σ²(Fₒ²) + (0.1943P)²], where P = (Fo2 + 2Fc2)/3 |
| Largest diff. peak and hole | 0.495 and −0.499 eÅ⁻³ |
| R.M.S. deviation from mean | 0.104 eÅ⁻³ |

TABLE 14
Atomic coordinates and equivalent isotropic atomic displacement
parameters (Å2) for 2e. U(eq) is defined as one third
of the trace of the orthogonalized Uij tensor.

| | x/a | y/b | z/c | U(eq) |
|---|---|---|---|---|
| O1 | 0.5470(5) | 0.1328(4) | 0.8847(2) | 0.0397(10) |
| O2 | 0.2969(5) | 0.2518(4) | 0.4914(2) | 0.0396(10) |
| O3 | 0.9576(5) | 0.3840(4) | 0.0831(3) | 0.0467(11) |
| O4 | 0.5791(5) | 0.6137(4) | 0.9259(3) | 0.0495(11) |
| O5 | 0.3462(5) | 0.4678(4) | 0.8222(3) | 0.0453(11) |
| O6 | 0.1452(5) | 0.5950(4) | 0.7217(3) | 0.0442(10) |
| O7 | 0.1277(5) | 0.7429(4) | 0.6147(2) | 0.0417(10) |
| O8 | 0.5584(5) | 0.9941(4) | 0.6773(3) | 0.0422(10) |
| O9 | 0.0045(5) | 0.0345(4) | 0.6309(3) | 0.0410(10) |
| O10 | 0.0134(4) | 0.2033(4) | 0.5284(2) | 0.0394(10) |
| O11 | 0.7115(5) | 0.1071(4) | 0.5959(3) | 0.0445(10) |
| C1 | 0.6189(7) | 0.2571(6) | 0.9230(3) | 0.0393(13) |
| C2 | 0.7542(7) | 0.2598(6) | 0.9823(3) | 0.0389(13) |
| C3 | 0.8285(7) | 0.3834(7) | 0.0226(4) | 0.0433(14) |
| C4 | 0.7718(7) | 0.5030(7) | 0.0030(4) | 0.0436(14) |
| C5 | 0.6354(7) | 0.4973(6) | 0.9430(4) | 0.0432(14) |
| C6 | 0.5552(7) | 0.3740(6) | 0.9011(4) | 0.0400(14) |
| C7 | 0.4091(7) | 0.3633(7) | 0.8408(4) | 0.0430(14) |
| C8 | 0.3408(7) | 0.2314(6) | 0.8062(4) | 0.0408(14) |
| C9 | 0.4083(7) | 0.1222(6) | 0.8277(4) | 0.0411(14) |
| C10 | 0.3445(7) | 0.9833(6) | 0.7976(4) | 0.0368(13) |
| C11 | 0.3516(7) | 0.9033(6) | 0.8534(4) | 0.0428(14) |
| C12 | 0.2840(7) | 0.7746(6) | 0.8280(4) | 0.0437(14) |
| C13 | 0.2097(7) | 0.7214(6) | 0.7494(4) | 0.0385(13) |
| C14 | 0.1999(6) | 0.8023(6) | 0.6926(4) | 0.0376(13) |
| C15 | 0.2724(7) | 0.9314(6) | 0.7153(4) | 0.0376(13) |
| C16 | 0.2804(7) | 0.0097(6) | 0.6530(3) | 0.0375(13) |
| C17 | 0.1431(7) | 0.0627(6) | 0.6140(3) | 0.0363(13) |
| C18 | 0.1503(7) | 0.1472(6) | 0.5612(3) | 0.0377(13) |
| C19 | 0.2958(7) | 0.1732(6) | 0.5453(3) | 0.0381(13) |
| C20 | 0.4358(7) | 0.1211(6) | 0.5822(4) | 0.0372(13) |
| C21 | 0.4253(7) | 0.0401(6) | 0.6387(4) | 0.0391(13) |
| C22 | 0.5833(7) | 0.1502(6) | 0.5629(4) | 0.0394(13) |
| C23 | 0.5730(7) | 0.2297(6) | 0.5036(4) | 0.0397(13) |
| C24 | 0.4350(7) | 0.2787(6) | 0.4716(4) | 0.0375(13) |
| C25 | 0.4122(7) | 0.3626(6) | 0.4113(3) | 0.0386(13) |
| C26 | 0.5437(7) | 0.4211(6) | 0.3950(4) | 0.0416(14) |
| C27 | 0.5228(8) | 0.5009(6) | 0.3391(4) | 0.0446(14) |
| C28 | 0.3726(8) | 0.5237(6) | 0.2992(4) | 0.0445(14) |
| C29 | 0.2416(7) | 0.4650(7) | 0.3144(4) | 0.0449(15) |
| C30 | 0.2589(7) | 0.3852(6) | 0.3710(4) | 0.0430(14) |
| C31 | 0.0048(8) | 0.3408(6) | 0.5716(4) | 0.0464(15) |
| C32 | 0.0672(8) | 0.7611(8) | 0.1792(4) | 0.0492(16) |
| C33 | 0.0771(9) | 0.9054(7) | 0.1826(4) | 0.0531(17) |
| C34 | 0.8008(10) | 0.8628(8) | 0.9359(6) | 0.067(2) |
| C35 | 0.7708(9) | 0.8023(8) | 0.8511(5) | 0.0619(19) |

### 7-(6-(5,7-dihydroxy-4-oxo-4H-chromen-2-yl)-2,3-dihydroxyphenyl)-5,6-dihydroxy-2-phenyl-4H-chromen-4-one (Lu-56 (2′-7), 2f)

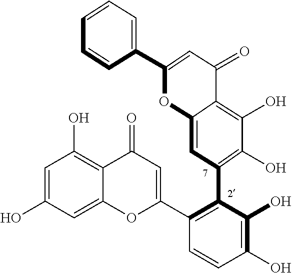

2f was prepared from 5,6-dihydroxyflavone. 77 mg, 57% yield, brown solid. HPLC (ACN/Water=64:36, λ=300 nm), tᵣ(2f)=19.290 min, concentration of 2f=1.95 mM. ¹H NMR (500 MHz, DMSO-d₆) δ 13.25 (s, 1H), 12.84 (s, 1H), 11.04 (s, 1H), 9.95 (s, 1H), 9.58 (s, 1H), 9.12 (s, 1H), 7.68-7.57 (m, 2H), 7.52-7.44 (m, 1H), 7.44-7.29 (m, 3H), 7.04 (s, 1H), 6.96-6.84 (m, 2H), 6.71 (s, 1H), 6.66 (d, J=8.9 Hz, 1H), 6.50 (s, 1H). ¹³C NMR (126 MHz, DMSO) δ 184.38, 182.45, 164.32, 164.10, 162.30, 161.40, 154.81, 150.11, 146.87, 146.39, 146.10, 141.03, 132.58, 131.18, 129.52, 126.42, 126.37, 121.82, 118.75, 116.04, 113.72, 111.16, 110.67, 104.86, 104.21, 103.06, 102.12, 99.19. HRMS (ESI-TOF) calcd for C₃₀H₁₇O₁₀=537.0827, found 537.0837.

### 8-(6-(5,7-dihydroxy-4-oxo-4H-chromen-2-yl)-2,3-dihydroxyphenyl)-5,6-dihydroxy-2-phenyl-4H-chromen-4-one (Lu-56 (2-8), 2f)

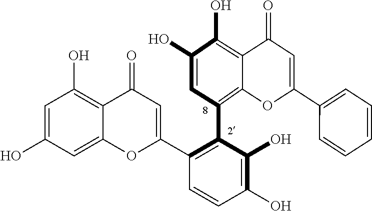

2f was prepared from 5,6-dihydroxyflavone. 8 mg, 6% yield, brown solid. HPLC (ACN/Water=64:36, λ=300 nm), tᵣ(2f)=25.948 min, concentration of 2f=2.50 mM. ¹H NMR (500 MHz, DMSO-d₆) δ 13.52 (s, 1H), 12.73 (s, 1H), 9.51 (s, 4H), 7.82 (d, J=8.2 Hz, 2H), 7.53 (d, J=7.2 Hz, 1H), 7.48 (q, J=8.2, 7.2 Hz, 4H), 7.25 (s, 1H), 7.05 (s, 1H), 6.93 (s, 1H), 6.77 (s, 1H), 6.73 (s, 1H). ¹³C NMR (126 MHz, DMSO) δ 184.37, 182.28, 164.42, 164.23, 162.54, 159.59, 157.07, 150.30, 146.54, 146.27, 140.78, 132.59, 131.42, 129.66, 126.71, 126.36, 121.89, 119.59, 116.56, 113.87, 111.51, 111.05, 106.90, 104.85, 103.99, 103.38, 93.96. HRMS (ESI-TOF) calcd for C₃₀H₁₇O₁₀=537.0827, found 537.0830.

### 6-(6-(5,7-dihydroxy-4-oxo-4H-chromen-2-yl)-2,3-dihydroxyphenyl)-5,7-dihydroxy-3-(4-hydroxyphenyl)-4H-chromen-4-one (Lu-Ge (2′-6), 2q)

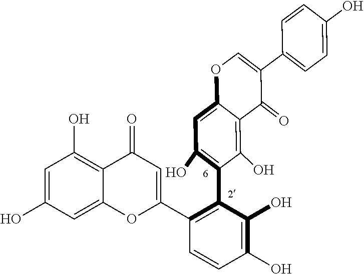

2g was prepared from Ge. 39 mg, 28% yield, brown solid. HPLC (ACNA/Water=71.5:28.5, A=300 nm), tᵣ(2g)=28.867 min, concentration of 2g=1.13 mM. ¹H NMR (500 MHz, DMSO-d₆) δ 13.13 (s, 1H), 12.78 (s, 1H), 10.73 (s, 3H), 10.15 (s, 1H), 9.58 (s, 2H), 8.34 (s, 1H), 7.42-7.34 (m, 2H), 7.18 (d, J=8.4 Hz, 1H), 6.94 (d, J=8.4 Hz, 1H), 6.86-6.75 (m, 2H), 6.48 (s, 1H), 6.10 (d, J=2.1 Hz, 1H), 6.03 (s, 1H), 5.99 (d, J=2.1 Hz, 1H). ¹³C NMR (126 MHz, DMSO) δ 181.75, 180.73, 166.83, 164.48, 162.64, 161.77, 159.68, 157.85, 157.85, 157.07, 154.28, 148.81, 144.75, 130.67, 130.67, 124.19, 122.75, 121.74, 120.74, 120.19, 115.50, 115.50, 114.77, 108.59, 106.75, 104.62, 103.87, 99.16, 93.82, 93.47. HRMS (ESI-TOF) calcd for C₃₀H₁₇O₁₁=553.0776, found 553.0781.

### 8-(6-(5,7-dihydroxy-4-oxo-4H-chromen-2-yl)-2,3-dihydroxyphenyl)-5,7-dihydroxy-3-(4-hydroxyphenyl)-4H-chromen-4-one (Lu-Ge (2′-8), 2′)

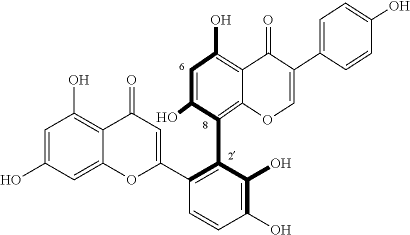

2g′ was prepared from Ge. 19 mg, 14% yield, brown solid. HPLC (ACN/Water=71.5:28.5, A=300 nm), tᵣ(2g′)=29.696 min, concentration of 2g′=0.51 mM. ¹H NMR (500 MHz, DMSO-d₆) δ 13.08 (s, 1H), 12.80 (s, 1H), 10.70 (s, 3H), 9.59 (s, 2H), 8.30 (s, 1H), 7.38-7.32 (m, 2H), 7.21 (d, J=8.4 Hz, 1H), 6.97 (d, J=8.4 Hz, 1H), 6.84-6.77 (m, 2H), 6.30 (s, 1H), 6.11 (d, J=2.1 Hz, 1H), 6.08 (s, 1H), 5.85 (d, J=2.1 Hz, 1H). ¹³C NMR (126 MHz, DMSO) δ 181.78, 180.87, 166.98, 164.49, 162.41, 161.77, 161.45, 157.84, 157.77, 155.50, 154.50, 149.08, 145.12, 130.57, 130.57, 124.42, 122.46, 121.66, 121.10, 119.26, 115.51, 115.51, 115.00, 106.92, 104.78, 103.83, 103.48, 99.18, 99.12, 93.74. HRMS (ESI-TOF) calcd for C₃₀H₁₇O₁₁=553.0776, found 553.0776.

### 6-(2,3-dihydroxy-6-(5-hydroxy-4-oxo-4H-chromen-2-yl)phenyl)-2-(3,4-dihydroxyphenyl)-5,7-dihydroxy-4H-chromen-4-one (534-Lu (2-6), 2h)

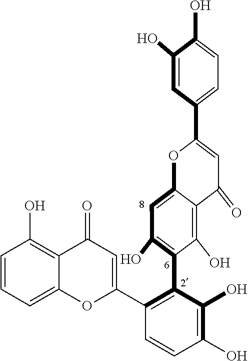

2h was prepared from 5,3′,4′-trihydroxyflavone. 60 mg, 43% yield, brown solid. HPLC (ACN/Water=64:36, λ=300 nm), tᵣ(2h)=14.195 min, concentration of 2h=4.29 mM. ¹H NMR (500 MHz, DMSO-d₆) δ 13.16 (s, 1H), 12.64 (s, 1H), 9.76 (s, 5H), 7.53 (t, J=8.2 Hz, 1H), 7.48-7.40 (m, 2H), 7.25 (d, J=8.4 Hz, 1H), 6.97 (d, J=8.4 Hz, 1H), 6.91 (d, J=8.2 Hz, 1H), 6.74-6.67 (m, 2H), 6.65 (d, J=8.4, 1H), 6.56 (s, 1H), 6.20 (s, 1H). ¹³C NMR (126 MHz, DMSO) δ 182.95, 182.16, 167.91, 164.19, 162.43, 160.24, 159.28, 156.83, 156.36, 150.19, 149.14, 146.22, 144.86, 136.16, 124.01, 121.94, 121.01, 120.36, 119.49, 116.54, 114.84, 113.78, 111.12, 110.09, 108.40, 107.34, 107.18, 103.87, 103.33, 93.71. HRMS (ESI-TOF) calcd for C₃₀H₁₉O₁₁=553.0776, found 553.0735.

### 6-(2,3-dihydroxy-6-(6-hydroxy-4-oxo-4H-chromen-2-yl)phenyl)-5,7-dihydroxy-2-(4-hydroxyphenyl)-4H-chromen-4-one (634-Lu (2-6), 2i)

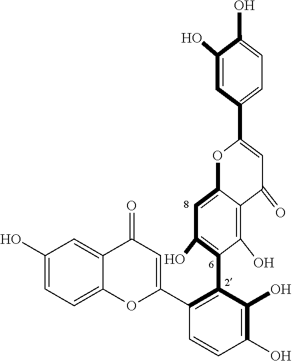

2h was prepared from 6,3′,4′-trihydroxyflavone. 51 mg, 37% yield, brown solid. HPLC (ACN/Water=72.5:27.5, λ=300 nm), tᵣ(2i)=9.532 min, concentration of 2i=6.99 mM. ¹H NMR (500 MHz, DMSO-d₆) δ 13.12 (s, 1H), 10.70 (s, 1H), 10.06 (s, 1H), 9.92 (s, 1H), 9.45 (s, 1H), 8.43 (s, 1H), 8.14 (s, 1H), 7.50-7.39 (m, 2H), 7.26-7.08 (m, 4H), 6.96 (d, J=8.4 Hz, 1H), 6.91 (d, J=8.2 Hz, 1H), 6.69 (s, 1H), 6.54 (s, 1H), 6.05 (s, 1H). ¹³C NMR (126 MHz, DMSO) δ 182.16, 176.92, 165.67, 164.16, 163.51, 162.40, 159.31, 156.74, 155.05, 150.15, 149.90, 148.35, 146.21, 144.69, 125.02, 124.29, 123.24, 121.97, 120.42, 120.15, 119.55, 116.53, 114.77, 113.79, 108.57, 108.07, 107.81, 103.87, 103.33, 93.62. HRMS (ESI-TOF) calcd for C₃₀H₁₉O₁₁=553.0776, found 553.0769.

### 6-(2,3-dihydroxy-6-(7-hydroxy-4-oxo-4H-chromen-2-yl)phenyl)-2-(3,4-dihydroxyphenyl)-5,7-dihydroxy-4H-chromen-4-one (734-Lu (2-6), 2j)

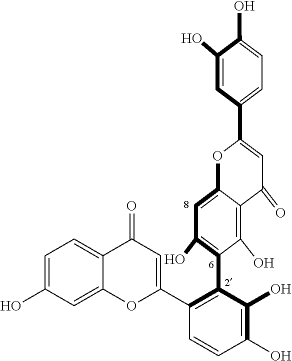

2h was prepared from 7,3′,4′-trihydroxyflavone. 59 mg, 43% yield, brown solid. HPLC (ACN/Water=72.5:27.5, λ=300 nm), tᵣ(2j)=9.790 min, concentration of 2j=9.25 mM. ¹H NMR (500 MHz, DMSO-d₆) δ 13.14 (s, 1H), 10.69 (d, J=8.2 Hz, 2H), 10.06 (s, 1H), 9.94 (s, 1H), 9.46 (s, 1H), 8.42 (s, 1H), 7.76 (d, J=8.6 Hz, 1H), 7.45 (d, J=8.6 Hz, 2H), 7.18 (d, J=8.3 Hz, 1H), 6.96 (d, J=8.3 Hz, 1H), 6.92 (d, J=8.1 Hz, 1H), 6.82 (dd, J=8.1, 2.1 Hz, 1H), 6.70 (s, 1H), 6.55 (s, 1H), 6.50 (d, J=2.1 Hz, 1H), 6.03 (s, 1H). ¹³C NMR (126 MHz, DMSO) δ 207.17, 182.21, 176.60, 165.55, 164.17, 162.85, 162.35, 159.29, 157.97, 156.76, 150.15, 148.31, 146.22, 144.67, 126.83, 124.93, 122.02, 120.47, 120.11, 119.49, 116.56, 116.22, 115.22, 114.80, 113.78, 108.63, 108.59, 103.90, 103.37, 102.36, 93.64. HRMS (ESI-TOF) calcd for C₃₀H₁₉O₁₁=553.0776, found 555.0732.

### 6-(2,3-dihydroxy-6-(4-oxo-4H-chromen-2-yl)phenyl)-2-(3,4-dihydroxyphenyl)-5,7-dihydroxy-4H-chromen-4-one (34-Lu (2-6), 34: 3′,4′-dihydroxyflavone, 2k)

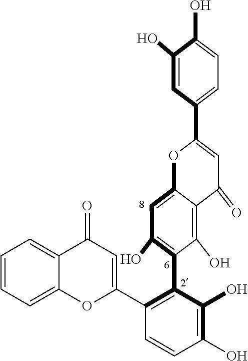

2k was prepared from 3′,4′-dihydroxyflavone. 67 mg, 50% yield, brown solid. HPLC (ACN/Water=71.5:28.5, λ=300 nm), tᵣ(2k)=18.289 min, concentration of 2k=3.25 mM. ¹H NMR (400 MHz, DMSO-d₆) δ 13.14 (s, 1H), 9.69 (s, 5H), 7.92 (d, J=7.9 Hz, 1H), 7.68 (d, J=7.9 Hz, 1H), 7.47-7.35 (m, 3H), 7.25 (dd, J=16.9, 8.3 Hz, 2H), 6.97 (d, J=8.3 Hz, 1H), 6.90 (d, J=8.3 Hz, 1H), 6.68 (s, 1H), 6.54 (s, 1H), 6.13 (s, 1H). ¹³C NMR (126 MHz, DMSO) δ 182.14, 177.05, 166.20, 164.14, 162.64, 159.31, 156.78, 156.17, 150.17, 148.62, 146.22, 144.82, 134.46, 125.66, 125.15, 124.73, 123.41, 121.95, 120.57, 120.28, 119.48, 118.23, 116.53, 114.75, 113.77, 108.98, 108.61, 103.80, 103.30, 93.71. HRMS (ESI-TOF) calcd for C₃₀H₁₇O₁₀=537.0827, found 537.0818.

### 6-(2,3-dihydroxy-6-(5-hydroxy-4-oxo-4H-chromen-2-yl)phenyl)-5,7-dihydroxy-2-(4-hydroxyphenyl)-4H-chromen-4-one (534-Ap (2-6), 21)

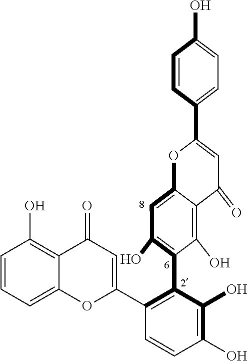

21 was prepared from Ap. 73 mg, 54% yield, brown solid. HPLC (ACN/Water=64:36, λ=300 nm), tᵣ(21)=21.513 min, concentration of 21=15.80 mM. ¹H NMR (500 MHz, DMSO-d₆) δ 13.19 (s, 1H), 12.63 (s, 1H), 10.81 (s, 1H), 10.40 (s, 1H), 10.26 (s, 1H), 8.55 (s, 1H), 7.94 (d, J=8.0 Hz, 2H), 7.49 (d, J=10.1 Hz, 1H), 7.27 (d, J=8.0 Hz, 1H), 6.98 (m, 3H), 6.80 (s, 1H), 6.73-6.55 (m, 3H), 6.21 (s, 1H). ¹³C NMR (126 MHz, DMSO) δ 182.94, 182.28, 167.85, 164.11, 162.35, 161.66, 160.25, 159.31, 156.88, 156.37, 149.11, 144.85, 136.08, 128.96, 124.09, 121.62, 121.03, 120.38, 116.48, 114.90, 111.09, 110.10, 108.39, 107.41, 107.18, 103.96, 103.32, 93.85. HRMS (ESI-TOF) calcd for C₃₀H₁₇O₁₀=537.0827, found 537.0821.

### 6-(2,3-dihydroxy-6-(6-hydroxy-4-oxo-4H-chromen-2-yl)phenyl)-5,7-dihydroxy-2-(4-hydroxyphenyl)-4H-chromen-4-one (634-Ap (2-6), 2m)

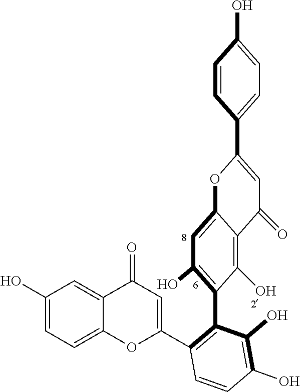

2m was prepared from Ap. 82 mg, 61% yield, brown solid. HPLC (ACN/Water=72.5:27.5, A=300 nm), tᵣ(2m)=14.506 min, concentration of 2m=13.71 mM. ¹H NMR (500 MHz, DMSO-d₆) δ 13.14 (s, 1H), 9.99 (s, 5H), 7.95 (d, J=8.2 Hz, 2H), 7.25-7.09 (m, 4H), 6.97 (dd, J=12.2, 8.2 Hz, 3H), 6.79 (s, 1H), 6.60 (s, 1H), 6.08 (s, 1H). ¹³C NMR (126 MHz, DMSO) δ 182.29, 177.02, 165.76, 164.10, 162.51, 161.66, 159.34, 156.84, 155.09, 149.96, 148.42, 144.75, 129.00, 125.09, 124.32, 123.31, 121.69, 120.49, 120.23, 119.60, 116.51, 114.83, 108.65, 108.13, 107.86, 103.92, 103.35, 93.83. HRMS (ESI-TOF) calcd for C₃₀H₁₇O₁₀=537.0827, found 537.0821.

### 8-(2,3-dihydroxy-6-(6-hydroxy-4-oxo-4H-chromen-2-yl)phenyl)-5,7-dihydroxy-2-(4-hydroxyphenyl)-4H-chromen-4-one (634-Ap (2′-8), 2m′)

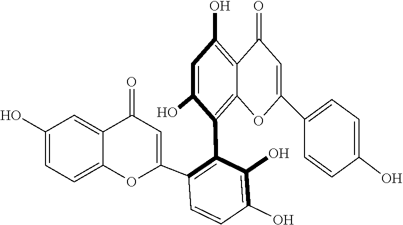

2m′ was prepared from Ap. 4 mg, 3% yield, brown solid. HPLC (ACN/Water=72.5:27.5, λ=300 nm), tᵣ(2m′)=12.177 min, concentration of 2m′=2.32 mM. ¹H NMR (500 MHz, DMSO-d₆) δ 12.98 (s, 1H), 9.94 (s, 5H), 7.52 (d, J=8.5 Hz, 2H), 7.25 (d, J=8.2 Hz, 1H), 7.16 (d, J=3.0 Hz, 1H), 7.10-7.01 (m, 2H), 6.87 (d, J=8.2 Hz, 1H), 6.79 (d, J=8.5 Hz, 2H), 6.71 (s, 1H), 6.26 (s, 1H), 5.99 (s, 1H). ¹³C NMR (126 MHz, DMSO) δ 182.38, 176.80, 165.90, 163.89, 161.52, 160.85, 155.03, 154.73, 149.67, 148.73, 145.16, 128.61, 125.16, 124.17, 123.18, 121.69, 120.64, 119.44, 119.23, 116.23, 114.96, 108.09, 107.79, 103.94, 103.79, 102.83, 99.07. HRMS (ESI-TOF) calcd for C₃₀H₁₇O₁₀=537.0827, found 537.0818.

### 6-(2,3-dihydroxy-6-(7-hydroxy-4-oxo-4H-chromen-2-yl)phenyl)-5,7-dihydroxy-2-(4-hydroxyphenyl)-4H-chromen-4-one (734-Ap (2′-6), 2n)

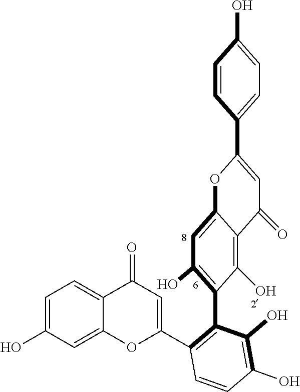

2n was prepared from Ap. 65 mg, 48% yield, brown solid. HPLC (ACN/Water=72.5:27.5, A=300 nm), tᵣ(2n)=14.399 min, concentration of 2n=22.53 mM. ¹H NMR (500 MHz, DMSO-d₆) δ 13.13 (s, 1H), 10.32 (s, 5H), 7.96 (d, J=8.5 Hz, 2H), 7.75 (d, J=8.5 Hz, 1H), 7.18 (d, J=8.2 Hz, 1H), 7.03-6.87 (m, 3H), 6.82 (d, J=8.2 Hz, 2H), 6.58 (s, 1H), 6.50 (s, 1H), 6.02 (s, 1H). ¹³C NMR (126 MHz, DMSO) δ 182.28, 176.51, 165.49, 164.02, 162.85, 162.41, 161.62, 159.28, 157.97, 156.77, 148.31, 144.68, 128.97, 126.80, 124.92, 121.68, 120.41, 120.13, 116.46, 116.22, 115.19, 114.74, 108.65, 108.59, 103.88, 103.34, 102.37, 93.76. HRMS (ESI-TOF) calcd for C₃₀H₁₇O₁₀=537.0827, found 537.0824.

### 8-(2,3-dihydroxy-6-(7-hydroxy-4-oxo-4H-chromen-2-yl)phenyl)-5,7-dihydroxy-2-(4-hydroxyphenyl)-4H-chromen-4-one (734-Ap (2-8), 2n′)

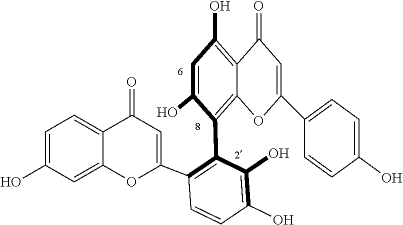

2n′ was prepared from Ap. 4 mg, 3% yield, brown solid. HPLC (ACN/Water=72.5:27.5, λ=300 nm), tᵣ(2n′)=12.139 min, concentration of 2n′=3.34 mM. ¹H NMR (500 MHz, DMSO-d₆) δ 12.98 (s, 1H), 10.42 (s, 5H), 7.70 (d, J=8.5 Hz, 1H), 7.52 (d, J=8.5 Hz, 2H), 7.23 (d, J=8.3 Hz, 1H), 7.02 (d, J=8.3 Hz, 1H), 6.78 (d, J=8.5 Hz, 3H), 6.72 (s, 1H), 6.26 (d, J=8.5 Hz, 1H), 6.25 (s, 1H), 5.95 (s, 1H). ¹³C NMR (126 MHz, DMSO) δ 182.38, 176.36, 165.70, 163.88, 162.78, 161.50, 160.81, 157.78, 154.71, 148.63, 145.08, 128.59, 126.76, 125.13, 121.71, 120.66, 119.37, 116.22, 116.08, 115.16, 114.93, 108.68, 103.93, 103.80, 102.85, 102.13, 99.03. HRMS (ESI-TOF) calcd for C₃₀H₁₇O₁₀=537.0827, found 537.0822.

### 6-(2,3-dihydroxy-6-(4-oxo-4H-chromen-2-yl)phenyl)-5,7-dihydroxy-2-(4-hydroxyphenyl)-4H-chromen-4-one (34-Ap (2-6), 2o)

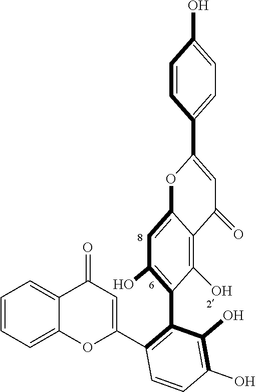

2o was prepared from Ap. 60 mg, 46% yield, brown solid. HPLC (ACN/Water=71.5:28.5, A=300 nm), tᵣ(2o)=31.427 min, concentration of 20=10.06 mM. ¹H NMR (500 MHz, DMSO-d₆) δ 13.16 (s, 1H), 10.36 (s, 4H), 7.93 (t, J=8.6 Hz, 3H), 7.68 (t, J=8.0 Hz, 1H), 7.42-7.32 (m, 1H), 7.32-7.18 (m, 2H), 6.99 (d, J=8.3 Hz, 1H), 6.95 (d, J=8.3 Hz, 2H), 6.79 (d, J=3.7 Hz, 1H), 6.60 (d, J=3.7 Hz, 1H), 6.15 (d, J=3.7 Hz, 1H). ¹³C NMR (126 MHz, DMSO) δ 182.26, 177.04, 166.14, 164.06, 162.44, 161.65, 159.33, 156.81, 156.18, 148.58, 144.76, 134.42, 128.97, 125.63, 125.14, 124.78, 123.41, 121.64, 120.59, 120.27, 118.24, 116.46, 114.83, 109.04, 108.56, 103.91, 103.32, 93.81. HRMS (ESI-TOF) calcd for C₃₀H₁₇O₉=521.0827, found 521.0871.

### Example 8. Reaction of luteolin with trihydroxyflavones (TFL) with catecholic group on B ring

2h-2o were prepared from one of 1a-1b (1 equiv) and one of 1h-1k (1.5 equiv), by following the protocol in Example 1.

2h

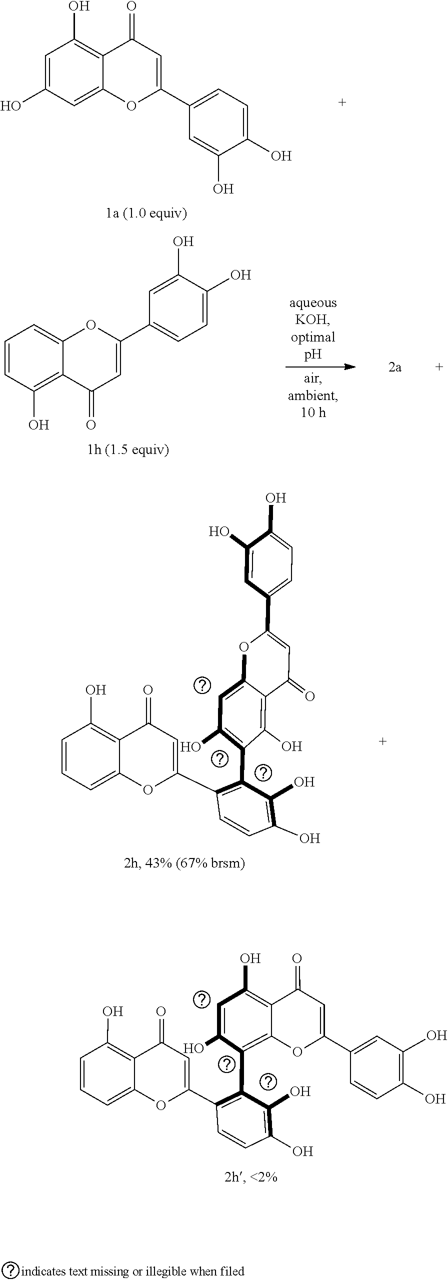

2h was prepared from 1a and 1h. 43% yield. Semi-prep HPLC (DI water with 0.1% formic acid/ACN with 0.1% formic acid, =64:36), t(2h)=14.273 min, t(2h′)=11.629 min.

2i

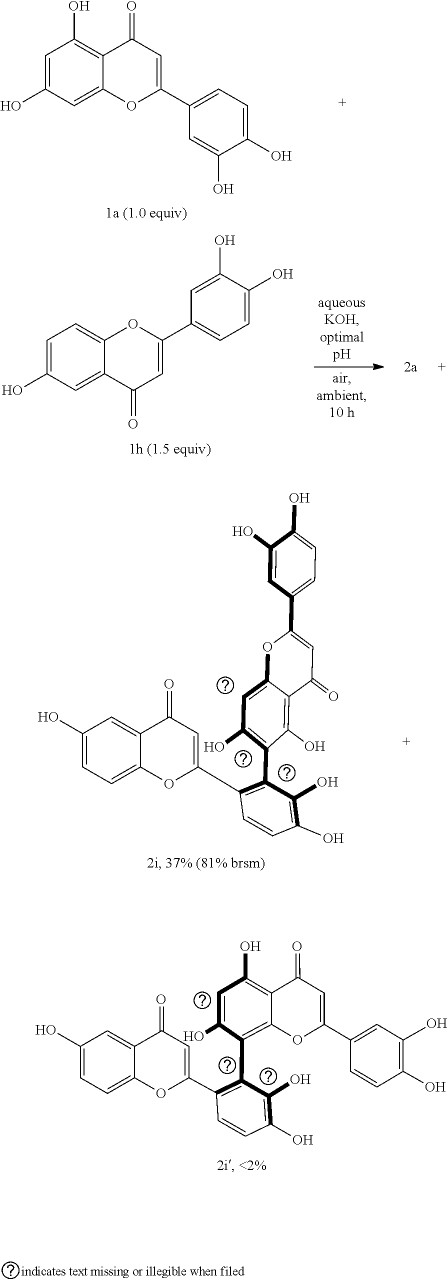

2i was prepared from 1a and 1i. 37/% yield. Semi-prep HPLC (DI water with 0.1% formic acid/ACN with 0.1% formic acid, 72.5:27.5), tᵣ(2i)=9.744 min

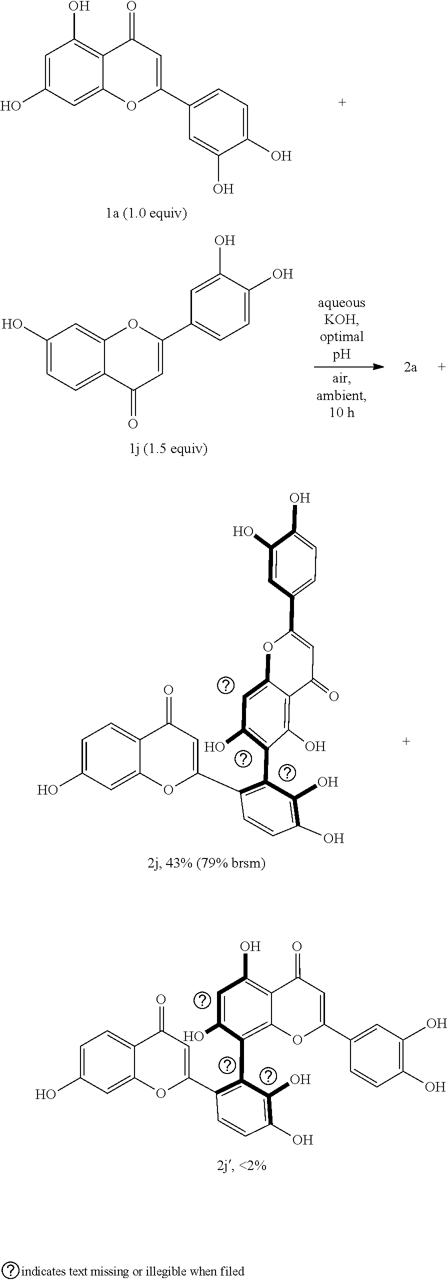

2j was prepared from 1a and 1j. 43% yield. Semi-prep HPLC (DI water with 0.1% formic acid/ACN with 0.1% formic acid, =72.5:27.5), tᵣ(2j)=9.707 min.

2k

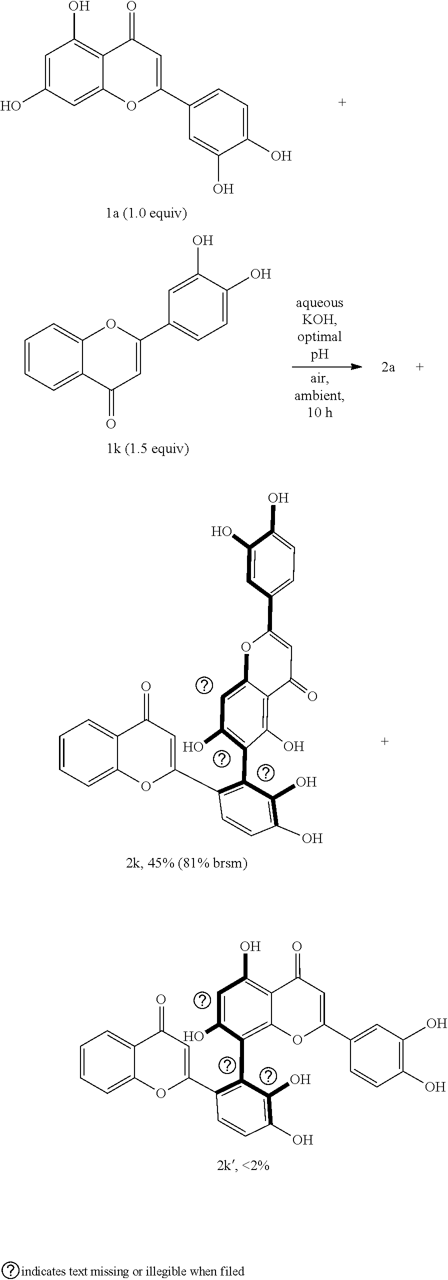

2k was prepared from 1a and 1k. 45% yield. Semi-prep HPLC (DI water with 0.1% formic acid/ACN with 0.1% formic acid, =71.5:28.5), tᵣ(2k)=21.725 min.

2l

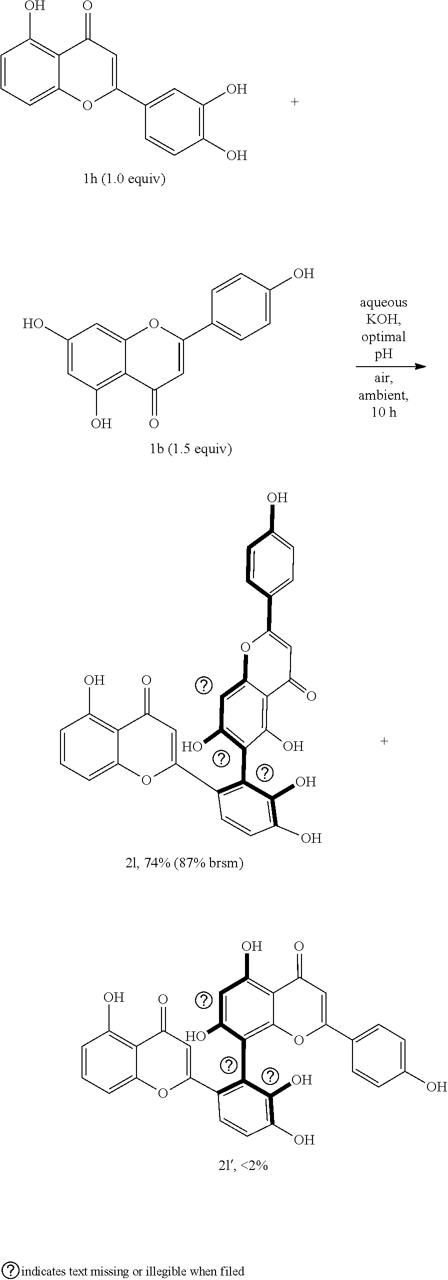

2l was prepared from 1h and 1b. 74% yield. Semi-prep HPLC (DI water with 0.1% formic acid/ACN with 0.1% formic acid, =64:36), tᵣ(21)=17.840 min, t(21′)=16.807 min.

2m

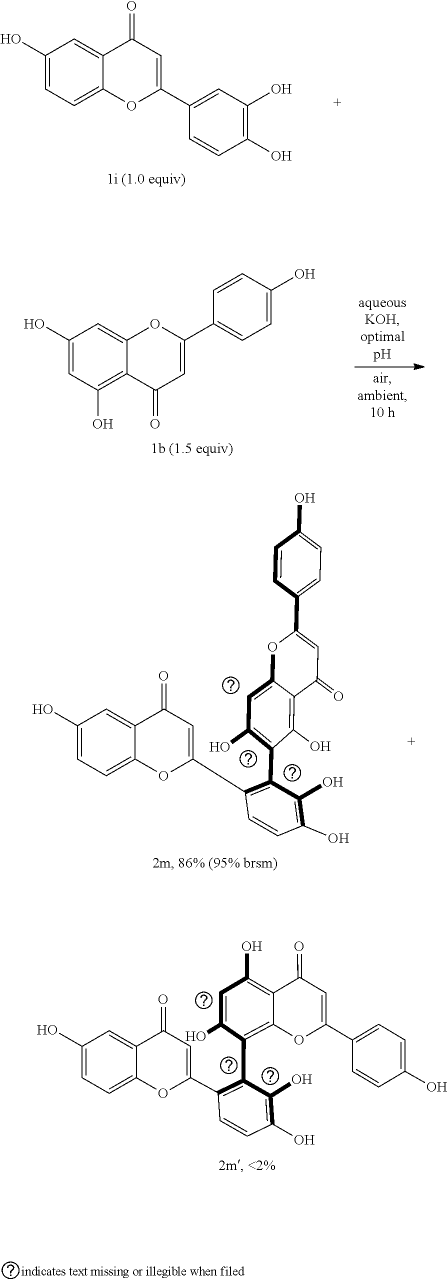

2m was prepared from 1 i and 1 b. 86% yield. Semi-prep HPLC (DI water with 0.1% formic acid/ACN with 0.1% formic acid, =72.5:27.5), tᵣ2m)=14.695 min, tᵣ2m′)=12.509 min.

2n

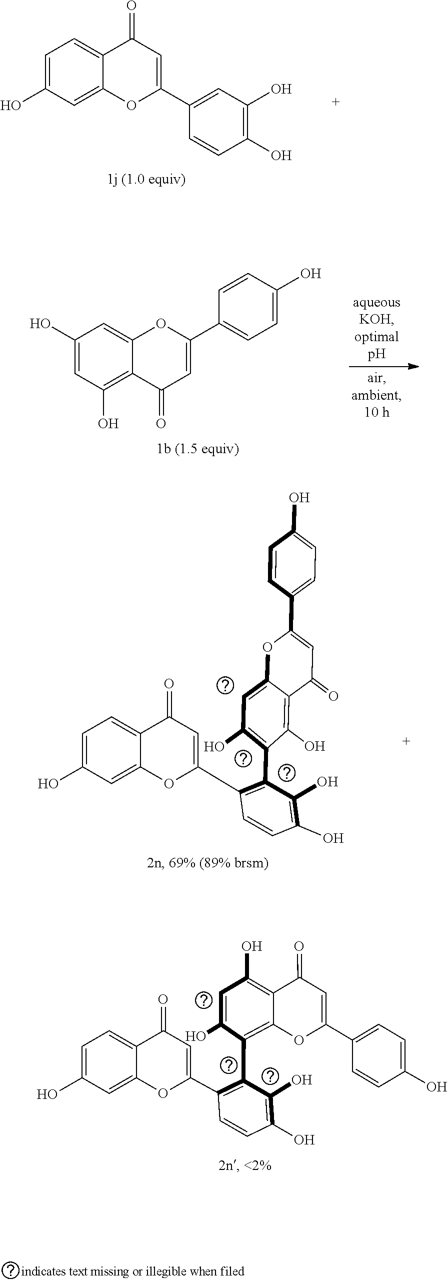

2n was prepared from 1j and 1b. 86% yield. Semi-prep HPLC (DI water with 0.1% formic acid/ACN with 0.1% formic acid, =72.5:27.5), tᵣ(2n)=13.003 min, tᵣ(2n′)=11.015 min.

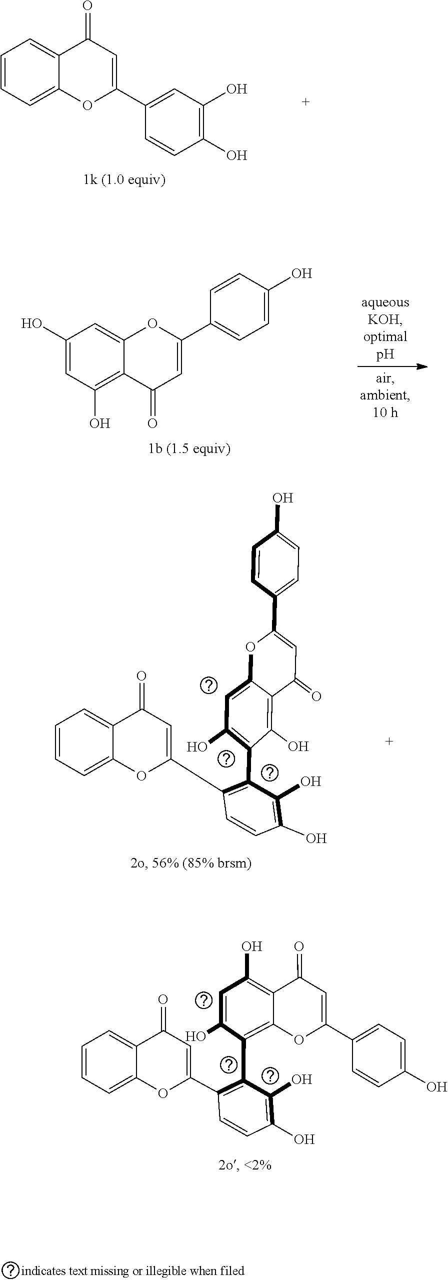

2o was prepared from 1k and 1b. 86% yield. Semi-prep HPLC (DI water with 0.1% formic acid/ACN with 0.1% formic acid, =71.5:28.5), tᵣ(2o)=30.602 min, tᵣ(20′)=28.932 min.

Results and Discussion

When luteolin was mixed with TFL with catecholic group on B ring (1h-1j) and 3′,4′-dihydroxyflavone (1k, DFL), luteolin became a nucleophile and 1h-1k was the radical precursor yielding corresponding biflavones FL-(2′-6)-Lu (2h-2k, FL=TFL and DFL, FIG. 10A). Not surprisingly, 1h-1k couples with other nucleophilic flavones such as Ap, forming FL-(2′-6)-Ap (2l, 2m, 2n, 2o) as the sole product. These results broaden the scope of our reaction to diverse biflavones containing two different monoflavones. There are many other feasible combinations of flavones and flavone glycosides that can meet this simple requirement.

### Example 9. Syntheses of Hetero Triflavonoids

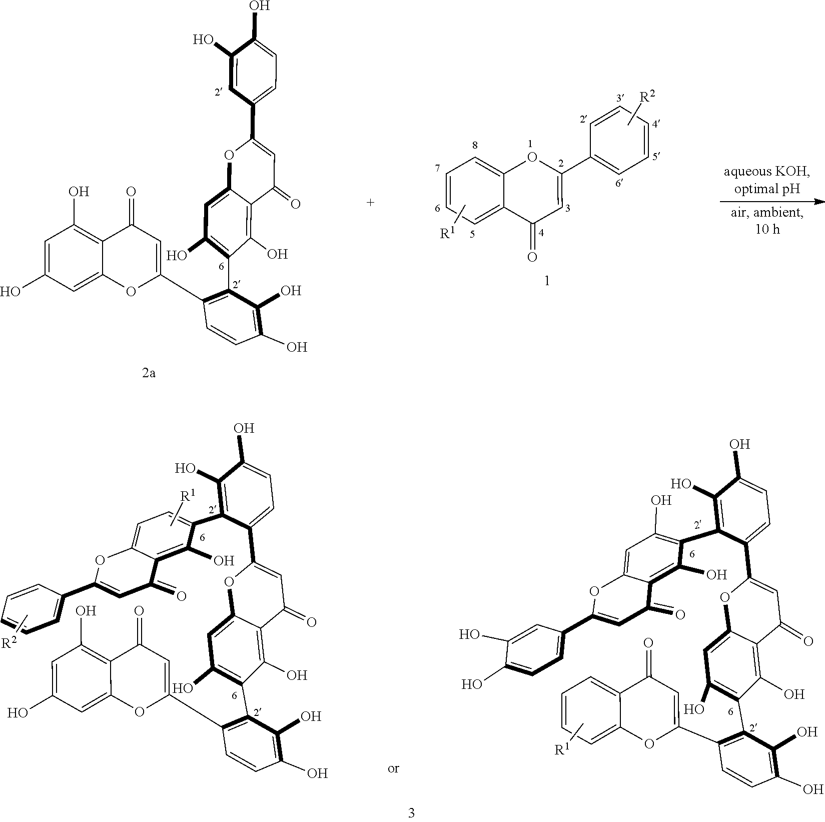

3b-3k were prepared from 2a (143 mg, 0.25 mmol) and a flavone (0.375 mmol) selected from Ap, Dio, Chry, 5,6-dihydroxyflavone, Ge, 5,3′,4′-trihydroxyflavone, 6,3′,4′-trihydroxyflavone, 7,3′,4′-trihydroxyflavone and 3′,4′-dihydroxyflavone, by following the protocol in Example 1.

### 2-(2-(5,7-dihydroxy-2-(4-hvdroxyphenyl)-4-oxo-4H-chromen-6-yl)-3,4-dihydroxyphenyl)-6-(6-(5,7-dihydroxy-4-oxo-4H-chromen-2-yl)-2,3-dihydroxyphenyl)-5,7-dihydroxy-4H-chromen-4-one (Lu₂-Ap (2-6), 3b)

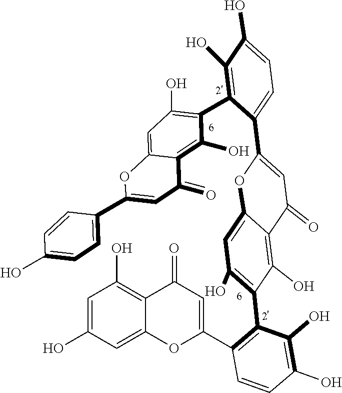

3b was prepared from Ap. 109 mg, 52% yield, brown solid. HPLC (ACN/Water=71.5:28.5, A=300 nm), tᵣ(3b*)=32.142 min, tᵣ(3b**)=33.539 min, concentration of 3b=0.89 mM. 3b=3b*+3b**. Atropisomer 3b*: ¹H NMR (500 MHz, DMSO-d₆) δ 7.90 (d, J=8.9, 2H), 7.22 (d, J=8.4 Hz, 1H), 7.15 (d, J=8.4 Hz, 1H), 6.97 (d, J=8.5 Hz, 1H), 6.94 (d, J=8.9 Hz, 2H), 6.92 (d, J=8.5 Hz, 1H), 6.69 (s, 1H), 6.57 (s, 1H), 6.21 (s, 1H), 6.09 (d, J=7.8 Hz, 1H), 6.01 (s, 1H), 5.98 (d, J=7.8 Hz, 1H), 5.97 (s, 1H). Atropisomer 3b**: ¹H NMR (500 MHz, DMSO-d₆) δ 7.90 (d, J=8.9 Hz, 2H), 7.22 (d, J=8.4, 1H), 7.15 (d, J=8.4 Hz, 1H), 6.97 (d, J=8.5 Hz, 1H), 6.94 (d, J=8.9 Hz, 2H), 6.92 (d, J=8.5 Hz, 1H), 6.68 (s, 1H), 6.55 (s, 1H), 6.16 (s, 1H), 6.09 (d, J=7.8 Hz, 1H), 5.99 (s, 1H), 5.98 (d, J=7.8 Hz, 1H), 5.96 (s, 1H). ¹³C NMR (126 MHz, DMSO) δ 182.34, 181.82, 181.69, 166.65, 166.56, 164.53, 164.07, 162.21, 161.76, 161.57, 159.35, 159.09, 157.88, 156.93, 148.82, 144.74, 128.98, 124.18, 121.77, 120.63, 120.15, 116.42, 114.78, 108.41, 108.25, 106.73, 103.97, 103.83, 103.65, 103.45, 99.20, 94.03, 93.86, 93.78. HRMS (ESI-TOF) calcd for C₄₅H₂₅O₁₇=837.1090, found 837.1097.

Crystals of compound 3b suitable for X-ray analysis were obtained by slow evaporation from MeOH. A brown Block-like specimen of C₃₆H₃₂O₁₄, approximate dimensions 0.061 mm×0.063 mm×0.267 mm, was used for the X-ray crystallographic analysis. The X-ray intensity data were measured. The total exposure time was 19.00 hours. The integration of the data using a triclinic unit cell yielded a total of 18025 reflections to a maximum θ angle of 67.110 (0.84 Å resolution), of which 5625 were independent (average redundancy 3.204, completeness=99.0%, Rᵢₙₜ=6.56%, R_{sig}=6.46%) and 3788 (67.34%) were greater than 2σ(F²). The final cell constants of a=8.4765 Å, b=11.1018 Å, c=18.6315 Å, α=98.306°, p=95.037°, γ=109.024°, volume=1623.0 Å³, are based upon the refinement of the XYZ-centroids of 49 reflections above 20 a(I) with 8.533°<2θ<40.75°. The ratio of minimum to maximum apparent transmission was 0.883. The structure was solved and refined using the Bruker SHELXTL Software Package, with Z=2 for the formula unit, C₃₆H₃₂O₁₄. The final anisotropic full-matrix least-squares refinement on F² with 491 variables converged at R1=4.71%, for the observed data and wR2=12.43% for all data. The goodness-of-fit was 1.029. The largest peak in the final difference electron density synthesis was 0.253 e⁻/Å³ and the largest hole was −0.240 e⁻/Å³ with an RMS deviation of 0.056 e⁻/Å³. On the basis of the final model, the calculated density was 1.434 g/cm³ and F(000), 720 e⁻. Crystallographic data have been deposited with the Cambridge Cystallograhic Data Centre.

### 2-(2-(5,7-dihydroxy-2-(4-hydroxy-3-methoxyphenyl)-4-oxo-4H-chromen-6-yl)-3,4-dihydroxyphenyl)-6-(6-(5,7-dihydroxy-4-oxo-4H-chromen-2-yl)-2,3-dihydroxyphenyl)-5,7-dihydroxy-4H-chromen-4-one (Lu₂-Dio (2-6), 3c)

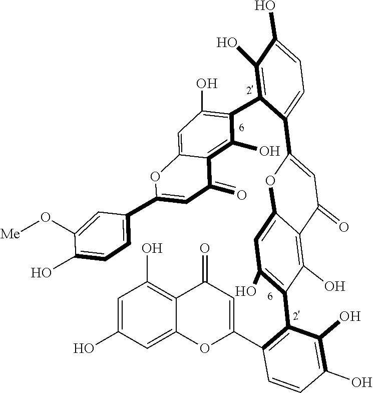

3c was prepared from Dio. 100 mg, 46% yield, brown solid. HPLC (ACN/Water=64:36, λ=300 nm), tᵣ(3c*)=10.787 min, tᵣ(3c**)=10.787 min, concentration of 3c=0.50 mM. 3c=3c*+3c**. Atropisomer 3c*: ¹H NMR (500 MHz, DMSO-d₆) δ 13.19 (s, 1H), 13.00 (s, 1H), 12.79 (s, 1H), 10.76 (s, 4H), 10.16 (s, 2H), 8.44 (s, 2H), 7.62 (d, J=8.6 Hz, 1H), 7.46 (m, 1H), 7.26 (d, J=8.4, 1H), 7.16 (d, J=8.7 Hz, 1H), 7.09 (d, J=8.6 Hz, 1H), 6.98 (d, J=8.5, 1H), 6.93 (d, J=8.4 Hz, 1H), 6.78 (s, 1H), 6.60 (s, 1H), 6.28 (s, 1H), 6.12 (d, J=8.1 Hz, 1H), 6.09 (s, 1H), 6.02 (d, J=8.1 Hz, 1H), 5.96 (s, 1H), 3.87 (s, 3H). Atropisomer 3c**: ¹H NMR (500 MHz, DMSO-d₆) δ 13.16 (s, 1H), 12.96 (s, 1H), 12.76 (s, 1H), 10.76 (s, 4H), 10.16 (s, 2H), 8.44 (s, 2H), 7.62 (d, J=8.6 Hz, 1H), 7.46 (m, 1H), 7.26 (d, J=8.4, 1H), 7.16 (d, J=8.7 Hz, 1H), 7.09 (d, J=8.6 Hz, 1H), 6.98 (d, J=8.5, 1H), 6.93 (d, J=8.4 Hz, 1H), 6.78 (s, 1H), 6.56 (s, 1H), 6.12 (d, J=8.1 Hz, 3H), 6.09 (s, 1H), 6.02 (d, J=8.1 Hz, 1H), 5.97 (s, 1H), 5.95 (s, 1H), 3.87 (s, 3H). ¹³C NMR (126 MHz, DMSO) δ 182.33, 182.26, 182.18, 181.86, 181.75, 181.69, 181.63, 166.68, 166.63, 166.58, 166.54, 166.28, 164.67, 164.53, 164.48, 164.01, 163.87, 163.83, 162.27, 162.21, 162.07, 161.95, 161.77, 159.38, 159.32, 159.09, 157.89, 157.85, 157.80, 157.10, 156.93, 156.83, 151.61, 151.58, 151.55, 148.85, 148.79, 148.70, 147.26, 147.20, 144.73, 144.69, 144.63, 124.19, 124.06, 123.60, 123.57, 123.48, 120.83, 120.65, 120.19, 120.14, 120.10, 119.24, 119.20, 114.82, 114.74, 113.48, 113.42, 113.37, 112.65, 112.61, 112.55, 108.45, 108.32, 108.25, 108.18, 106.84, 106.76, 106.66, 104.24, 104.13, 104.06, 104.00, 103.95, 103.86, 103.82, 103.68, 103.62, 99.36, 99.31, 99.20, 94.40, 94.35, 93.90, 93.85, 93.80, 93.47, 93.29, 56.22, 56.17. HRMS (ESI-TOF) calcd for C₄₆H₂₇O₁₈=867.1203, found 867.1202.

### 2-(2-(5,7-dihydroxy-4-oxo-2-phenyl-4H-chromen-6-yl)-3,4-dihydroxyphenyl)-6-(6-(5,7-dihydroxy-4-oxo-4H-chromen-2-yl)-2,3-dihydroxyphenyl)-5,7-dihydroxy-4H-chromen-4-one (Lu₂—Chry (2-6), 3d)

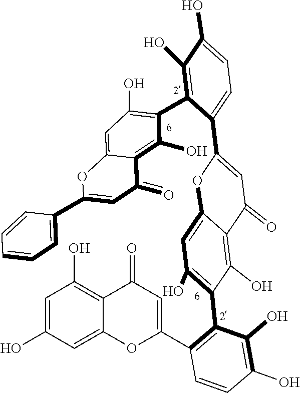

3d was prepared from Chry. 82 mg, 40% yield, brown solid. HPLC (ACN/Water=64:36, λ=300 nm), tᵣ(3d*)=9.863 min, tᵣ(3d**)=9.863 min, concentration of 3d=2.13 mM. 3d=3d*+3d**. Atropisomer 3d*: ¹H NMR (500 MHz, DMSO-d₆) δ 8.07-8.00 (m, 2H), 7.65-7.53 (m, 3H), 7.23 (d, J=8.4, Hz, 1H), 7.14 (dd, J=8.3, 1H), 6.99 (d, J=8.4 Hz, 1H), 6.93 (d, J=8.4 Hz, 1H), 6.85 (s, 1H), 6.65 (s, 1H), 6.24 (s, 1H), 6.11 (d, J=6.1 Hz, 1H), 6.06 (s, 1H), 6.02 (d, J=6.1 Hz, 1H), 5.96 (s, 1H). Atropisomer 3d**: ¹H NMR (500 MHz, DMSO-d₆) δ 8.07-8.00 (m, 2H), 7.65-7.53 (m, 3H), 7.23 (d, J=8.4 Hz, 1H), 7.14 (d, J=8.3 Hz, 1H), 6.99 (d, J=8.4 Hz, 1H), 6.93 (d, J=8.3 Hz, 1H), 6.85 (s, 1H), 6.63 (s, 1H), 6.16 (s, 1H), 6.11 (d, J=6.1 Hz, 1H), 6.02 (s, 1H), 6.01 (d, J=6.1 Hz, 1H), 5.94 (s, 1H). ¹³C NMR (126 MHz, DMSO) δ 182.48, 181.85, 181.69, 181.67, 166.67, 166.62, 166.32, 164.58, 163.57, 163.52, 162.20, 161.75, 159.32, 159.08, 159.04, 157.89, 157.08, 156.98, 156.93, 148.79, 148.70, 144.72, 144.62, 132.44, 131.28, 129.62, 126.93, 124.17, 120.64, 120.12, 120.08, 114.87, 114.77, 108.68, 108.54, 108.26, 108.20, 106.73, 105.77, 104.23, 103.82, 103.79, 103.65, 99.22, 93.89, 93.29. HRMS (ESI-TOF) calcd for C₄₅H₂₅O₁ₛ=821.1148, found 821.1135.

### 6-(6-(6-(6-(5,7-dihydroxy-4-oxo-4H-chromen-2-yl)-2,3-dihydroxyphenyl)-5,7-dihydroxy-4-oxo-4H-chromen-2-yl)-2,3-dihydroxyphenyl)-5,7-dihydroxy-8-methoxy-2-phenyl-4H-chromen-4-one (Lug-Wo (2′-6), 3e)

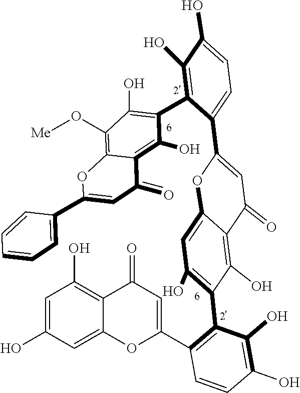

3e was prepared from Wo. 115 mg, 54% yield, brown solid. HPLC (ACN/Water=64:36, λ=300 nm), tᵣ(3e*)=21.393 min, tᵣ(3e**)=22.891 min, concentration of 3e=1.90 mM. 3e=3e*+3e**. Atropisomer 3e*: ¹H NMR (500 MHz, DMSO-d₆) δ 12.96 (s, 1H), 12.79 (s, 1H), 12.75 (s, 1H), 10.77 (s, 1H), 10.63 (s, 1H), 10.31 (s, 1H), 10.26 (s, 1H), 8.51 (s, 1H), 8.38 (s, 1H), 8.09 (d, J=7.7 Hz, 2H), 7.68-7.56 (m, 3H), 7.26 (d, J=8.4, 1H), 7.14 (d, J=8.4 Hz, 1H), 7.04 (s, 1H), 6.98 (d, J=8.4, 1H), 6.91 (d, J=8.4, 1H), 6.15 (s, 1H), 6.12 (d, J=2.1 Hz, 1H), 6.04 (d, J=2.1 Hz, 1H), 6.01 (s, 1H), 5.95 (s, 1H). Atropisomer 3e**: ¹H NMR (500 MHz, DMSO-d₆) δ 12.96 (s, 1H), 12.77 (s, 1H), 12.74 (s, 1H), 10.77 (s, 1H), 10.57 (s, 1H), 10.27 (s, 1H), 10.12 (s, 1H), 8.51 (s, 1H), 8.35 (s, 1H), 8.09 (d, J=7.7 Hz, 2H), 7.68-7.56 (m, 3H), 7.26 (d, J=8.4, 1H), 7.14 (d, J=8.4 Hz, 1H), 7.04 (s, 1H), 6.98 (d, J=8.4, 1H), 6.91 (d, J=8.4, 1H), 6.11 (s, 1H), 6.08 (d, J=2.1 Hz, 1H), 5.97 (s, 1H), 5.95 (d, J=2.1 Hz, 1H), 5.92 (s, 1H). ¹³C NMR (126 MHz, DMSO) δ 182.69, 182.59, 181.90, 181.78, 181.68, 181.64, 166.70, 166.59, 166.36, 164.47, 163.33, 162.21, 162.12, 161.76, 159.12, 159.00, 157.92, 157.84, 157.01, 156.78, 155.40, 154.58, 149.11, 148.88, 148.68, 144.76, 144.62, 132.52, 131.41, 129.75, 127.99, 127.87, 126.78, 124.30, 124.15, 124.01, 123.93, 120.74, 120.58, 120.14, 119.80, 114.91, 114.70, 108.86, 108.74, 108.20, 106.83, 106.71, 105.77, 103.90, 103.81, 103.64, 99.16, 93.93, 93.80, 93.38, 93.24, 61.97. HRMS (ESI-TOF) calcd for C₄₆H₂₇O₁₇=851.1254, found 851.1248.

Crystals of compound 3e suitable for X-ray analysis were obtained by slow evaporation from MeOH and DMSO. A specimen of C₃₅H₂₆N₂O₁₁, approximate dimensions 0.049 mm×0.122 mm×0.132 mm, was used for the X-ray crystallographic analysis. The X-ray intensity data were measured (λ=1.54178 Å). The total exposure time was 17.13 hours. The integration of the data using a triclinic unit cell yielded a total of 8055 reflections to a maximum θ angle of 67.03° (0.84 Å resolution), of which 8055 were independent (average redundancy 1.000, completeness=97.6%, R_{sig}=10.51%) and 4577(56.82%) were greater than 2σ(F²). The final cell constants of a=8.6271(6) Å, b=10.2328(6) Å, c=17.4619(12) Å, α=102.701(4°), β=102.737(4°), γ=90.020(4°), volume=1464.87(17) Å³, are based upon the refinement of the XYZ-centroids of 4286 reflections above 20 σ(I) with 8.870°<2θ<133.1°. The ratio of minimum to maximum apparent transmission was 0.637. The calculated minimum and maximum transmission coefficients (based on crystal size) were 0.4799 and 0.7528. The structure was solved and refined using the Bruker SHELXTL Software Package, using the space group P−1, with Z=2 for the formula unit, C₃₅H₂₆N₂O₁₁. The final anisotropic full-matrix least-squares refinement on F² with 455 variables converged at R1=9.67%, for the observed data and wR2=32.15% for all data. The goodness-of-fit was 1.065. The largest peak in the final difference electron density synthesis was 0.495 e⁻/Å³ and the largest hole was −0.499 e⁻/Å³ with an RMS deviation of 0.104 e⁻/Å³. On the basis of the final model, the calculated density was 1.475 g/cm³ and F(000), 676 e⁻. Crystallographic data have been deposited with the Cambridge Crystallographic Data Centre.

### 2-(2-(5,6-dihydroxy-4-oxo-2-phenyl-4H-chromen-7-yl)-3,4-dihydroxyphenyl)-6-(6-(5,7-dihydroxy-4-oxo-4H-chromen-2-yl)-2,3-dihydroxyphenyl)-5,7-dihydroxy-4H-chromen-4-one (Lu₂-56 (2′-6), 3f)

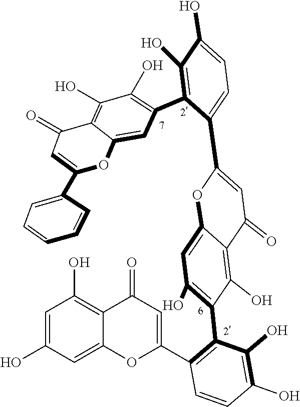

3f was prepared from 5,6-dihydroxyflavone. 154 mg, 75% yield, brown solid. HPLC (ACN/Water=64:36, λ=300 nm), tᵣ(3f*)=9.731 min, tᵣ(3f**)=9.731 min, concentration of 3f=3.50 mM. 3f=3f*+3f**. Atropisomer 3f*: ¹H NMR (400 MHz, DMSO-d₆) δ 7.67 (d, J=7.1 Hz, 1H), 7.59 (d, J=7.1 Hz, 1H), 7.50 (dt, J=7.1, 3.8 Hz, 1H), 7.46-7.39 (m, 2H), 7.38 (d, J=6.9 Hz, 1H), 7.33 (d, J=7.1 Hz, 1H), 7.24 (s, 1H), 7.07 (s, 1H), 6.90 (d, J=3.8 Hz, 1H), 6.70 (s, 2H), 6.60 (d, J=6.9 Hz, 1H), 6.44 (d, J=7.1 Hz, 1H), 6.37 (s, 1H), 5.89 (s, 1H). Atropisomer 3f**: ¹H NMR (400 MHz, DMSO-d₆) δ 7.67 (d, J=7.1 Hz, 1H), 7.59 (d, J=7.1 Hz, 1H), 7.50 (dt, J=7.1, 3.8 Hz, 1H), 7.46-7.39 (m, 2H), 7.38 (d, J=6.9 Hz, 1H), 7.33 (d, J=7.1 Hz, 1H), 7.34 (s, 1H), 6.93 (s, 1H), 6.90 (d, J=3.8 Hz, 1H), 6.70 (s, 2H), 6.60 (d, J=6.9 Hz, 1H), 6.44 (d, J=7.1 Hz, 1H), 6.43 (s, 1H), 5.81 (s, 1H). ¹³C NMR (126 MHz, DMSO) δ 184.34, 184.25, 182.02, 181.89, 181.84, 166.66, 166.04, 164.17, 164.08, 164.00, 163.95, 162.39, 162.31, 162.26, 161.65, 161.30, 159.52, 158.94, 156.81, 156.70, 155.37, 155.31, 150.17, 150.11, 148.78, 148.75, 146.74, 146.68, 146.49, 146.45, 146.16, 144.66, 144.62, 141.05, 140.92, 132.66, 132.57, 131.22, 129.61, 129.53, 126.44, 126.41, 126.32, 126.12, 124.09, 123.87, 121.97, 121.92, 120.21, 120.18, 119.92, 119.49, 119.41, 116.48, 114.37, 113.78, 111.13, 111.08, 110.67, 110.56, 107.74, 107.47, 107.00, 106.81, 104.88, 103.97, 103.93, 103.84, 103.78, 103.41, 103.29, 102.23, 102.17, 98.99, 93.80, 93.48. HRMS (ESI-TOF) calcd for C₄₅H₂₅O₁₆=821.1148, found 821.1133.

### 2-(2-(5,7-dihydroxy-3-(4-hydroxyphenyl)-4-oxo-4H-chromen-6-yl)-3,4-dihydroxyphenyl)-6-(6-(5,7-dihydroxy-4-oxo-4H-chromen-2-yl)-2,3-dihydroxyphenyl)-5,7-dihydroxy-4H-chromen-4-one (Lu₂-Ge (2′-6), 3q)

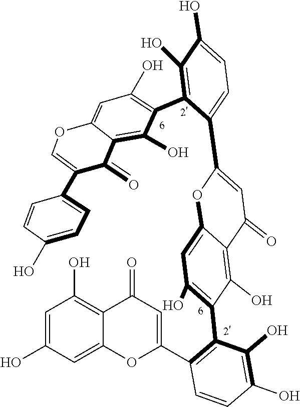

3g was prepared from Ge. 103 mg, 49% yield, brown solid. HPLC (ACN/Water=64:36, λ=300 nm), tᵣ(3g*)=10.218 min, tᵣ(3g**)=11.126 min, concentration of 3g=3.88 mM. 3g=3g*+3g**. Atropisomer 3g*: ¹H NMR (400 MHz, DMSO-d₆) δ 8.31 (s, 1H), 7.39 (d, J=8.2 Hz, 2H), 7.25 (d, J=8.4 Hz, 1H), 7.18 (d, J=8.3 Hz, 1H), 6.98 (d, J=8.4 Hz, 1H), 6.94 (d, J=8.3 Hz, 1H), 6.83 (d, J=8.2 Hz, 2H), 6.52 (s, 1H), 6.28 (s, 1H), 6.13 (d, J=2.1 Hz, 1H), 6.09 (s, 1H), 6.04 (d, J=2.1 Hz, 1H), 5.96 (s, 1H). Atropisomer 3g**: ¹H NMR (400 MHz, DMSO-d₆) δ 8.34 (s, 1H), 7.42 (d, J=8.4 Hz, 2H), 7.25 (d, J=8.2 Hz, 1H), 7.17 (d, J=8.3 Hz, 1H), 6.97 (d, J=8.2 Hz, 1H), 6.93 (d, J=8.3 Hz, 1H), 6.83 (d, J=8.2 Hz, 2H), 6.48 (s, 1H), 6.28 (s, 1H), 6.11 (d, J=2.1 Hz, 1H), 6.09 (s, 1H), 6.01 (d, J=2.1 Hz, 1H), 5.97 (s, 1H). ¹³C NMR (126 MHz, DMSO) δ 181.86, 181.75, 181.68, 180.85, 180.77, 166.67, 166.62, 166.60, 166.32, 164.51, 164.49, 162.42, 162.21, 162.17, 161.78, 161.75, 159.86, 159.75, 159.10, 157.90, 157.85, 157.83, 157.17, 157.09, 157.06, 156.91, 154.27, 148.82, 148.77, 148.71, 148.68, 144.70, 144.66, 144.64, 144.61, 130.73, 130.70, 130.63, 124.23, 124.17, 124.11, 122.84, 122.80, 121.86, 121.78, 120.66, 120.18, 120.12, 115.52, 115.49, 114.83, 108.51, 108.38, 108.22, 106.89, 106.79, 106.73, 104.77, 104.74, 103.86, 103.83, 103.68, 99.19, 93.89, 93.84, 93.62, 93.48, 93.27. HRMS (ESI-TOF) calcd for C₄₅H₂₅O₁₇=837.1097, found 837.1087.

### 6-(2,3-dihydroxy-6-(5-hydroxy-4-oxo-4H-chromen-2-yl)phenyl)-2-(2-(2-(3,4-dihydroxyphenyl)-5,7-dihydroxy-4-oxo-4H-chromen-6-yl)-3,4-dihydroxyphenyl)-5,7-dihydroxy-4H-chromen-4-one (534-Lu₂ (2-6), 3h)

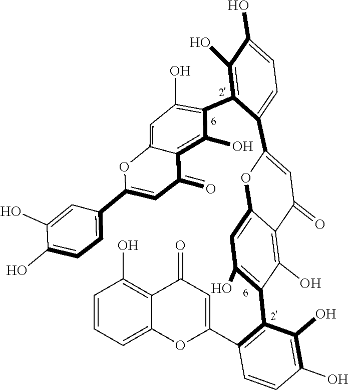

3h was prepared from 5,3′,4′-trihydroxyflavone. 88 mg, 42% yield, brown solid. HPLC (ACN/Water=64:36, λ=300 nm), tᵣ(3h*)=11.579 min, t4(3h**)=12.618 min, concentration of 3h=3.43 mM. 3h=3h*+3h**. Atropisomer 3h*: ¹H NMR (500 MHz, DMSO-d₆) δ 7.46 (td, J=8.3, 3.8 Hz, 1H), 7.44-7.37 (m, 2H), 7.22 (d, J=8.3 Hz, 1H), 7.21 (d, J=8.2 Hz, 1H), 6.98 (d, J=8.3, 1H), 6.95 (d, J=8.2 Hz, 1H), 6.93 (d, J=8.2 Hz, 1H), 6.67 (d, J=3.8 Hz, 1H), 6.61 (s, 1H), 6.58 (s, 1H), 6.52 (d, J=8.3 Hz, 1H), 6.24 (s, 1H), 6.15 (s, 1H), 6.08 (s, 1H). Atropisomer 3h**: ¹H NMR (500 MHz, DMSO-d₆) δ 7.46 (td, J=8.3, 3.8 Hz, 1H), 7.44-7.37 (m, 2H), 7.22 (d, J=8.3, 1H), 7.21 (d, J=8.2 Hz, 1H), 6.98 (d, J=8.3 Hz, 1H), 6.95 (d, J=8.2 Hz, 1H), 6.93 (d, J=8.2 Hz, 1H), 6.67 (d, J=8.1 Hz, 1H), 6.60 (s, 1H), 6.57 (s, 1H), 6.46 (d, J=8.1 Hz, 1H), 6.19 (s, 1H), 6.15 (s, 1H), 6.02 (s, 1H). ¹³C NMR (126 MHz, DMSO) δ 182.91, 182.86, 182.25, 182.21, 181.84, 181.74, 167.83, 166.72, 166.46, 164.20, 164.16, 162.25, 162.19, 162.05, 160.20, 159.27, 159.05, 159.00, 157.07, 156.91, 156.85, 156.77, 156.24, 156.19, 150.13, 150.08, 149.05, 148.81, 148.73, 146.18, 144.72, 144.68, 136.03, 135.97, 124.27, 124.05, 123.87, 123.80, 122.05, 122.00, 121.06, 120.90, 120.67, 120.27, 120.21, 120.17, 119.47, 116.52, 114.82, 113.79, 111.10, 110.08, 110.03, 108.42, 108.34, 108.27, 108.22, 107.21, 107.13, 107.05, 106.97, 106.85, 106.60, 103.95, 103.65, 103.62, 103.44, 93.73, 93.37, 93.24. HRMS (ESI-TOF) calcd for C₄₅H₂₅O₁₇=837.1097, found 837.1091.

### 6-(2,3-dihydroxy-6-(6-hydroxy-4-oxo-4H-chromen-2-yl)phenyl)-2-(2-(2-(3,4-dihydroxyphenyl)-5,7-dihydroxy-4-oxo-4H-chromen-6-yl)-3,4-dihydroxyphenyl)-5,7-dihydroxy-4H-chromen-4-one (634-Lu₂ (2-6), 3i)

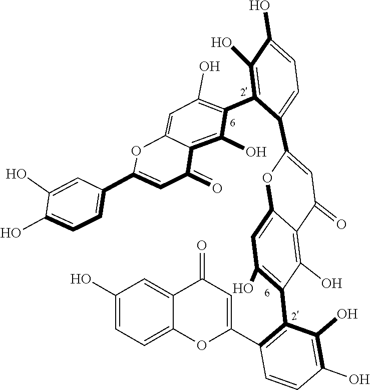

3i was prepared from 6,3′,4′-trihydroxyflavone. 85 mg, 41% yield, brown solid. HPLC (ACN/Water=72.5:27.5, λ=300 nm), tᵣ(3i*)=10.218 min, tᵣ(3i**)=11.126 min, concentration of 3i=3.73 mM. 3i=3i*+3i**. Atropisomer 3i*: ¹H NMR (400 MHz, DMSO-d₆) δ 7.44 (d, J=8.5 Hz, 2H), 7.24 (d, J=8.4, 1H), 7.21 (d, J=8.3, 1H), 7.17-7.03 (m, 3H), 6.96 (d, J=8.4, 1H), 6.93 (d, J=8.3, 1H), 6.90 (d, J=8.5 Hz, 1H), 6.70 (s, 1H), 6.56 (s, 1H), 6.24 (s, 1H), 6.02 (s, 1H), 5.93 (s, 1H). Atropisomer 3i**: ¹H NMR (400 MHz, DMSO-d₆) δ 7.44 (d, J=8.5 Hz, 2H), 7.24 (d, J=8.4, 11H), 7.21 (d, J=8.3, 1H), 7.17-7.03 (m, 3H), 6.96 (d, J=8.4, 1H), 6.93 (d, J=8.3, 1H), 6.90 (d, J=8.5 Hz, 1H), 6.70 (s, 1H), 6.54 (s, 1H), 6.09 (s, 1H), 6.05 (s, 1H), 5.97 (s, 1H). ¹³C NMR (126 MHz, DMSO) δ 182.28, 182.23, 181.80, 181.72, 176.92, 176.89, 166.52, 166.31, 165.61, 164.24, 164.19, 162.33, 162.17, 161.97, 159.36, 159.32, 159.10, 159.06, 157.03, 156.89, 156.80, 155.05, 150.13, 150.09, 149.87, 149.83, 148.83, 148.77, 148.30, 146.19, 144.71, 144.68, 144.58, 124.91, 124.88, 124.28, 124.23, 124.18, 124.02, 123.25, 122.09, 122.06, 120.81, 120.65, 120.41, 120.19, 120.08, 120.04, 119.50, 119.44, 116.52, 114.81, 113.87, 108.50, 108.39, 108.28, 107.97, 107.92, 107.82, 107.79, 106.76, 106.58, 103.99, 103.62, 103.48, 93.80, 93.74, 93.38, 93.26. HRMS (ESI-TOF) calcd for C₄₅H₂₅O₁₇=837.1097, found 837.1091.

### 6-(2,3-dihydroxy-6-(7-hydroxy-4-oxo-4H-chromen-2-yl)phenyl)-2-(2-(2-(3,4-dihydroxyphenyl)-5,7-dihydroxy-4-oxo-4H-chromen-6-yl)-3,4-dihydroxyphenyl)-5,7-dihydroxy-4H-chromen-4-one (734-Lu₂ (2-6), 3j)

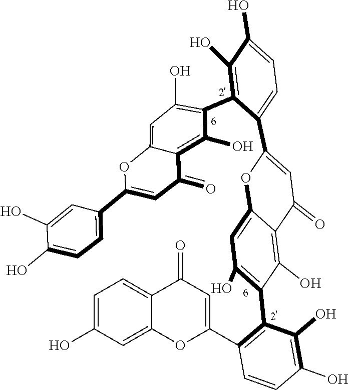

3j was prepared from 7,3′,4′-trihydroxyflavone. 98 mg, 47% yield, brown solid. HPLC (ACN/Water=72.5:27.5, λ=300 nm), tᵣ(3j*)=13.331 min, tᵣ(3j**)=13.331 min, concentration of 3j=2.54 mM. 3j=3j*+3j**. Atropisomer 3j*: ¹H NMR (400 MHz, DMSO-d₆) δ 7.73 (td, J=8.7, 6.5 Hz, 1H), 7.49 (d, J=8.1 Hz, 2H), 7.24 (d, J=8.4 Hz, 1H), 7.14 (d, J=8.3 Hz, 1H), 6.97 (d, J=8.4 Hz, 1H), 6.93 (d, J=8.3 Hz, 1H), 6.92 (d, J=8.1 Hz, 1H), 6.81 (dd, J=8.7, 6.5 Hz, 1H), 6.71 (s, 1H), 6.56 (s, 1H), 6.52 (d, J=6.5 Hz, 1H), 6.27 (s, 1H), 6.05 (s, 1H), 5.90 (s, 1H), 3.18 (s, 1H). Atropisomer 3j**: ¹H NMR (400 MHz, DMSO-d₆) δ 7.73 (td, J=8.7, 6.5 Hz, 1H), 7.49 (d, J=8.1 Hz, 2H), 7.24 (d, J=8.4 Hz, 1H), 7.14 (d, J=8.3 Hz, 1H), 6.97 (d, J=8.4 Hz, 1H), 6.93 (d, J=8.3 Hz, 1H), 6.92 (d, J=8.1 Hz, 1H), 6.81 (dd, J=8.8, 6.5 Hz, 1H), 6.70 (s, 1H), 6.54 (s, 1H), 6.52 (d, J=6.5 Hz, 1H), 6.09 (s, 1H), 5.93 (s, 1H), 5.92 (s, 1H), 3.18 (s, 1H). ¹³C NMR (126 MHz, DMSO) δ 206.96, 182.26, 182.19, 181.80, 181.71, 176.37, 166.50, 166.20, 165.23, 165.15, 164.20, 164.16, 162.87, 162.28, 162.15, 161.95, 159.36, 159.31, 159.11, 157.97, 157.04, 156.90, 156.86, 156.77, 150.11, 150.07, 148.80, 148.74, 148.22, 148.18, 146.18, 144.68, 144.64, 144.52, 126.76, 126.74, 124.88, 124.20, 124.08, 122.08, 122.04, 120.27, 120.21, 120.06, 120.02, 119.47, 116.53, 116.18, 116.14, 115.19, 114.84, 113.89, 108.57, 108.40, 108.32, 108.21, 106.83, 106.66, 103.99, 103.96, 103.64, 103.45, 102.41, 93.85, 93.73, 93.30. HRMS (ESI-TOF) calcd for C₄₅H₂₅O₁₇=837.1097, found 837.1086.

### 6-(2,3-dihydroxy-6-(7-hydroxy-4-oxo-4H-chromen-2-yl)phenyl)-2-(2-(2-(3,4-dihydroxyphenyl)-5,7-dihydroxy-4-oxo-4H-chromen-6-yl)-3,4-dihydroxyphenyl)-5,7-dihydroxy-4H-chromen-4-one (734-Lu-Ap (2-6), 3k)

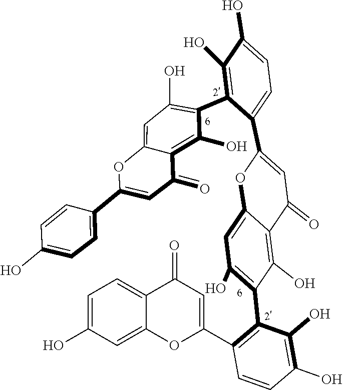

3k was prepared from 7,3′,4′-trihydroxyflavone. 88 mg, 43% yield, brown solid. HPLC (ACN/Water=71.5:28.5, λ=300 nm), tᵣ(3k)=15.699 min, concentration of 3k=0.76 mM. 3k=3k*+3k**. Atropisomer 3k*: ¹H NMR (500 MHz, DMSO-d₆) δ 13.20 (d, J=13.1 Hz, 1H), 12.94 (d, J=20.1 Hz, 1H), 10.37 (s, 8H), 7.94 (d, J=8.4 Hz, 2H), 7.71 (d, J=8.4 Hz, 1H), 7.22 (d, J=8.8 Hz, 1H), 7.13 (d, J=7.9 Hz, 1H), 6.93 (q, J=10.4 Hz, 3H), 6.78 (t, J=5.9 Hz, 2H), 6.53 (s, 1H), 6.44 (s, 1H), 6.22 (s, 1H), 5.98 (s, 1H), 5.86 (s, 1H). Atropisomer 3k**: ¹H NMR (500 MHz, DMSO-d₆) δ 13.20 (d, J=13.1 Hz, 1H), 12.94 (d, J=20.1 Hz, 1H), 10.37 (s, 8H), 7.94 (d, J=8.4 Hz, 2H), 7.71 (d, J=8.4 Hz, 1H), 7.22 (d, J=8.8 Hz, 1H), 7.13 (d, J=7.9 Hz, 1H), 6.93 (q, J=10.4 Hz, 3H), 6.78 (t, J=5.9 Hz, 2H), 6.53 (s, 1H), 6.44 (s, 1H), 6.12 (s, 1H), 5.91 (s, 1H), 5.89 (s, 1H). ¹³C NMR (126 MHz, DMSO) δ 182.23, 181.77, 176.41, 166.57, 166.38, 165.29, 163.97, 162.50, 161.55, 159.38, 159.28, 159.10, 157.99, 156.96, 148.81, 148.26, 144.76, 144.62, 128.96, 126.68, 126.67, 124.76, 124.20, 121.80, 120.55, 120.24, 120.12, 116.41, 114.64, 108.47, 108.38, 106.67, 106.66, 103.54, 103.39, 102.35, 102.32, 93.97, 93.37. HRMS (ESI-TOF) calcd for C₄₅H₂₅O₁ₛ=821.1148, found 821.1132.

Results and Discussion

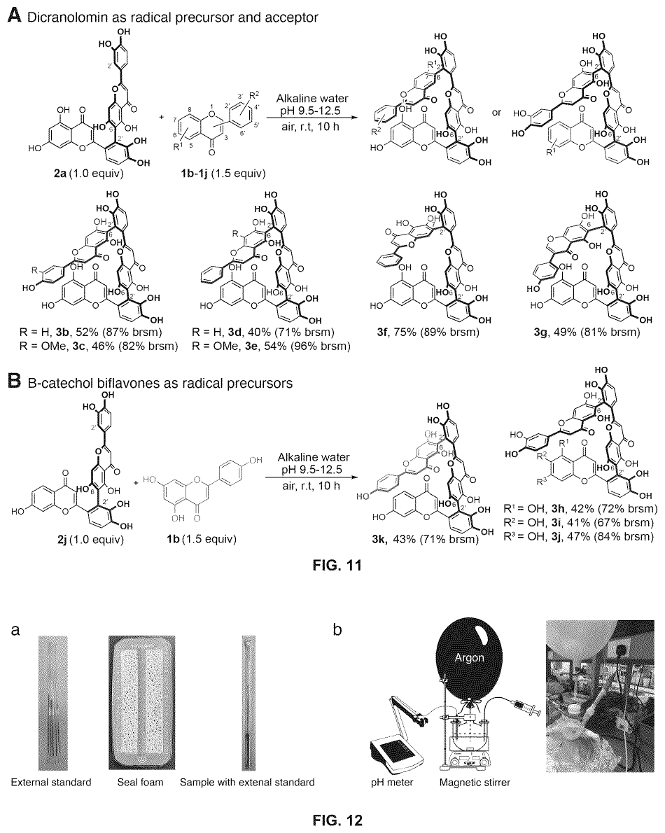

The reaction in Example 1 can be extended to the synthesis of novel triflavonoids by reacting 2a (radical precursor) with nucleophilic flavones. These triflavones share the same type of interflavonyl bonds with the general formula of Lu-(2′-6)-Lu-(2′-6)-FL (FIG. 11A). The common features for these compounds are the presence of atropisomers due to hindered rotation of the interflavonyl bonds resulting in complex ¹H and ¹³C NMR spectra. Remarkably, when 2a was reacted with trihydroxyflavones containing B-catecholic unit, it became a nucleophile and yielded products FL-(2′-6)-Lu-(2′-6)-Lu (3h, 3i, and 3j) (FIG. 11A). Biflavones other than 2a could also be a coupling partner allowing the synthesis of triflavones. For example, 2j has a catecholic unit serving as a radical precursor and it is coupled with nucleophilic apigenin to form FL-(2′-6)-Lu-(2′-6)-Ap, 3k, as the sole product (FIG. 11B). 3k is a unique triflavone containing three different monomeric flavone units. These triflavones all exist as a mixture of atropisomers that could be separated by HPLC but they isomerize overtime and gave complex ¹H NMR spectra.

### Example 10. Key Factors Influencing the Reaction Outcome

It is well-known that under alkaline conditions, flavonoids containing catechol moieties are sensitive to oxidation forming semiquinone radical intermediates (K. Kuwabara et al., Appl. Magn. Reson. 2018, 49, 911-924). However, the fates of these radicals were unclear and they are not harnessed for synthetic purposes, likely due to the formation of complex end-products. Our discovery is counter-intuitive and thus warrants an in-depth study on the key factors influencing the reaction outcome so that we can rationally maximize the yield and selectivity for synthetic use. These factors include pH, counter cations, and oxygen availability in the solution.

Determination of the pKₐ Values of Luteolin by ¹³C NMR Spectroscopy

Luteolin (200 mg) was added in the three-necks round bottle flask fitted with a pH meter under argon (FIG. 12). Degassed water (20 mL) was added with stirring. To the solution, degassed aqueous KOH solution was injected through a syringe to adjust the pH. Aliquots (500 μL) of samples at a given pH value were transferred to 5 mm NMR tubes (Armar Chemicals, Switzerland). To the same NMR tube, deuterium oxide (D20, 5.0 μL) was added introduced for deuterium lock and a DMSO-d₆ solution encapsulated in capillary tube (FIG. 12) was placed in the tube as the external standard for (at 2.50 ppm for CD₃SOCD₃, H. E. Gottlieb, V. Kotlyar & A. Nudelman, J. Org. Chem. 1997, 62, 7512-7515). The spectra were recorded and analyzed with MestReNova (Mestrelab research, version 5.2.5-4119). The resulting sigmoidal curve was subjected to nonlinear curve fitting using Prism® (GraphPad, version 5.0b and 5.0f). The dissociation constant pKₐ was calculated using the Henderson-Hasselback equation (Equation 1-4).

$$\\ \begin{matrix}{pK}_{a 1} = pH + {log}_{10} \frac{{LH}_{4}}{{LH}_{3}^{-}} & ( Equation 1 )\end{matrix}$$

$$\\ \begin{matrix}{pK}_{a 2} = pH + {log}_{10} \frac{{LH}_{3}^{-}}{{LH}_{2}^{2 -}} & ( Equation 2 )\end{matrix}$$

$$\\ \begin{matrix}{pK}_{a 3} = pH + {log}_{10} \frac{{LH}_{2}^{2 -}}{{LH}^{3 -}} & ( Equation 3 )\end{matrix}$$

$$\\ \begin{matrix}{pK}_{a 4} = pH + {log}_{10} \frac{{LH}^{3 -}}{{L}^{4 -}} & ( Equation 4 )\end{matrix}$$

Where LH₄, LH₃⁻, LH₂²⁻, LH³⁻ and L⁴⁻ are the species of luteolin in fully protonated form, first deprotonated, second deprotonated, third deprotonated 100% deprotonated form.

The chemical shift of the carbons in luteolin is dependent on the relative concentrations of the conjugate pair. For a solution at pH within pKₐ₁ range, and the species in solution is ˜100% protonated (LH₄), and the chemical shifts are that of the protonated species, δ_{low-1}. For a solution at a higher pH the species in solution is first deprotonated (LH₃⁻), and the chemical shifts are that of the deprotonated species, δ_{high-1}.

$$\\ \begin{matrix}\frac{{LH}_{4}}{{LH}_{3}^{-}} = \frac{δ - {δ}_{low - 1}}{{δ}_{high -} - δ} & ( Equation 5 )\end{matrix}$$

$$\\ \begin{matrix}\frac{{LH}_{3}^{-}}{{LH}_{2}^{2 -}} = \frac{δ - {δ}_{low - 2}}{{δ}_{high -} - δ} & ( Equation 6 )\end{matrix}$$

$$\\ \begin{matrix}\frac{{LH}_{2}^{2 -}}{{LH}^{3 -}} = \frac{δ - {δ}_{low - 3}}{{δ}_{high -} - δ} & ( Equation 7 )\end{matrix}$$

$$\\ \begin{matrix}\frac{{LH}^{3 -}}{{L}^{4 -}} = \frac{δ - {δ}_{low - 4}}{{δ}_{high - 4} - δ} & ( Equation 8 )\end{matrix}$$

Equation 7-8 were applied to determine the mole fraction of conjugate pair from chemical shift of a specific carbon. The assignment of ¹³C NMR of luteolin was further confirmed by HSQC and HMBC methods with 2D correlation with ¹H NMR.

Quantitative Measurement of Luteolin Radicals

The area under curve of luteolin radical EPR spectrum (AUC_{L}) are in linear relationship with the concentration of luteolin radicals, then concentration of luteolin radical was calculated using manganese (inside sealing putty) as reference and TEMPO (25.17 mM) as standard (Equation 9).

$$\\ \begin{matrix}\frac{{AUC}_{L}}{{AUC}_{Mn}} = \frac{{AUC}_{TEMPO}}{{AUC}_{Mn}} & ( Equation 9 )\end{matrix}$$

pH Dependent EPR Spectrum of Luteolin Radicals

Luteolin (28.6 mg, 0.1 mmol) dissolved in 10 mL KOH of different concentrations, the EPR signal of aqueous luteolin was detected immediately after transferring into EPR tube. The pH of solutions was determined after EPR measurements. Acetone and methanol were introduced to improve solubility only for samples in alkaline solution with pH<10. Numerical data were statistically analyzed by using Origin 8.0 software package (Origin Lab, Northampton, MA).

Time-Dependent EPR Spectrum of Luteolin Radicals

Luteolin (28.6 mg, 0.1 mmol) was dissolved in KOH (10 mL, 0.02 M) to give luteolin aqueous solution with pH value of 11.17. The solution was divided into tube A and tube B equally. In tube A, the solution was under stirring all the time, while in tube B, there was no stirring. The EPR signal of aqueous luteolin in both tubes was recorded immediately every 5 min after transfer into EPR tube.

Luteolin Oxidative Coupling Reaction

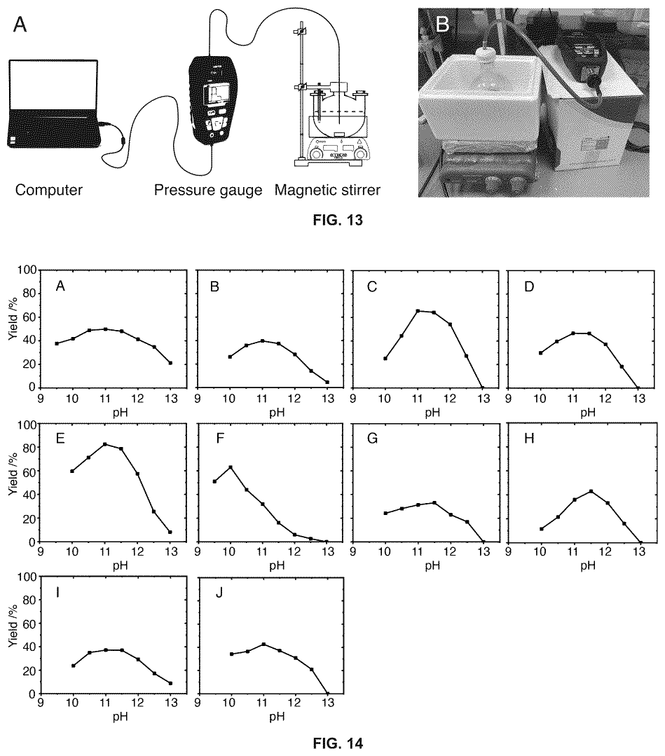

Luteolin (214.5 mg 0.75 mmol) was added into a 250 mL round bottle flask. To the flask, alkaline water (50.0 mL KOH solution 0.03 M) was introduced to dissolve the luteolin and resulted in a solution with pH of 11.78. The reaction was real-time monitored in an air-tight system as shown in FIG. 13. The pressure changes of the reaction under stirring and no stirring was recorded by a pressure gauge (NVISION, pressure recorder with vacuum range of 30 MPa, CRYSTAL Engineering Corporation).

Results and Discussion

Optimal Reaction pH

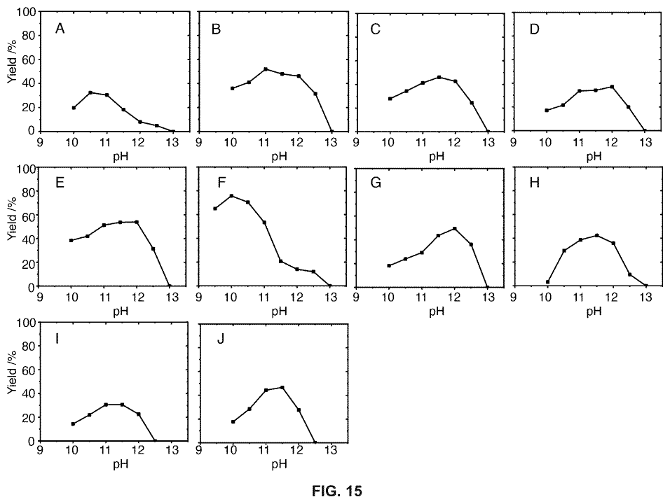

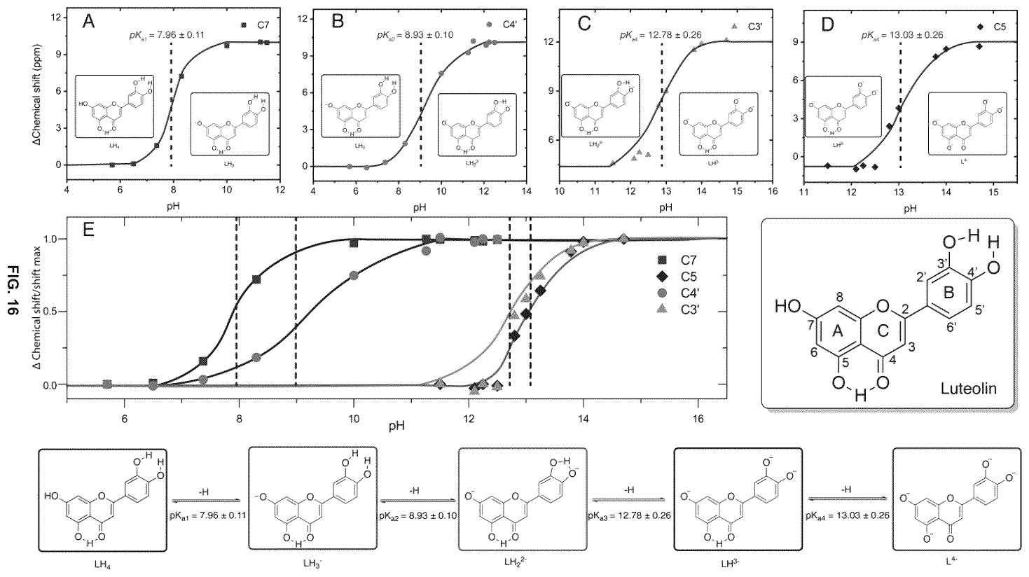

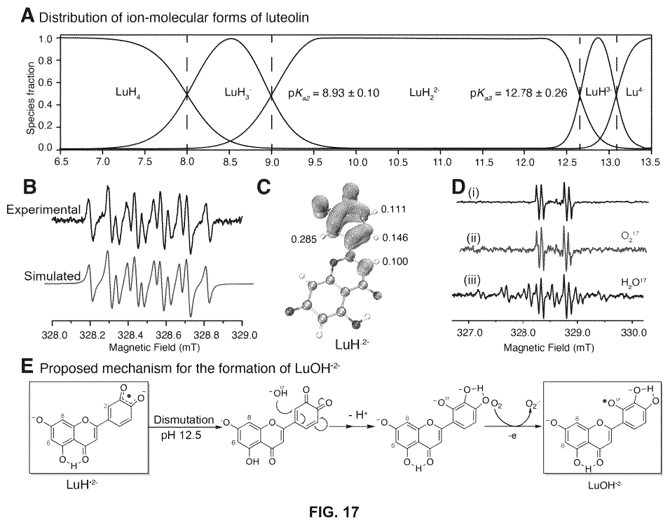

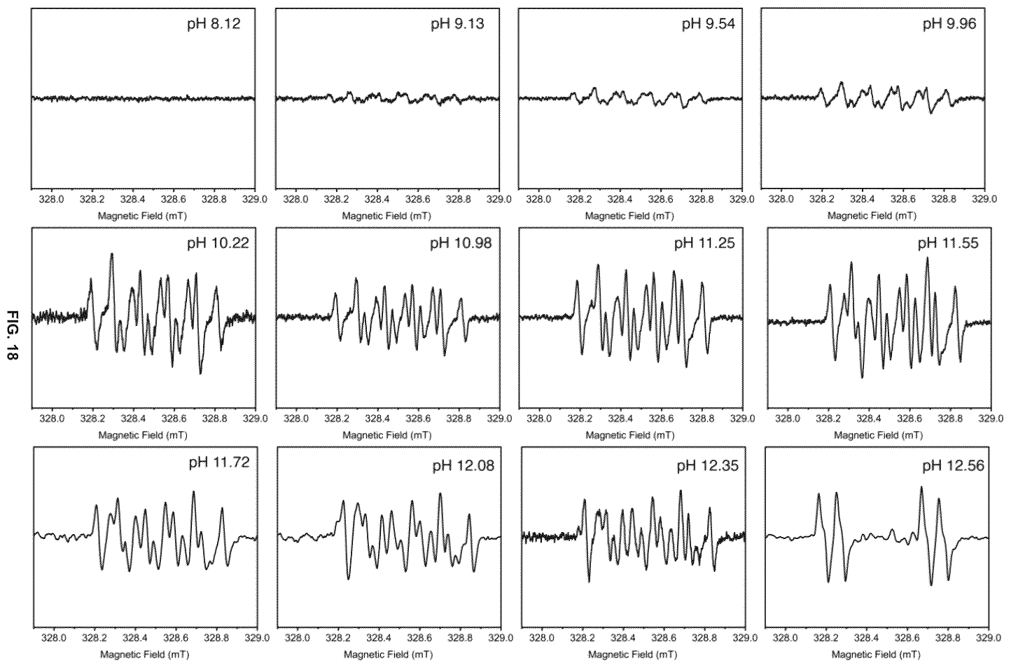

Oxidative cross-coupling of two luteolin occurred in a narrow pH range between 9.5 to 12.5, with optimal pH at 11.5 (FIG. 14), and the yield dropped quickly at pH above 13.0. For the cross-coupling reaction between two different flavones, the pH profile is dependent on individual flavones with an optimal pH of 11.0-11.5, except for 5,6-dihydroxyflavone, which has an optimal pH of 10.0 (FIG. 14B-J). This observation suggests that the optimal pH of the reaction is determined by the different pKₐ values of flavones. Similar pH profiles were found for oxidative coupling reactions between 2a and flavones (FIG. 15). The pKₐ values of the luteolin have been reported previously (G. Favaro et al., J. Fluoresc. 2007, 17, 707-714). However, the positions of deprotonations corresponding to specific pKₐ values were not unambiguously determined, and given the fact that luteolin can be oxidized at basic pH, the colorimetric pKₐ measurements are subject to interference by the luteolin oxidation products. Therefore, we determined the pKₐ values of specific phenolic protons by ¹³C NMR spectra of luteolin measured under argon (P. K. Agrawal & H.-J. Schneider, Tetrahedron Lett. 1983, 24, 177-180) (FIG. 16).

The first deprotonation occurred at C(7)-OH with pKₐ₁ of 8.00. This value is about two units larger than literature values (˜6.0) (Ŝ. Rameŝová, R. S. J. Tarebek & I. Deganoca, Electrochim. Acta 2013, 110, 646-654). Our value agrees with the observation that luteolin has poor solubility in water or slightly basic aqueous solution. The pKₐ₂ was found at 8.93 (C(4′)-OH). The pKₐ₃ and pKₐ₄ are close to each other at 12.78 (C(3′)-OH) and 13.03 (C(5)-OH), respectively (FIG. 17A). Therefore, under the optimal reaction pH, luteolin (abbreviated as LuH₄, instead of Lu to illustrate the degree of deprotonation) dianion (LuH₂²⁻) is the dominant species.

To probe the presence of luteolin radical anions, we measured the EPR spectra of air-saturated luteolin solutions in different pH and found that oxidation of LuH₂²⁻ only occurred significantly at pH above 9.5 (FIG. 18). This suggests that the oxidation of LuH₂²⁻ can only happen at pH at or greater than pKₐ of LH^{2·-} so that electron transfer-induced deprotonation can occur simultaneously:

LuH₂²⁻+O₂→LuH₂^{·-}+O₂^{·-} (1)

HO⁻+LuH₂^{·-}→LuH^{·2-}+H₂O (2)

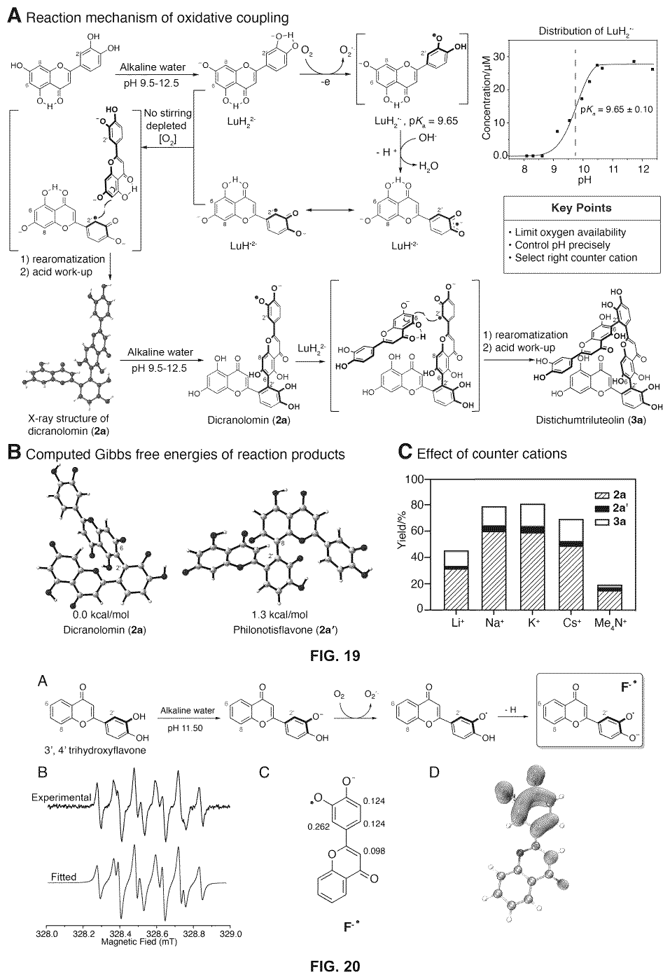

Thus, the pKₐ value of the unobserved intermediate [LuH₂-] dictates the lower limit of the pH range of the reaction. From the EPR signal intensity plots against different pH, the pKa value of LuH₂^{·-} is estimated to be 9.65 (FIG. 19A), which is close to the lower pH limit of the reaction. The LuH^{·-} radicals detected by EPR had the same hyperfine coupling patterns (FIG. 17B) that was reported in literature (Ŝ. Rameŝová, R. S. J. Tarebek & I. Deganoca, Electrochim. Acta 2013, 110, 646-654). The spin density of LuH^{·2-} map shows that C2′ has the highest density (FIG. 17C).

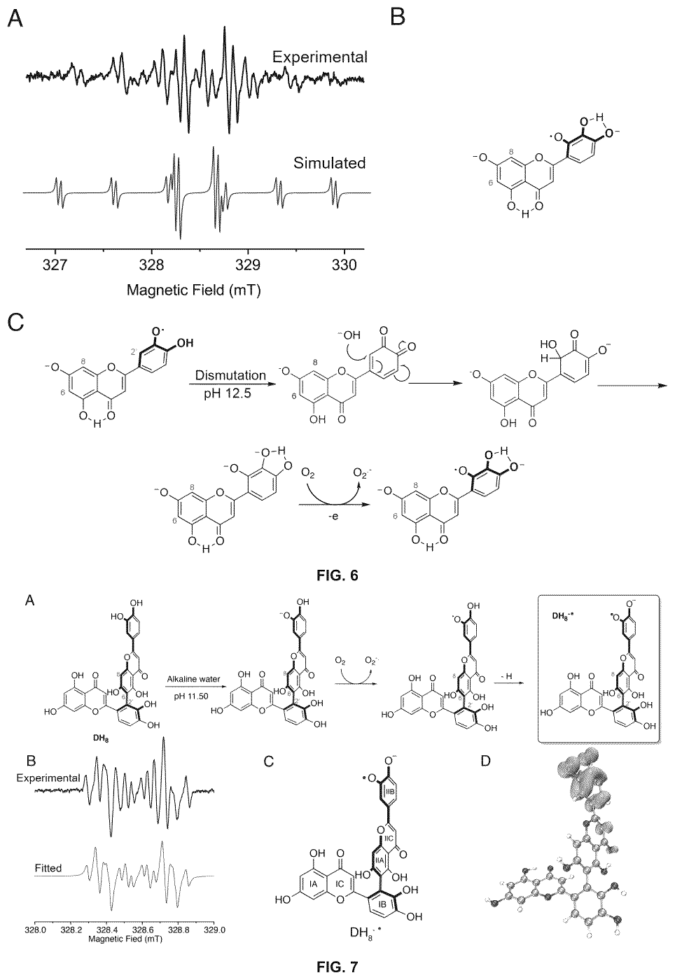

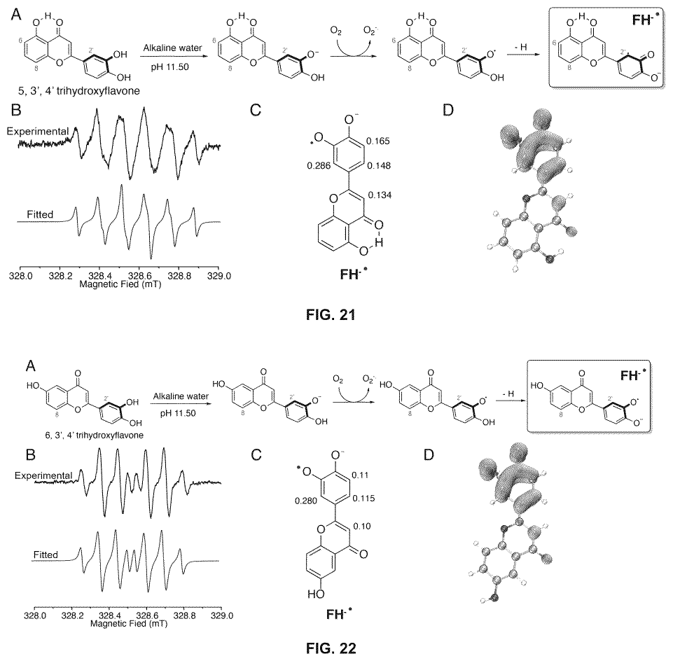

The ortho semiquinone radicals of other flavones with catecholic B rings were detected and C2's also had the highest spin density (FIGS. 4 and 20-22), in agreement with the fact that C2′ is the major site of the coupling reaction. At higher pH (>12.5), the LuH^{·2-} radical signal was depleted and a new radical species was detected featuring a doublet of doublets splitting pattern (FIG. 5). To pinpoint the nature of the new radical species, the EPR spectra was measured under ¹⁷O labelled oxygen and water, respectively. We found that ¹⁷O₂ did not alter the EPR peak splitting patterns (FIG. 17D). On the other hand, the EPR spectrum of luteolin (pH 12.5) in ¹⁷O-water (30% isotope purity) resulted in a new signal, a sextet due to the hyperfine coupling with ¹⁷O, suggesting the H¹⁷O⁻ addition to C2′ position (FIGS. 6 and 17D). We propose that LuH^{·2-} might undergo dismutation to give ortho quinone intermediate, which could react with hydroxide at high pH resulting in the observed LuOH^{·2-} radical, which is detrimental to the coupling reaction.

Based on these observations, we proposed a coupling reaction mechanism (FIG. 19A). In alkaline water, luteolin undergoes deprotonation at C(7)-OH and catecholic protons to give LuH₂²⁻, which undergoes single electron transfer to oxygen coupled by deprotonation to give LuH^{·2-}, under oxygen limiting conditions (simply without stirring), this radical anion couples with luteolin dianion, which is the dominant species under the reaction conditions. Computational results suggest that the C(6) of luteolin dianion has a higher electron density and thus is a preferred reaction site resulting in 2′-6 biflavone as the major product. Furthermore, our computational results also show that 2′-6 isomer dicranolomin (2a) is more stable than 2-8 isomer philonotisflavone (2a′) by 1.3 kcal/mol (FIG. 19C). Dicranolomin (2a) has two catecholic moieties. Remarkably, its reaction products with flavone monomers are highly regiospecific on the unreacted catecholic moiety (IIB), suggesting that semiquinone radicals from IIB shall be involved (FIG. 19A). The EPR spectrum of ortho-semiquinone radical of 2a (FIG. 7) shows complicated signals due to multiple radical species, including the semiquinone formed on IIB with aH1 of 0.285 mT (Table 15). The other radical may reside in the IB ring with aH1 of 0.43 mT (Table 15). The semiquinone radical at IB ring did not participate in coupling reaction as linear trimer Lu-Lu-FL, instead branched trimer, was observed and isolated.

TABLE 15
Hyperfine coupling constants (aHn) of the protons, linewidth (LW) and
center field (CF) for flavonoid radicals. The spectra were simulated
by JEOL IsoSimu/Fa Version 2.2.0 isotropic simulation program.

| Name | pH | G value | a_{H1} | a_{H2} | a_{H3} | a_{H4} | LW/mT | CF/mT |
|---|---|---|---|---|---|---|---|---|
| 3′,4′-dihydroxyflavone | 10 | 2.0002 | 0.262 | 0.124 | 0.124 | 0.098 | 0.02 | 328.348 |
| 5,3′,4′-trihydroxyflavone | 11 | 2.0002 | 0.286 | 0.165 | 0.148 | 0.134 | 0.02 | 328.348 |
| 6,3′,4′-trihydroxyflavone | 11 | 2.0002 | 0.280 | 0.115 | 0.110 | 0.100 | 0.02 | 328.348 |
| 7,3′,4′-trihydroxyflavone | 11 | 2.0002 | 0.286 | 0.132 | 0.105 | 1.100 | 0.02 | 328.348 |
| LH²⁻• | 11.5 | 2.0002 | 0.285 | 0.146 | 0.111 | 0.100 | 0.02 | 328.348 |
| [LH²⁻ + O₂]• | 11.5 | 2.00025 | 0.43 | 0.05 | | | | 328.350 |
| LH²⁻• | 12.5 | 2.0004 | 0.505 | 0.084 | 0.1 | | | |
| Dicranolomin | 11.5 | 2.0002 | 0.285 | 0.146 | 0.111 | 0.100 | 0.02 | 328.348 |
| | | 2.00025 | 0.43 | 0.07 | 0.015 | | | |

Impact of Counter Cations in the Coupling Reaction

For two dianions (LuH^{·2-} and LuH₂²⁻) to react, the charge repulsions have to be overcome possibly by ion pairing with countercation. Therefore, we examined the effects of different counter cations on the reaction and we found that tetramethylammonium (Me₄N⁺, added as Me₄NOH) gave the lowest yield (<20%). Lithium performed better but not as good as cesium, sodium and potassium (˜80%) (FIG. 19D). These results suggest that the counter-cations not only offset the anionic charges but might also facilitate the reaction through bridging both coupling partners closer to each other with weak coordination interactions. In this regard, small lithium ion is not as effective as larger alkali metal ions. In aqueous solution, alkali metal ions shall be present as hydrates and the coordination bonds with phenolates of the luteolin dianions shall be fairly weak and dynamic.

Impact of Oxygen Availability

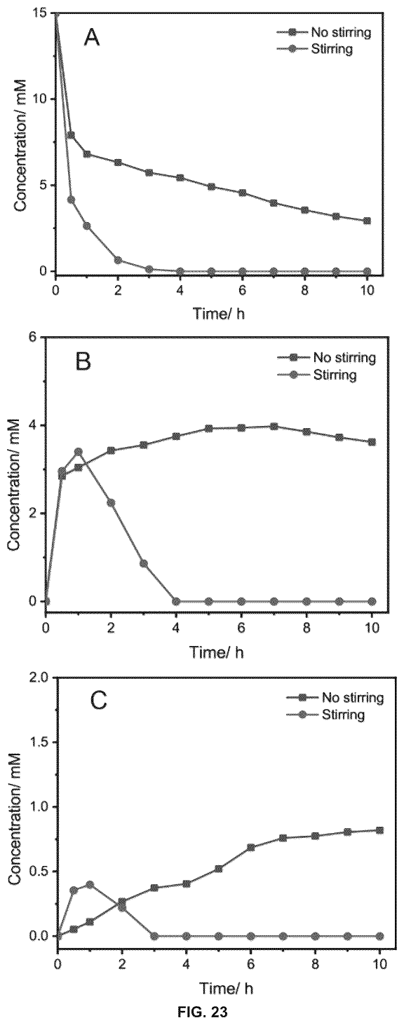

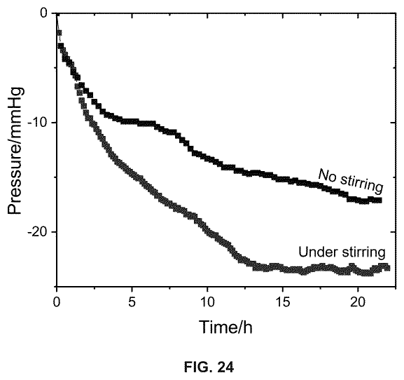

Although ortho-semiquinone radical of luteolin has been observed previously, the end-products were found to be complex not characterized (Ŝ. Rameŝová, R. S. J. Tarebek & I. Deganoca, Electrochim. Acta 2013, 110, 646-654), likely due to overoxidation by excessive oxygen in the solution. The positive outcome of our case is likely due to limiting oxygen and by conducting the reaction unstirred, which is counter-intuitive. We compared the reaction dynamics of alkaline luteolin solution in two test tubes; one tube was magnetically stirred vigorously (so that oxygen is in excess supply) while the other tube was not stirred (oxygen availability is dependent on the diffusion of the gas phase oxygen into solution). The coupled products in the stirred tube could not be detected after 4 hours, while in the unstirred tube, both 2a and 3a showed two major products after 10 hours (FIG. 23). The air saturated water has a dissolved oxygen concentration of about 256 μM and its concentration is lower in alkaline water (W. Xing et al., (2014). Oxygen solubility, diffusion coefficient, and solution viscosity. In W. Xing, G. Yin & J. Zhang (Eds.), Rotating Electrode Methods and Oxygen Reduction Electrocatalysts (pp. 1-31). Elsevier). At the beginning of the reaction, the dissolved oxygen in both tubes was quickly depleted by reacting with LH₂²⁻. However, stirring replenishes the dissolved oxygen, which undergoes radical coupling reaction with LH^{·2-} leading to overoxidation. While in the unstirred tube, such a reaction is prevented due to depleted oxygen and the slow diffusion of the gaseous oxygen to the undisturbed solution which will prevent product formation over time (FIGS. 13 and 24). Therefore, limiting oxygen availability is a key factor for oxygen mediated oxidative coupling reaction of flavones.

### Example 11. Contrasting Bioactivity of the Flavonoids Dimers and Trimers

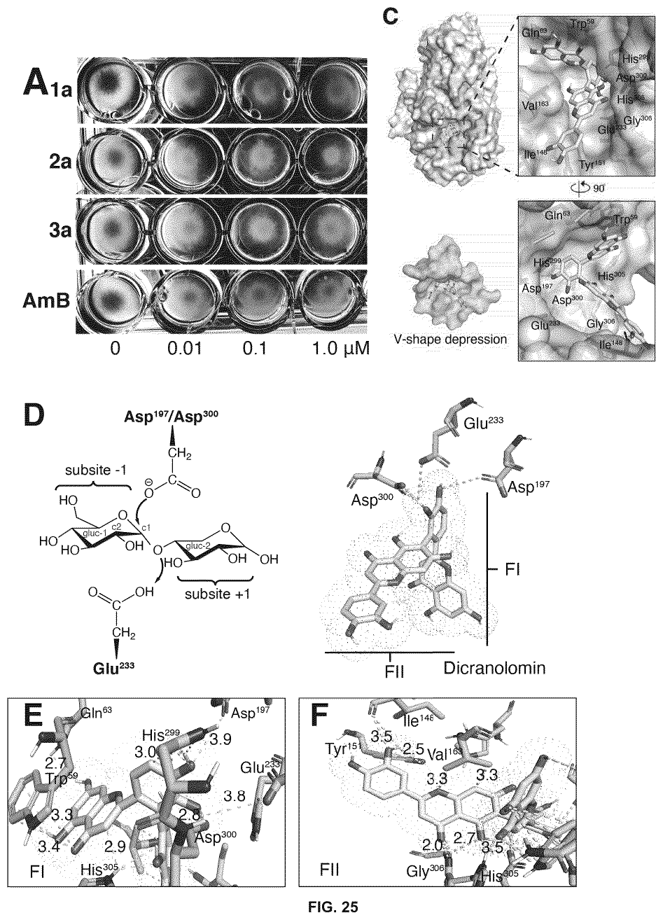

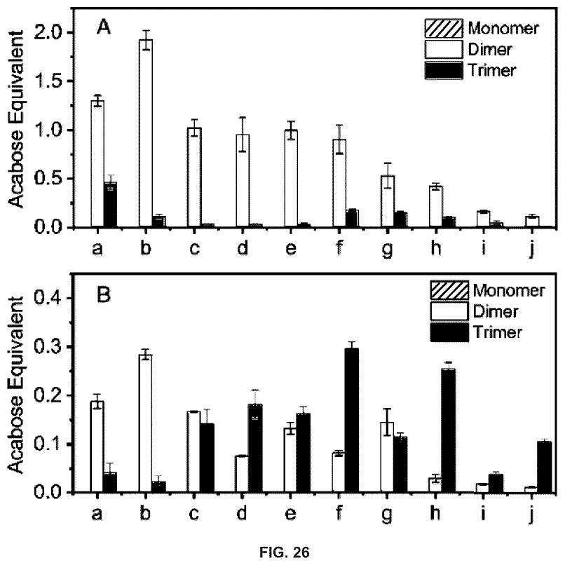

It was suggested that, moss utilizes a large amount of bioresource in the synthesis of luteolin dimers and trimers, because of the need to defend against the microbial stress endured by the moss while growing on rotting wood in wet forests (H. Geiger et al., J. Hattori Bot. Lab. 1997, 83, 273-308). To test this hypothesis, we measured the antifungal activity of luteolin, 2a, and 3a using Aspergillus niger as a model fungus (FIGS. 25 and 26).

Antifungal Activity Study

The antifungal activity was evaluated against two fungi, Aspergillus niger ATCC 16888 and Botrytis cinerea ATCC 11542, isolated and identified with ITS gene (GenBank accession number AY373852, A. S. Taha et al., BioResources 2019, 14, 6025-6046). The bioassay was evaluated using the radial growth technique method (P. Wayne, Reference method for broth dilution antifungal susceptibility testing of yeasts, approved standard. CLSI document M27-A2, 2002). Both fungal strains were cultivated aerobically on PDA medium at 25° C. for 14 days. The fungal spores were collected with sterile cotton swabs and suspended in sterile water, and the concentrations were adjusted according to the requirements of the antifungal assays to be performed.

Determination of In Vitro Antifungal Activity

Flavonoids oligomers were dissolved in DMSO (1.0 mg/mL), then transferred into sterilized warm PDA medium (40 to 45° C.) to achieve a final concentration of 1.0 mg/mL, 0.6 mg/mL and 0.4 mg/mL, before immediately pouring into 24-well sterile plastic microtitration plates containing flat-bottomed wells (Corning Incorporated, costar). After the plates were cooled to room temperature, 10 μL of freshly made A. niger suspension (1.25×10⁻⁶/mL) was inoculated onto the agar of each well. Drug-free agar with 1% DMSO was used as the negative control and amphotericin B was used as the positive control.

From a 7-day-old colony, the fungus with discs of 9 mm diameter was transferred to the center of the treated PDA plates and controls. All the plates were incubated at 26±1° C. for 7 days. All the tested concentrations, as well as positive and negative controls, were measured in triplicate.

High-Throughput Assay of Starch Hydrolase Inhibition Activity

The inhibitory activity of each fraction obtained on α-amylase and α-glucosidase were determined using the turbidity measurement according to a reported method (Liu, Song, Wang, & Huang, 2011, Journal of agricultural and food chemistry 2011 Vol. 59 Issue 18 Pages 9756-9762). Corn starch (20 mg/mL) was suspended in sodium phosphate buffer (0.1 M, pH 6.9) and gelatinized at 100° C. for 2.5 min. The inhibitor solution was diluted in buffer to appropriate concentrations. Acarbose was used as a positive control and a reference standard. α-amylase solution (2 U/mL in buffer, 20 μL) or α-glucosidase solution (1×10−2 U/mL in buffer) was pre-incubated with inhibitor solution (20 μL) with series of concentrations in a 96-well microplate and kept at 37° C. for 15 min. The reaction was initiated by injecting 60 μL of the gelatinized corn starch solution. The turbidity changes were recorded at 660 nm every minute for 2 h using a Synergy HT microplate reader (Biotek Instruments Inc., Winooski, VT, USA). The inhibition percentage was calculated using Equation 10.

Inhibition (%)=(AUCₛₐₘₚₗₑ−AUC_{control})/AUCₛₐₘₚₗₑ×100% (Equation 10)

Where AUCₛₐₘₚₗₑ is the area under the inhibitory curve and AUC_{control} is the area under the curve negative control. The IC₅₀ can be defined as the concentration of an inhibitor that produces 50% inhibition of enzyme activity under a specified assay condition. It was obtained from interpolation of percentage of inhibition against inhibitor concentration curve. In order to avoid run-to-run error due to fluctuation of enzymatic activity, the acarbose equivalent (AE) was used to express the inhibitory activity of sample based on the following equation:

AE of a sample=IC₅₀ of acarbose/IC₅₀ of a sample (Equation 11)

Molecular Docking

Ligand preparation: All ligands including acarbose, luteolin, 2a, 2b and 3a were selected as the ligands and virtually constructed through their crystal structures. These ligand molecules were drawn and saved as 3D conformers in .cif format. The structure of these ligands was then converted into .pdb format via PyMol. Subsequently, the ligand molecules were uploaded as an input file using .pdb format onto Autodock, followed by output as .pdbqt format file.

Preparation of protein molecule: The active center of a mammalian alpha-amylase, 1ppi, was selected and retrieved in a .pdb format file from RCSB (M. Qian et al., Biochemistry 1994, 33, 6284-6294). The target amylase was loaded on the graphical user interface of Autodock (G. M. Morris et al., J. Comput. Chem. 1998, 19, 1639-1662; and R. Huey et al., J. Comput. Chem. 2007, 28, 1145-1152) in .pdb format. The amylase was prepared for docking by removal of the acarbose molecule, deleting water molecules, adding polar hydrogen atoms and adding Kollman charges to the macromolecule. Thereafter, the amylase was converted from a .pdb format to a .pdbqt format file. A grid box was selected and adjusted to specific dimensions of the docking site. The output file of the grid dimensions was saved as a .txt file.

Docking through AutoDock Vina and visualization using PyMOL: AutoDock Vina (O. Trott & A. J. Olson, J. Comput. Chem. 2010, 31, 455-461) is a virtual screening technique to predict the optimal bound conformations of ligands to a target protein of known structure. To conduct Autodock, the prepared ligands and amylase were used in .pdbqt format and a configuration file was set up in a .txt file. Docking through AutoDock Vina was executed using command prompt and the results of the docking were analysed through PyMOL. PyMOL is a molecular visualization program widely used for three-dimensional (3D) visualization of proteins and small molecules. The output .pdbqt file from AutoDock vina and amylase in .pdbqt format was loaded on the graphical interface of PyMOL. There, the docked structure was visualized under the “molecular surface” and “cartoon” option. The active site of the docking was shown using the “pockets” function of PyMOL.

Results and Discussion

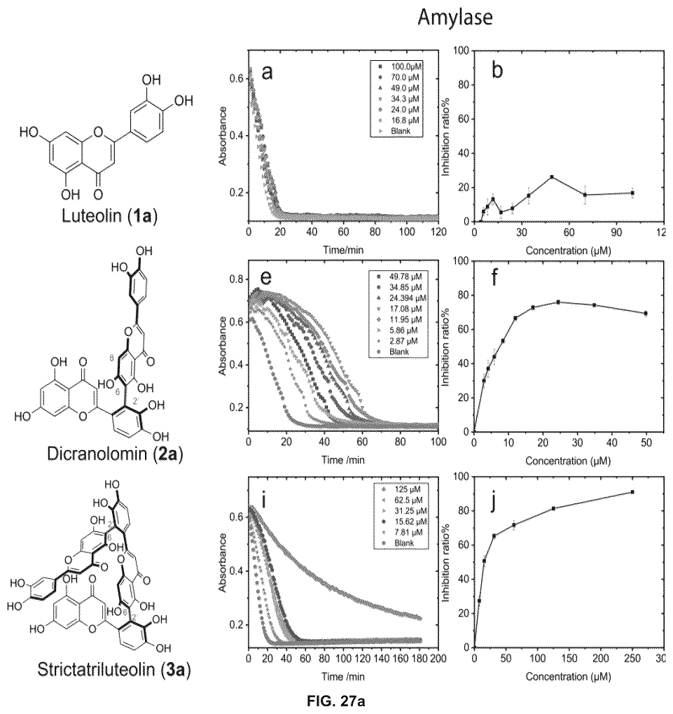

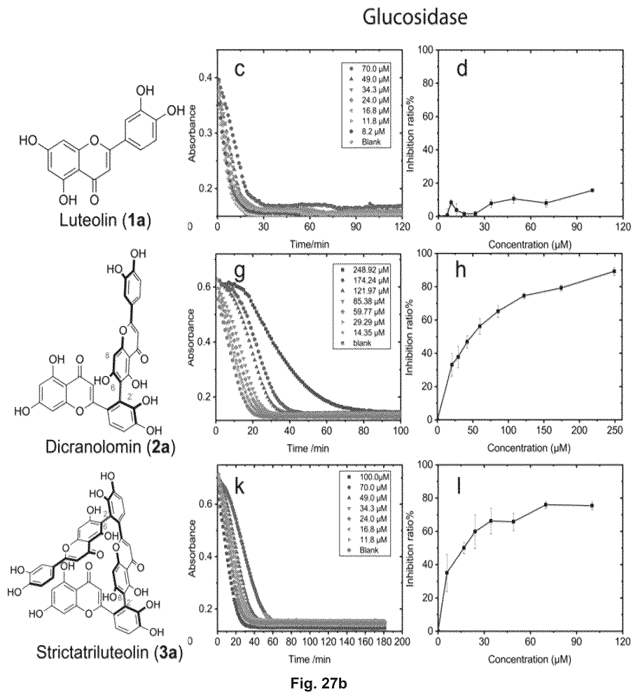

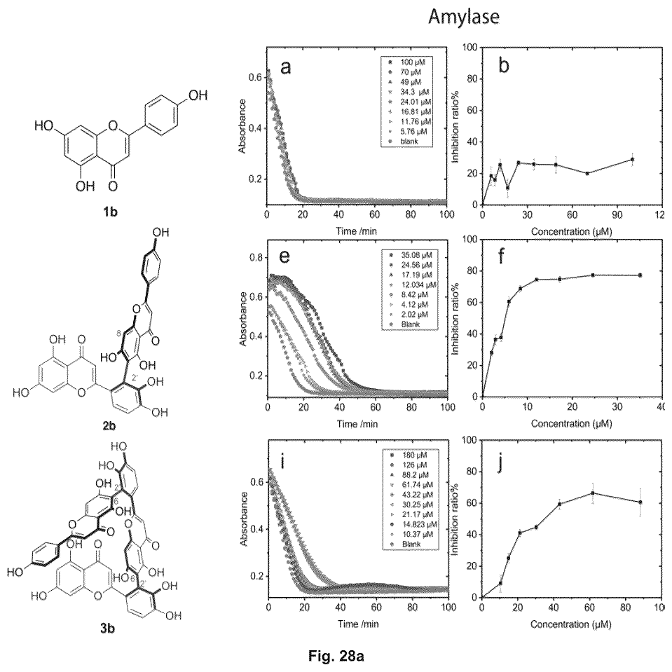

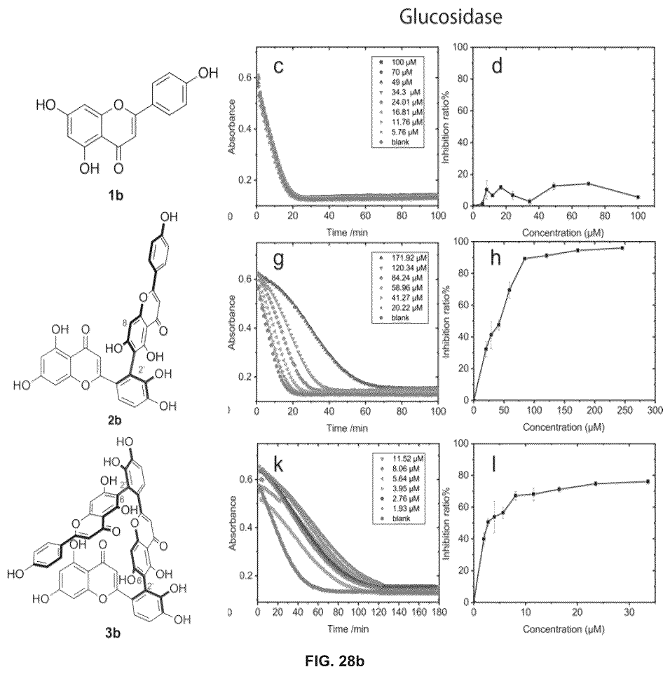

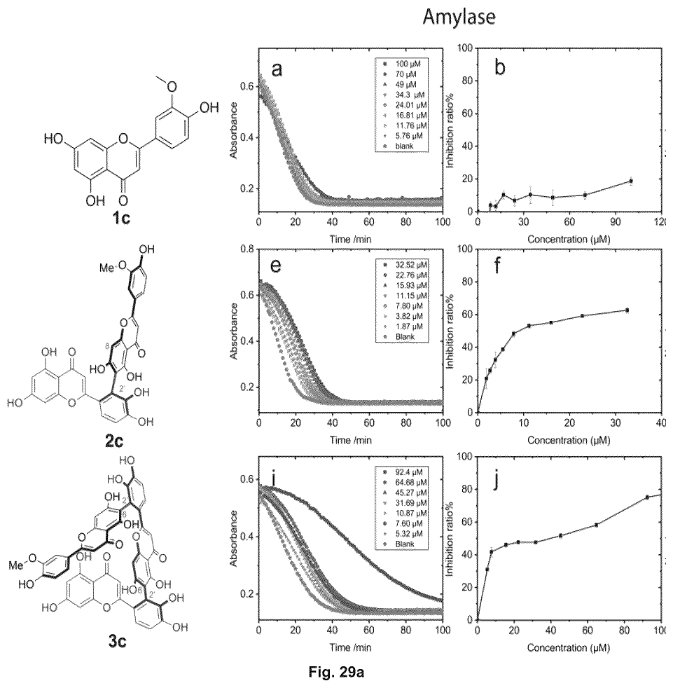

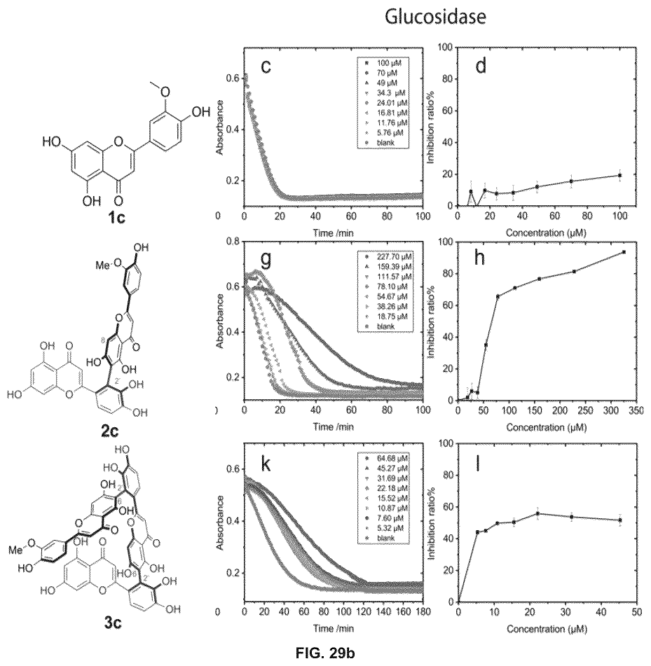

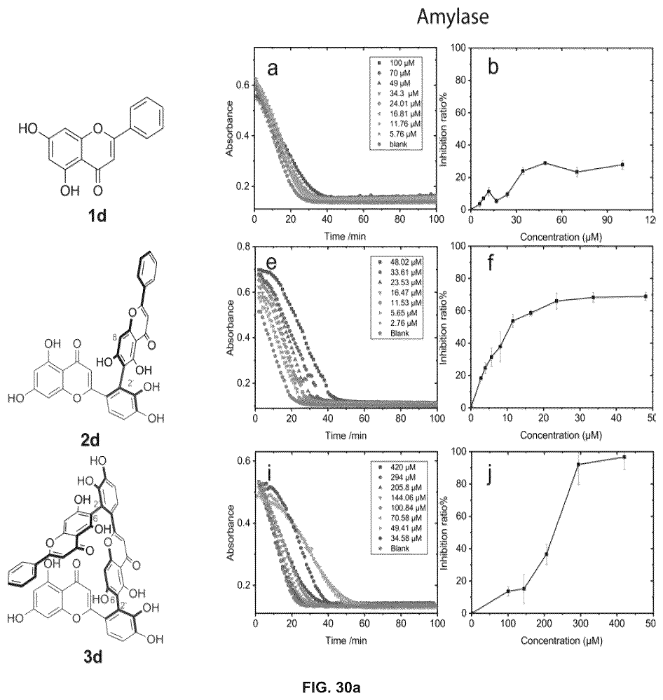

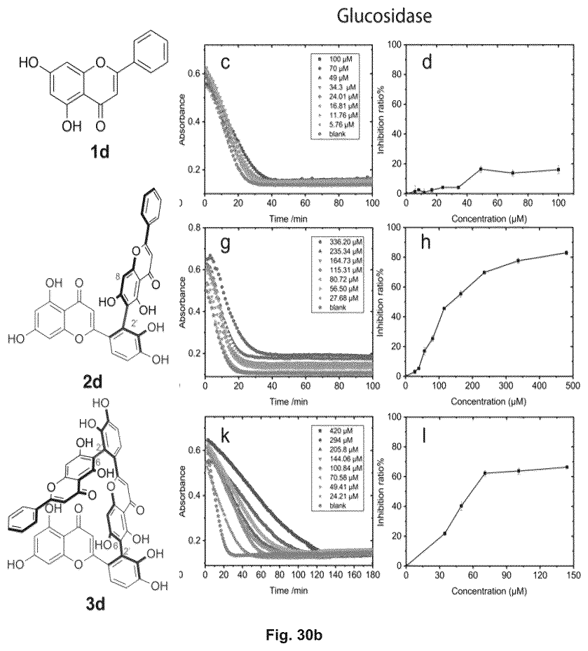

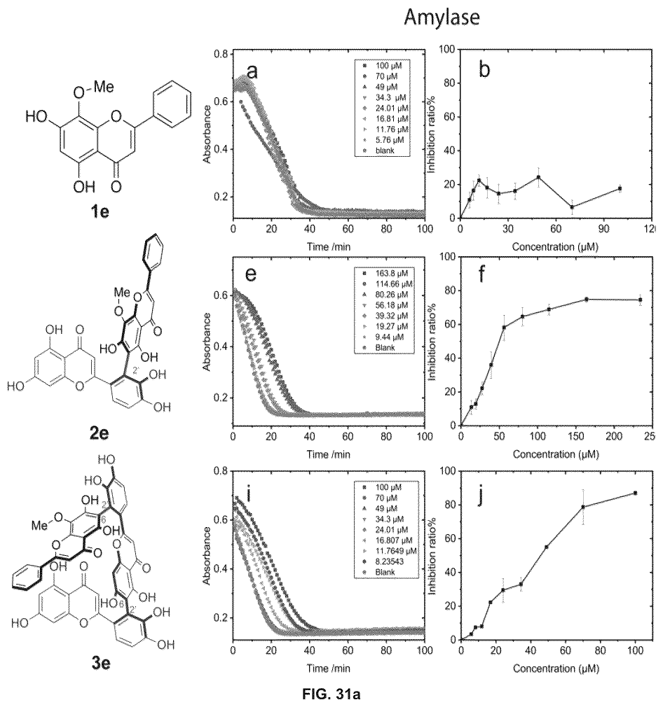

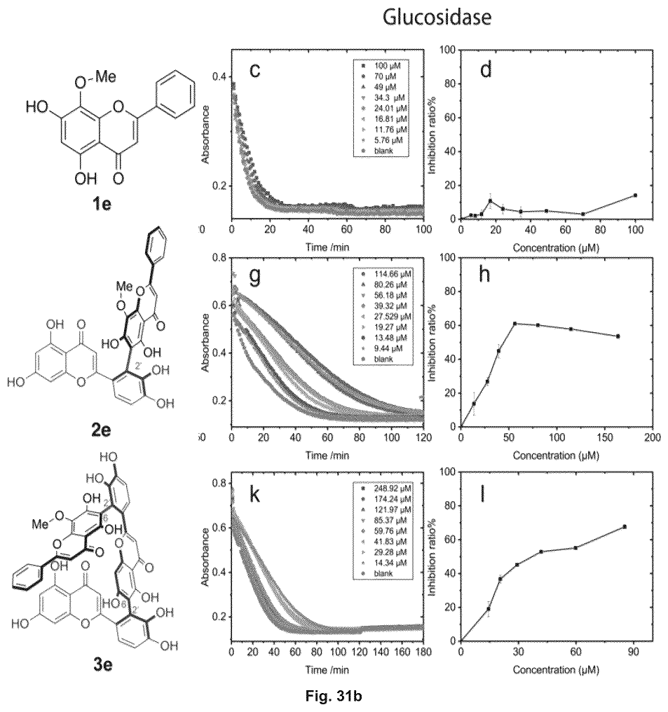

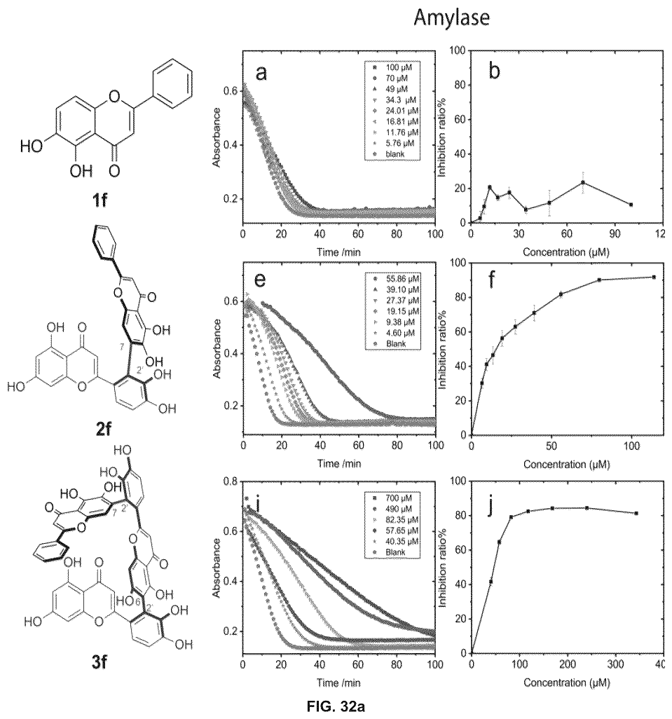

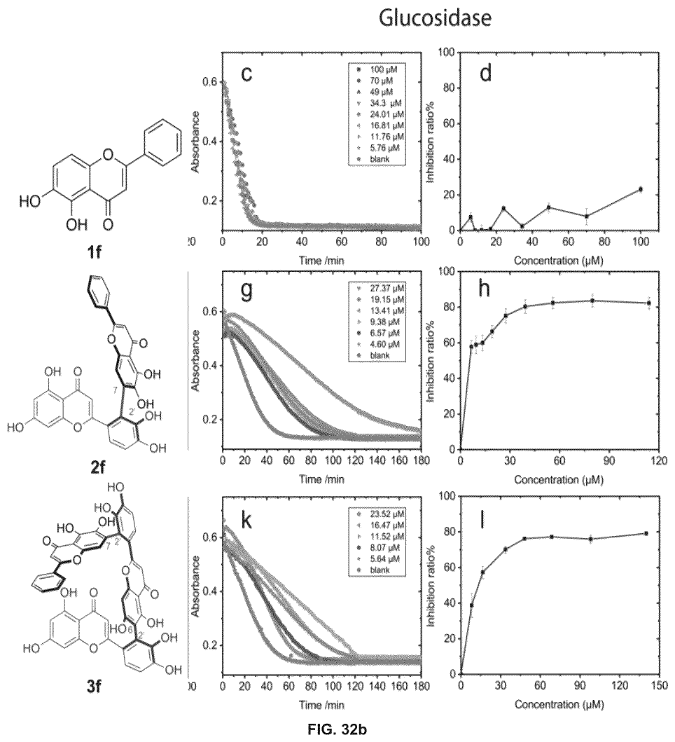

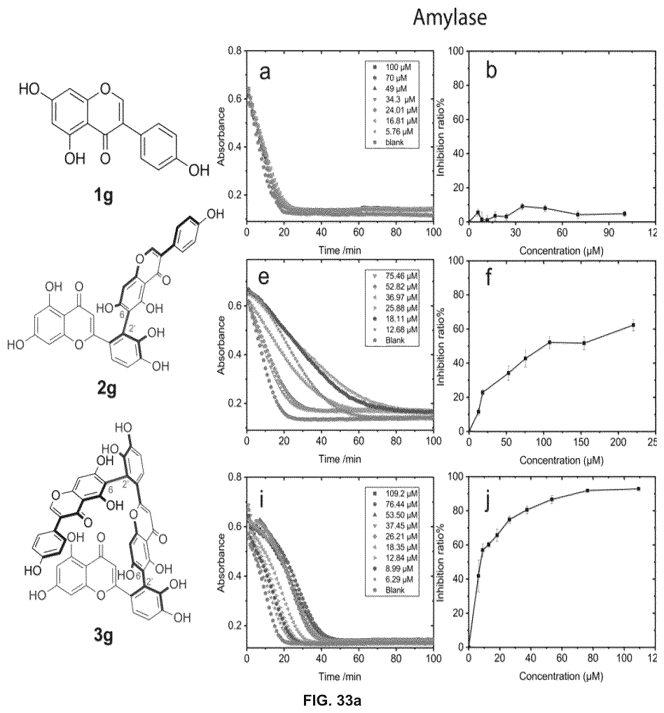

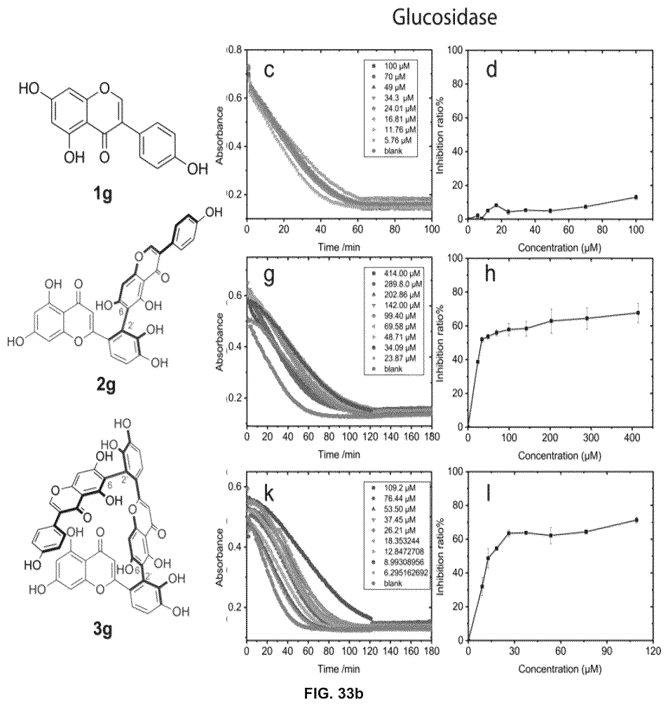

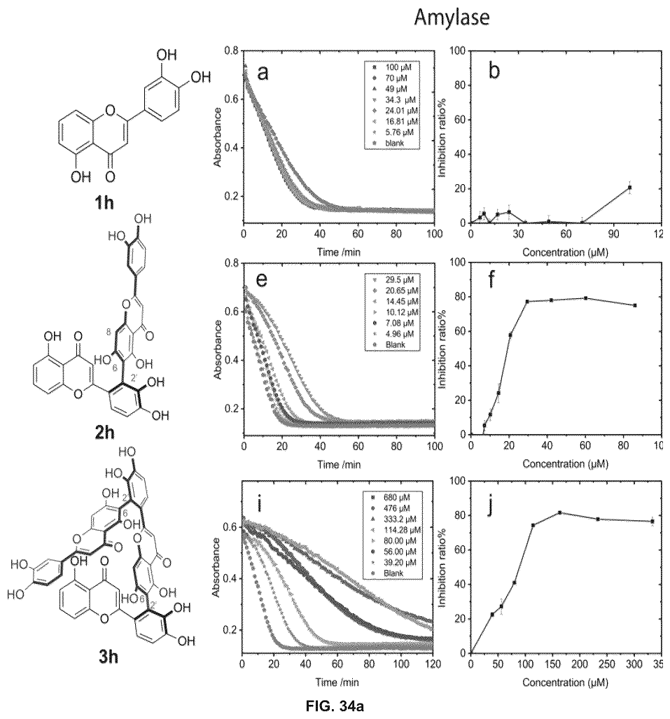

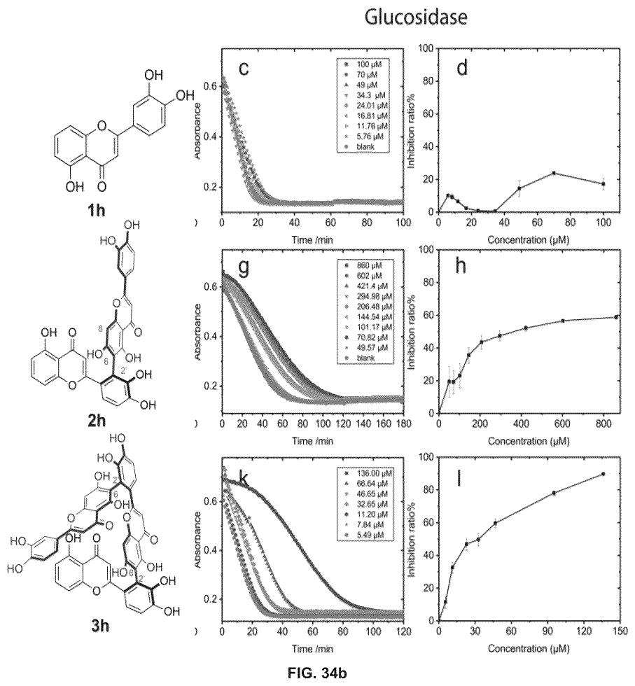

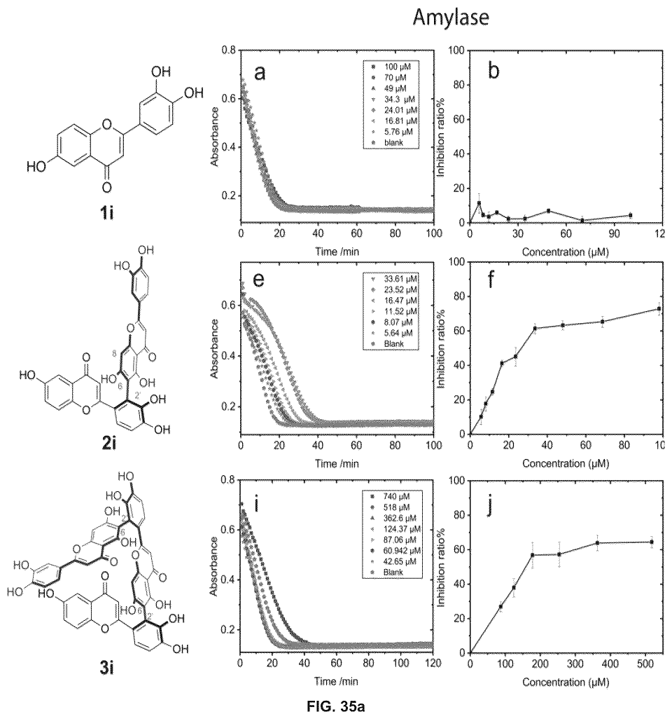

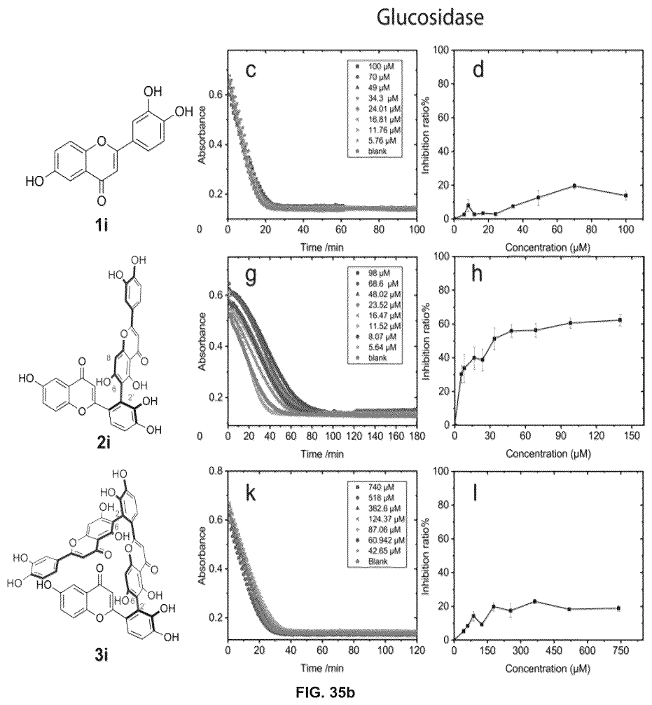

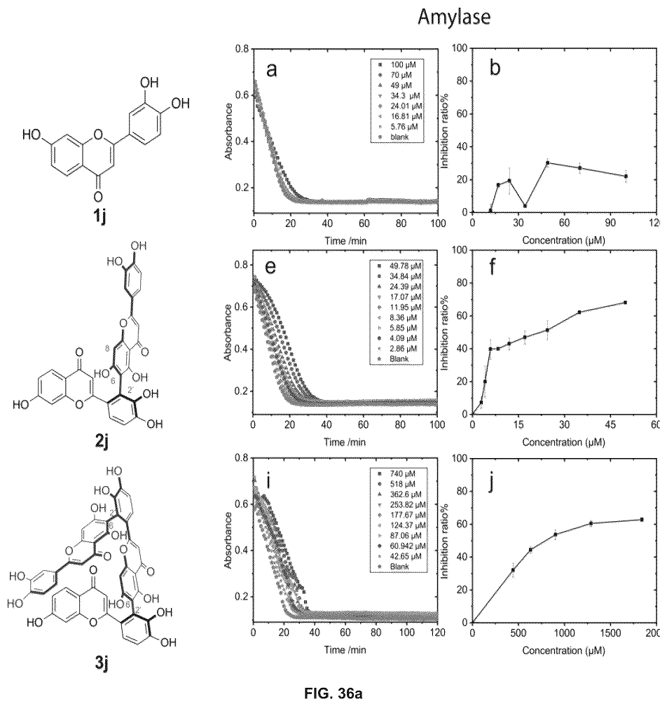

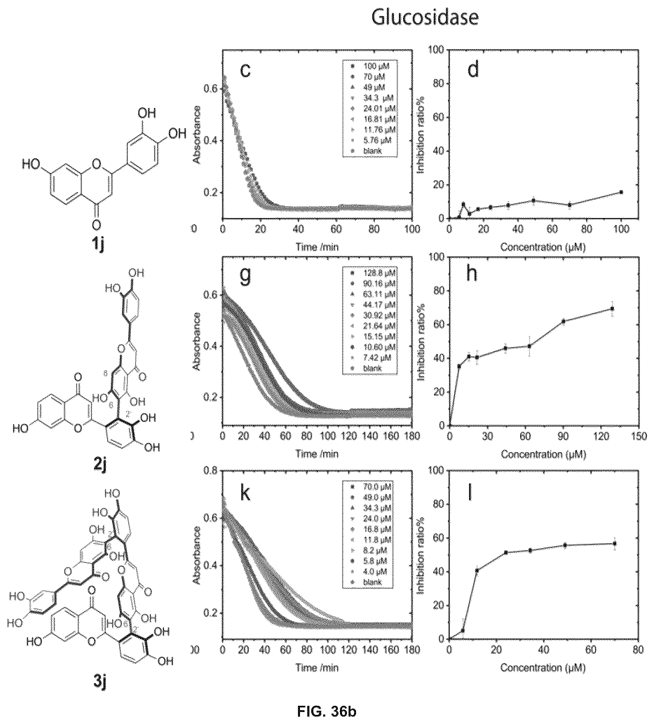

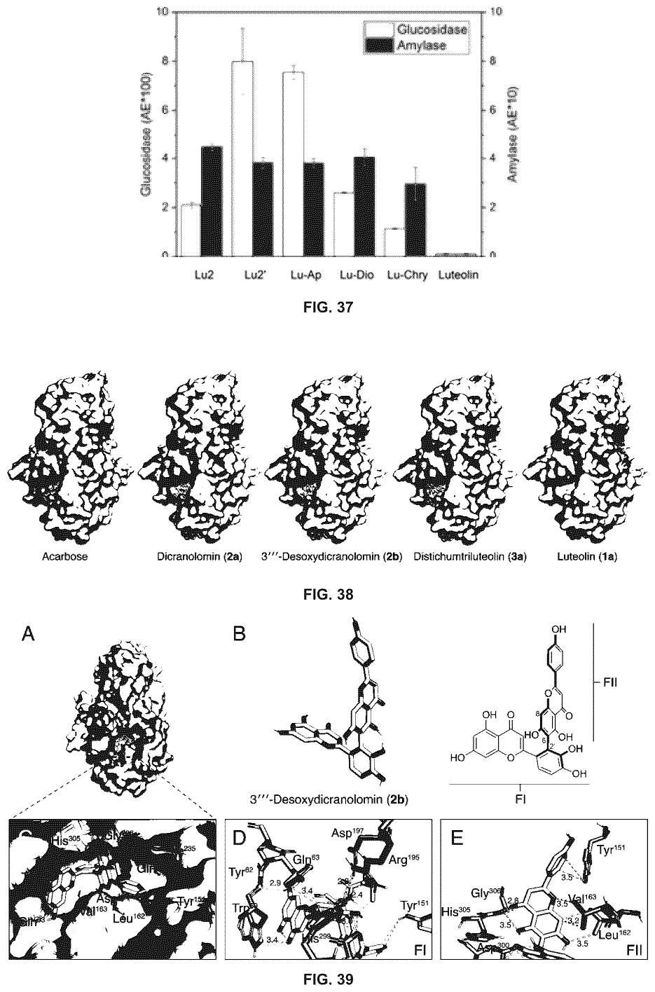

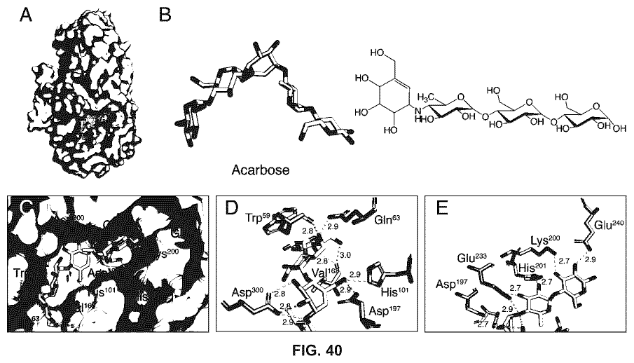

2a and 3a could inhibit the growth of A. niger with IC₅₀ of 0.86 μM and 0.96 μM, respectively, which is comparable to that of amphotericin B (IC₅₀ of 0.50) in a dose-dependent manner. Notably, dimer 2a shows slightly higher activity than trimer (3a). Plant flavonoids protect the plant from being eaten by insects by inhibiting digestive enzymes such as alpha-amylase and alpha-glucosidase. We measured the activity of selected flavone dimers and trimer (2a-2f, 3-3f) in inhibiting α-amylase and α-glucosidase (FIG. 26-37) and found that dimers 2a and 2b show comparable (2a) or even higher (2b) activity than acarbose, an antidiabetic drug. Molecular docking by using the crystal structure of a pancreatic α-amylase with acarbose complex (M. Qian et al., Biochemistry 1994, 33, 6284-6294) found that flavone oligomers (2a, 2b, 3a) are located at the active center while that of luteolin is not, which might explain why luteolin has no activity. The active site region of α-amylase is a V-shaped depression located at the carboxyl end of Glu233, Asp300, and Asp197. The molecular shape of 2a and 2b (FIG. 38-39) happens to be V-shaped (similar to that of acarbose in FIG. 40) and fits nicely in depression with one B-ring near the catalytic active groups and form hydrogen bonding through the catecholic group with Glu233, Asp300, and Asp197.

### Example 12. In Vivo Studies

A 10% (w/v) starch solution was prepared by addition of distilled water to reagent grade corn starch and heating the solution it in a boiling water bath for about 15 minutes until it was completely gelatinized.

8-week-old male wild-type C57BL/6J mice were fasted overnight (free water intake), and weighed the next day. The tail of the mice was trimmed and the blood was taken for measurement of fasting blood glucose concentration with a Roche blood glucose meter.

Feeding method a, “YX2+Starch”: Luteolin dimer (2a) was gavaged as a 2 mg/mL water solution, at the dosage level of 20 mg/kg body weight of the animal. Thirty minutes later, the 10% (w/v) starch solution was gavaged at 1 g/kg body weight.

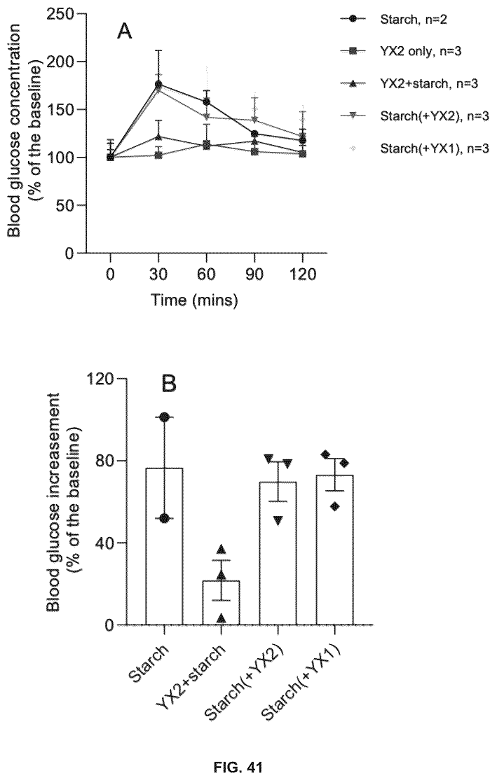

Feeding method b, “Starch (+YX2)”: Luteolin dimer (2a) was mixed with starch at 0.2% ratio by dried weight and mixed with water (1:9 by weight) to give a slurry labelled as Starch (+YX2) (FIG. 41). The mixture was gavaged to mice at with 1 g/kg body weight. In comparison, luteolin (1a or YX1) was mixed with starch at 0.2% by weight. The resulting mixture was further mixed with water to give 10% starch slurry labelled as starch (+YX1) (FIG. 41). The mice were gavaged with 1 g/kg body weight.

The blood glucose values were measured at 30, 60, 90, 120 minutes to make the blood glucose curve, GraphPad draws the blood glucose response curve after meal, and the area under the curve was calculated.

Results and Discussion

The glycemic response of the mice fed with different samples is shown in FIG. 41 in which YX2 refers to luetolin dimer (2a) and YX1 refers to luteolin monomer (1a).

From the results (FIG. 41A), it clearly shown that feeding the mice with starch (1 g/kg body weight by gavage resulted in blood glucose increase to peak value after 30 minutes and the values gradually dropped up to 120 minutes.

Feeding the mice only with luteolin dimer (YX2) at 20 mg/kg body weight did not lead to glycemic response.

If the YX2 was fed at 20 mg/kg dose 30 minutes before administration of starch at 1 g/kg body weight), there was no glycemic response (curve YX2+starch). In contrast, if the mice were fed with 0.2% mixture of YX2 in starch, the glycemic response was comparable to that of the mice fed only with same dose of starch (1 g/kg body weight) (curve: starch (+YX2).

Finally, if the mice were fed with 0.2% mixture of luteolin with starch, the glycemic response curve was almost the same as that of the mice fed only with same dosage of starch. This results suggested that for luteolin dimer 2a to be effect in suppressing glycemic response after consumption of starch, 2a needs to be taken 30 minutes before meal.

1. A method for coupling a flavonoid-containing compound, said method comprising:
(i) providing one or more flavonoid-containing compounds; and
(ii) contacting the one or more flavonoid-containing compounds with a base in the presence of air;
wherein the base is selected from the group consisting of a metal carbonate, a metal hydroxide and a base of the formula R₄NOH, where each R independently represents H or C₁₋₄ alkyl; and
step (ii) is performed in a sealed reaction vessel comprising air and a reaction mixture comprising the one or more flavonoid-containing compounds, the base and water, where the air occupies from 10 to 95% of the volume of the reaction vessel at 25° C. and a pressure of 101 kPa.

2. The method according to claim 1, wherein the base is selected from one or more of the group consisting of lithium hydroxide, sodium hydroxide, potassium hydroxide, caesium hydroxide, lithium carbonate, sodium carbonate, potassium carbonate, and alkaline water.

3. The method according to claim 2, wherein the base is selected from one or more of the group consisting of sodium hydroxide and potassium hydroxide.

4. The method according to claim 3, wherein the base is potassium hydroxide.

5. The method according to claim 1, wherein the pH of the reaction mixture in step (ii) is from about 10 to about 14.

6. The method according to claim 1, wherein the temperature of the reaction mixture in step (ii) is from 15 to 30° C.

7. The method according to claim 1, wherein step (ii) is performed for a time of from about 1 to about 20 hours.

8. The method according to claim 1, wherein step (ii) is performed without stirring.

9. The method according to claim 1, wherein the flavonoid-containing compound comprises a backbone selected from the group consisting of a flavone backbone, an isoflavan backbone, a neoflavonoid backbone, a flavan backbone, a flavanone backbone, an isoflavanone backbone and an isoflavone backbone, which backbone is optionally substituted with one or more substituent selected from the group consisting of hydroxyl, methoxy, glycosyl, alkoxy, NO₂, F, CN, SH, CF₃, Cl, Br, I, ═O, ═CH₂, C₁₋₁₈ alkyl, C₁₋₁₈ fluoroalkyl, —O—C(O)—R (where R represents a C₁₋₆ alkyl group), C₂₋₁₈ alkenyl, prenyl, phytyl, exocyclic C₃₋₆ cycloalkyl, exocyclic C₅₋₆ cycloalkenyl, phenyl, phenoxyl, C₁₋₁₈ N-aklylaminyl, and C₁₋₁₈ N,N-dialkylaminyl, 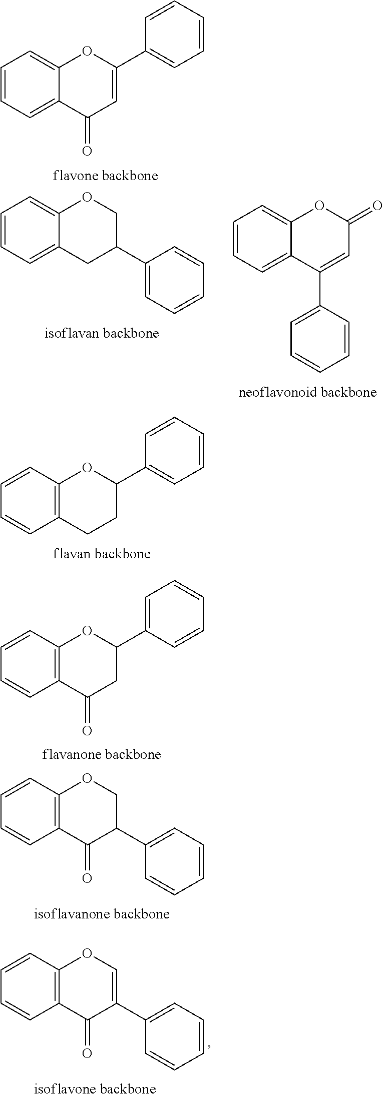
or wherein the flavonoid-containing compound comprises a dimer or trimer thereof.

10. The method according to claim 1, wherein the flavonoid-containing compound comprises a flavone backbone.

11. The method according to claim 1, wherein the flavonoid-containing compound is a flavonoid monomer.

12. The method according to claim 1, wherein the flavonoid-containing compound is a flavonoid dimer.

13. The method according to claim 1, wherein the flavonoid-containing compound is a flavonoid trimer.

14. The method according to claim 1, wherein the air occupies from 30 to 90% of the volume of the reaction vessel at 25° C. and a pressure of 101 kPa.

15. A method according to claim 1, wherein the flavonoid is selected from the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, 3′,4′-dihydroxyflavone, a dimer formed from two of the forego mg, and a trimer formed from three of the foregoing.

16. A compound selected from the group consisting of 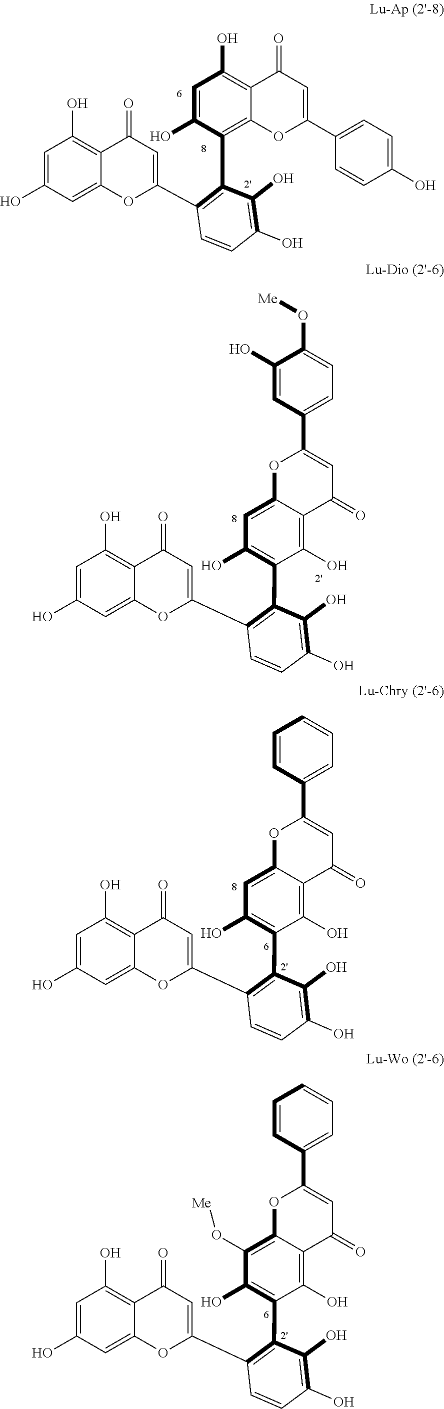 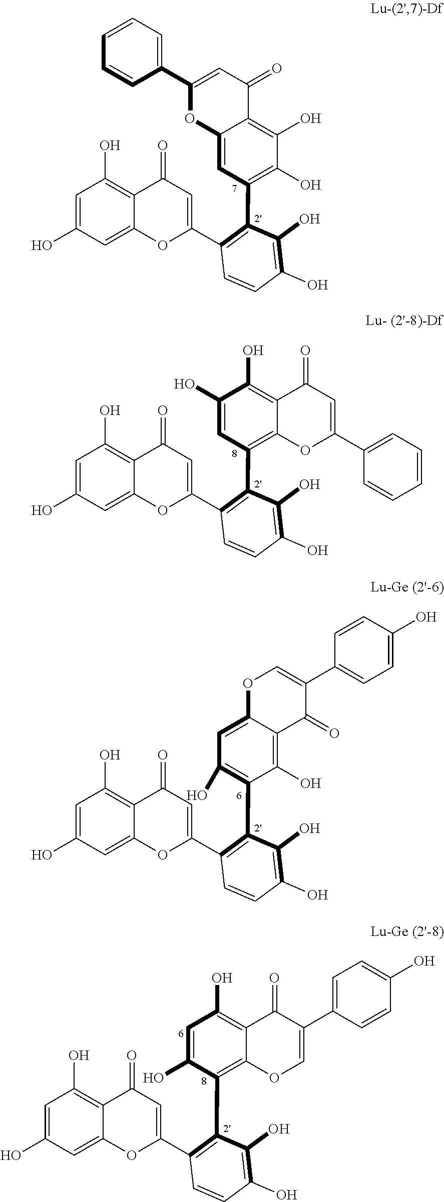 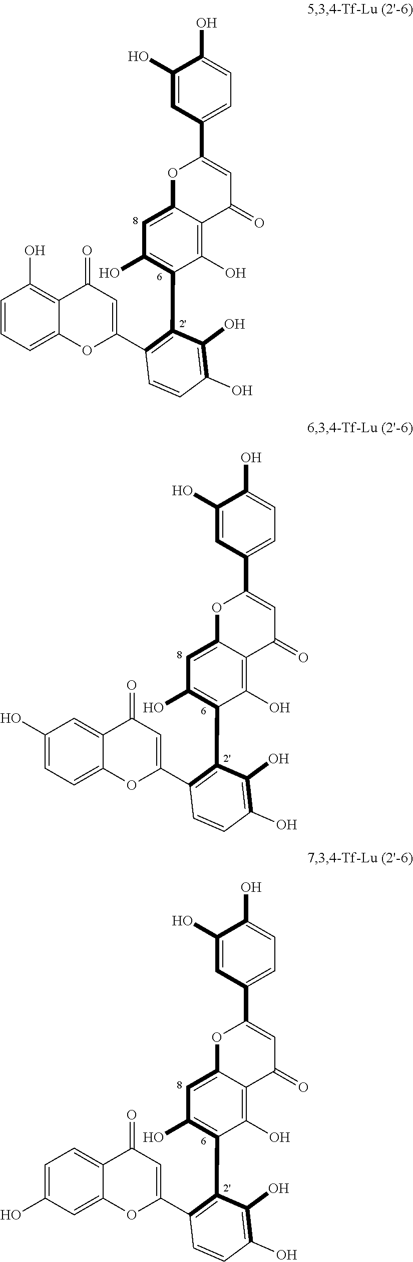 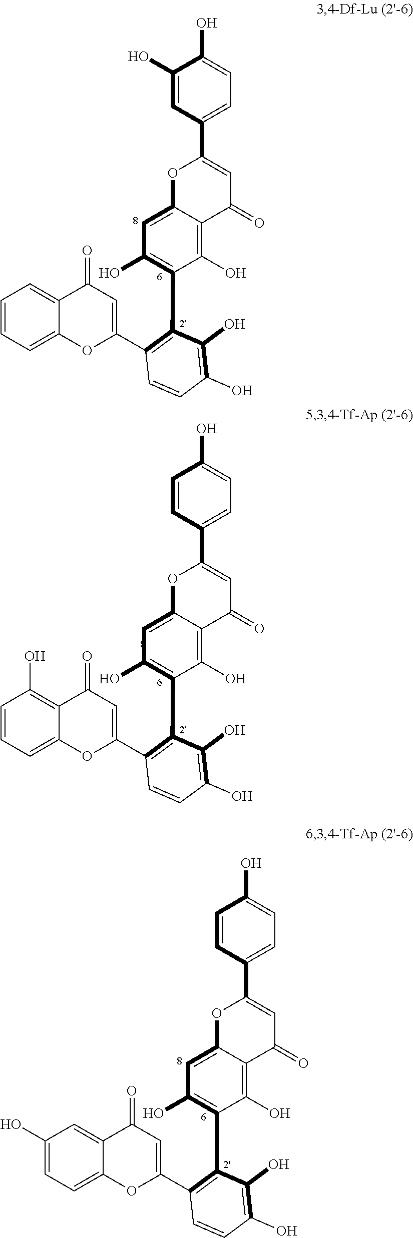 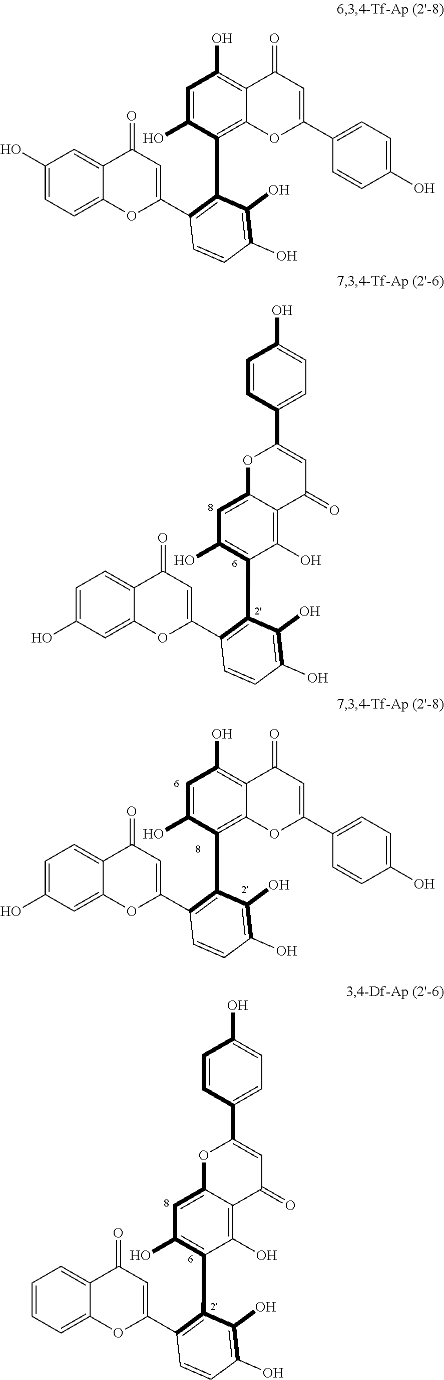 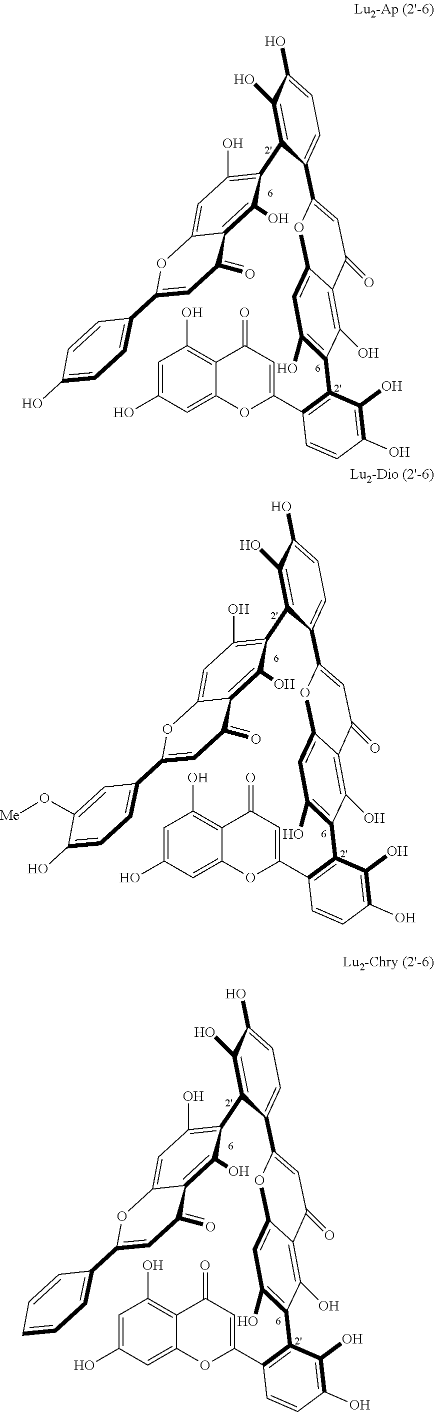 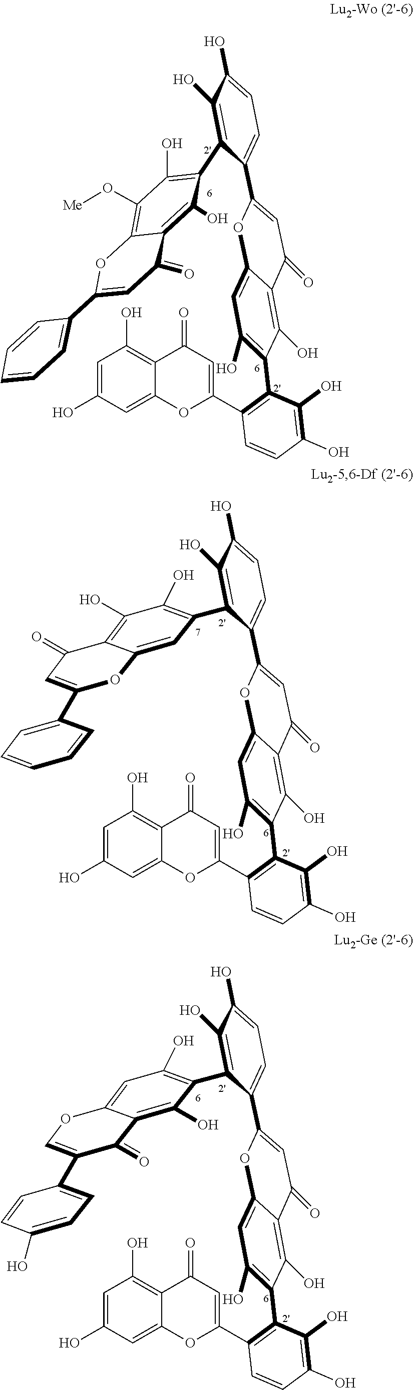 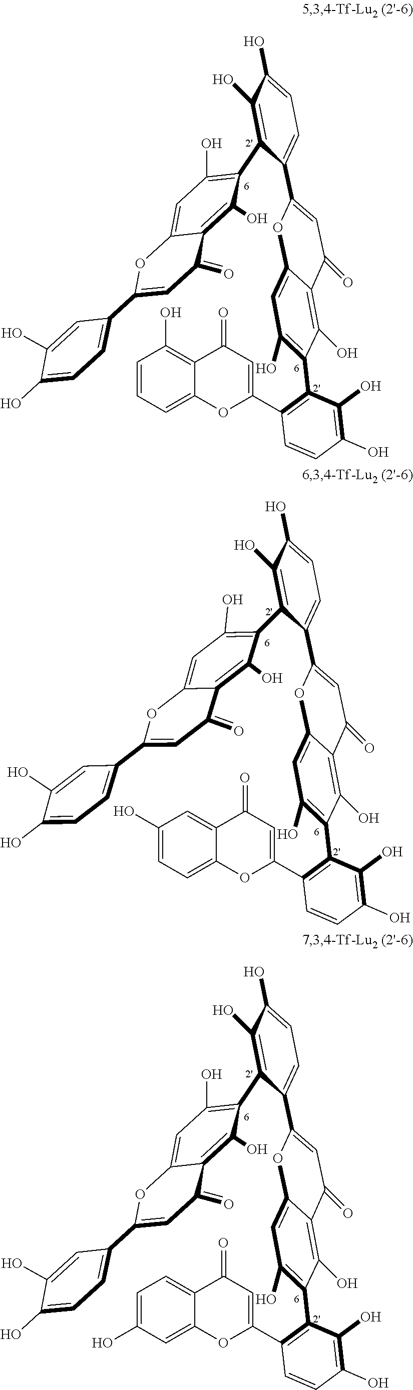 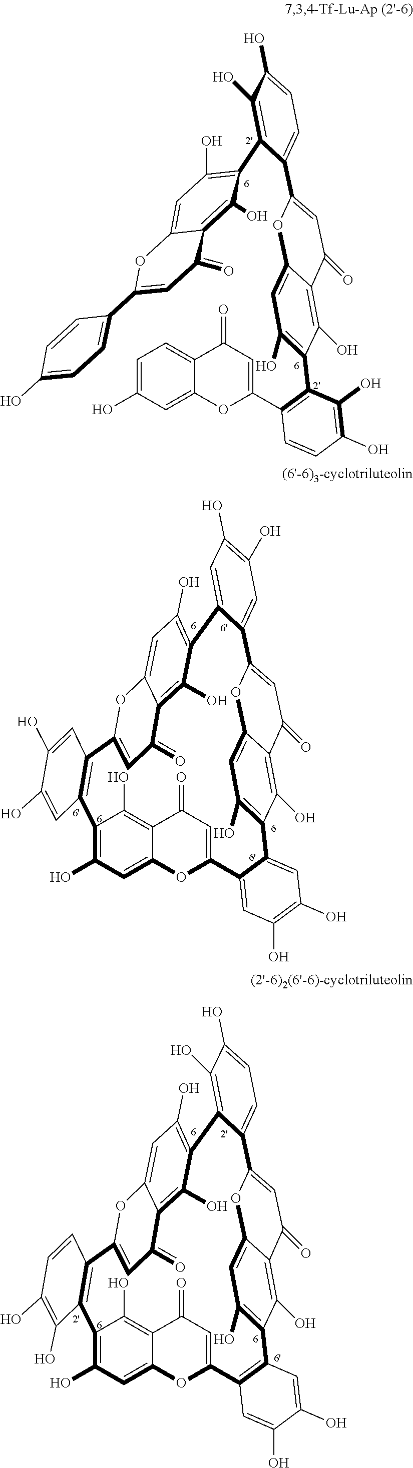
and pharmaceutically acceptable salts thereof.

17. A method of treatment of a disorder or condition selected from:
(i) a fungal infection (e.g. a candida infection); or
(ii) disorder or condition ameliorated by the inhibition of starch hydrolase (e.g. hyperglycaemia, diabetes and obesity),
which method comprises the administration of an effective amount of a compound that is:
(a) a dimer or trimer formed from one, two or three flavonoids selected from of the group consisting of luteolin, apigenin, diosmetin, chrysin, wogonin, 5,6-dihydroxyflavone, genistein, 5,3′,4′-trihydroxyflavone, 6,3′,4′-trihydroxyflavone, 7,3′,4′-trihydroxyflavone, and 3′,4′-dihydroxyflavone, or
(b) a pharmaceutically acceptable salt thereof,
to a patient in need of such treatment.

18. The method according to claim 17, wherein the compound is a compound or pharmaceutically acceptable salt according to claim 16.

19. The method according to claim 17, wherein the compound is a compound selected from the group consisting of: 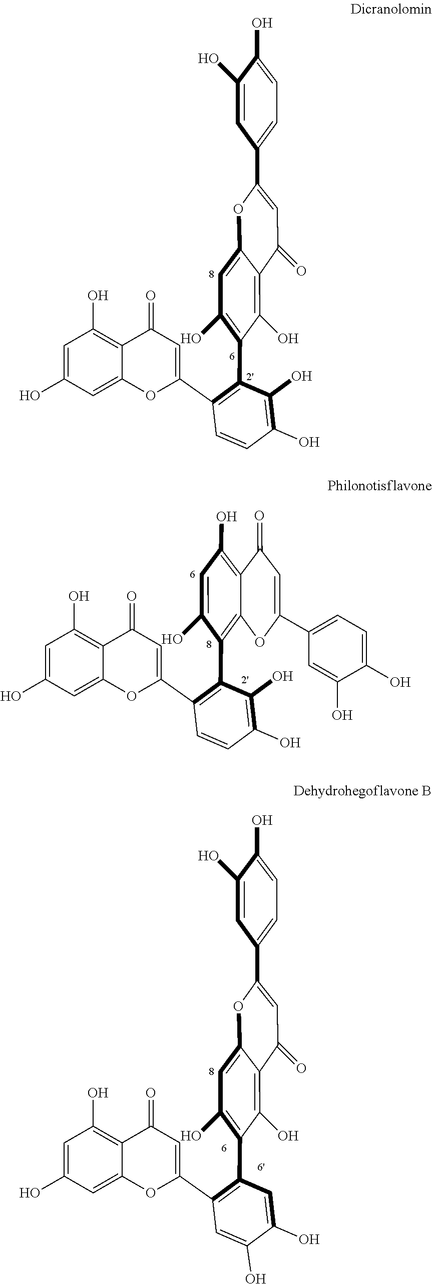 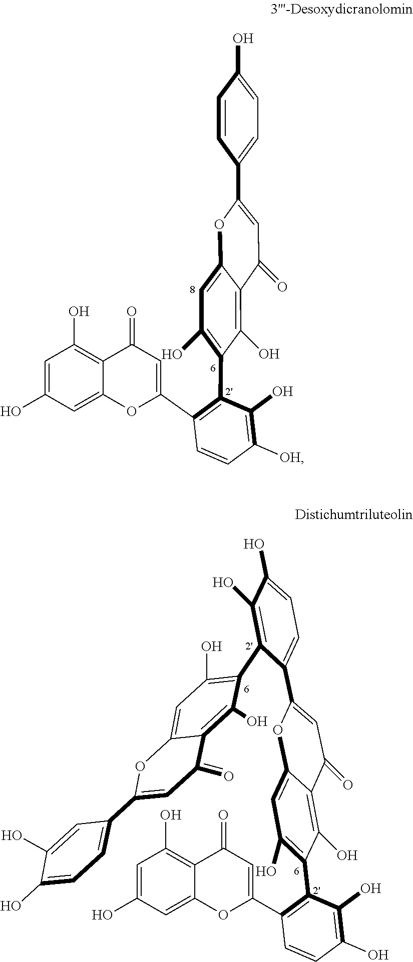
and pharmaceutically acceptable salts thereof.

20. The method according to claim 19, wherein the compound is selected from dicranolamin, 3′″-Desoxydicranolomin, distichumtriluteolin, and pharmaceutically acceptable salts thereof.
